SEQUENCE LISTING (64 sequences)

SEQ ID NO: 1 - DNA, 3888 bp, Artificial Sequence
  synthetic
atgaaaaaccaaattaacctcttcactaacaaattccaattgtcgaagacactcaggttcgagctcaagccacaggggaagaccctcgaacatattaacagtaaaggcttcataaaaaatgacgagaagagagccgactcttataaaaagatgaaggctacaatcgacgcgttccaccgcgattttatcgaccttgcaatgtcgaatgttaagcttacaaatctgatcgatttcgaggaaatatacaacgcctctaacgcagataaaaaggatgagaagtataagaccaagctgagcaagatccaagaaatcctccggaaagagatagccaaaggttttaagggcgaggaagtgaaagatatcttcagtaaaatcgataagaaagacctgatcacaaagttgttggaggaatggatcatagagaataaaattgaagatatccatttcgatccagaatttaaaaactttactacatatttcagcggctttcatcagaacagaaaaaatatgtatacagaccaagagcagtctacagcaattgcatatcggcttatacacgaaaacttgcccaggttcatcgataacatcaatatcttccagaaaatcaacaaagtgccggatttggaggaaaacctgaagaaactgtatcaggagatagaggaatacctcggaattaatgcaatcaacgaggcgttcgaactggagtatttcaacgagacgctgagtcagaaggggatcgatatatataatcttatactggggggtaggaccgctgaggaaggcaagcagaagatccaggggctcaacgaatacattaatctgtataatcagaaacaggacaaaaagaatcgggtccctaaacttaaggtgctgtataaacaaatcctctcagaccgcacccggacgtcatttcttccagatacctttgaggatgacgaggaatcgagtgcttcgcagaaagtcttggattccattaacaacttctaccttgaaaacttgatcgattacttgccgaacgacaaaaattcgaccatcaatgttctcgaaaacctgaaacttctgctggcagagctgatcaactttgaactcgacaaagtctatataaagaatgacacgtcgatcactaatatatctatgaagatattcaagaattacagtgttattagagaagccctgaattatttctatgaaaataaaatagaccctaatttcgcccataactttaataatgcaaatacggataagaaaagagagaaacttgaaaaggagaaagctaaaattactaaacaaacctacttgtccatcagtttcatagaggaggcaatccatctctatattaatgagaacagtaacgggaatcagtataagaatacatataagccgaactgcatagcgaattattttaaagatttcttcatagctgaaaataaagaaggtagcaacaaggaatttgattttattagtaagataaaggccagatacaacacgataaaaggtgtgcttaacacgccctttccggacaacaagaggcttcatcaggaaaagaataatattgataatatcaaacactttctcgactccataatggagtatcttcactttgctaagcctttggtgttgtctggatcatttgcgtttgaaaaggatgagcaattctacaccaattttgatgaactttacaatcagcttgagcttatcattccattgtataacaaagtcagaaactatgccacgcagaagccatatagtaccgagaagttcaagttgaattttgaaaactcaactttgttgaatggctgggacgtcaacaaagaggaagcaaacacttctattttgttcattaagaacgggttttattatcttgggattatggacaagaatcataataagatatttcggaacaccccgaagagtactaacaccgacatctataagaaagtcaattataaacttctccccggggctagtaaaatgcttcccaaggtgttctttggaaaaaagaacctggattattataaaccaagcaaggacatacttagaatacgcaaccacggaacgcataccaaaggcggcaagccacagagcggctttgacaaacttgacttcaaccttaatgattgtcataagctgattgacttttttaaggactcgatccagaagcacccggattggtcgaaattcaaattcaagttcagtgacacccaaatttatgaaagcatagaccagttttaccgggagcttgaaccccaggcgtatagcattacatacacgaacatagacagttcgtttatcgaggaacagatcaacgaagggaaattgtacctctttcagatttacaataaggactttagtaaattctcgaacggaaagcccaacctccacactctctattggaaggccctcttcgacgagcaaaatttgaaagacgtcacctacaagctcaacggggaggctgagattttttatcggaagaaaagcatacaacatgataggcagatcatccataaacgcaaccagccgattataaataagaaccctaacaacgaaaagaaggaatcaatattcaaatataacataattaaagacaagaggtacaccatcgataagttccagtttcatgtgccgataacgctgaatttcaaggcaaaaggcactgactatattaactacgacgtccttgattacctgaaagagaatcccgacgtgaaaataatagggctggaccggggggaacgccaccttatttatctcactcttatagatcaaaaagggaagatactcgagcaaattagcctcaatgagatagttaacaagaagcacaacatcacgacgagctaccacaatctccttgagacgaaagagattgagagggacaaagcccgcaagaattggggtacggtcgagacgattaaagaacttaaggaaggatacatctcccaagttgtgcataaaatctcgaagatgatgattgaacataatgccatcgttgtgatggaagacctgaatatgggattcaagagaggacggtttaaggtggagaaacaggtttaccaaaaactggagaagatgctcatagataagcttaattatctggttctgaaagatagacagccaaatgaacctgctggtatatacaacgctttgcaactgactaacaaattcgagtccttccaaaaactgggtaagcaaagtgggttcttgttttacgtccccgcatggaatacgtcgaaaatagatcccactacgggttttgttaacttgtttcatgtcaaatacgaatcagtgcgcaagagtcaggaattcttcaataagtttaattctataaaatacaatccaaaggaagcgatctttgagtttgactttgactacaatgaatttaccacaagagcggaaggaacaaaaacgaattggacggtgtgcacgtacggtgacagaattaagactttcagaaatcccgagaagctgaaccagtgggacaataaagagatcaatattactaccgccttcgaagactttttcggtagacacaacataacgtacggaaacgggagtgatataaaaagccaactgatatccagggaggagaaggacttcttttccgagttgattcacctttttagactcaccttgcaaatgaggaacagcaagactaactcggaaattgattatttgatatcccctgtgaaaaacgagaatgggtttttttatgatagtcgccacgccgacaaaaatcttcctaaagacgcagatgctaatggagcttatcacatcgctaaaaaaggccttcaatggataaaggaaatacagtcattcgaaggtaatgaatggaaaaaactgaagcttgataagacgaataaggggtggctcaagttcgtccaggaaaaccag

SEQ ID NO: 2 - DNA, 4389 bp, Artificial Sequence
  synthetic
atgagcgataggctggacgtcttgacgaatcagtatcctctcagtaaaactctcagattcgagctgaagcctgtcggcgccactgctgactggatcagaaagcacaacgtgatcagatatcataacggcaaactggttgggaaggacgcaattcgcttccagaactataaatacttgaagaagatgctggacgaaatgcatcggttgtttttgcaacaggcgctcgttctcgaaccgaatagtaatcaagctcaagaattgactgctcttctgagagccatagagaacaattactgtaacaataatgacctcctcgctggtgactatccttctctcagtaccgacaagactattaagatcagcaacggtctgtcaaaattgacgaccgacttgttcgataaaaagttcgaggattgggcctatcaatataaggaggacatgcccaacttttggaggcaagacatcgctgaactcgaacagaaactgcaggtttcggcgaacgcaaaggaccagaagttctacaagggaatcataaagaagcttaaaaacaagatacagaagtctgaactgaaggccgagacccacaagggactctactcgcccaccgaatcccttcaactgcttgagtggcttgttcgccgcggagatatcaaattgacatacttggagataggtaaggagaatgaaaaattgaatgaactggtccctctcgttgaactcaaggacatccacagaaacttcaataacttcgcgacttacttgtctggcttttccaagaatagggagaatgtgtattcgacaaaattcgaccggaggagtggatacaaagcgactagtgtcatagcgaggacatttgaacaaaatttgatgttttgcttgggcaatattgctaagtggcacaaagtcacagagtttataaatcaagctaataattacgagcttcttcaggagcatggaatagactggaataaacagattgcggcactcgaacacaagctcgatgtttgtttggccgaattttttgccttgaacaatttcagtcagacacttgctcagcaaggcatcgagaaatacaatcaggtcctcgctggtattgcggagatcgcaggccagcccaagactcaaggtttgaatgagttgattaaccttgcgcgccagaaactctcggctaagaggtcccagctccccacgttgcaacttctgtataagcagattctttccaaaggggacaagcccttcatcgatgactttaagtcggaccaagagctgatcgccgaattgaatgaatttgtgtccagtcagattcatggtgagcatggagcaataaaacttattaaccatgagcttgaatctttcatcaacgaggccagggcggcccaacaacagatctacgttccgaaggataagttgacagagctctccctgttgctgactggatcctggcaagcgataaaccaatggcggtacaagctttttgaccagaagcaacttgacaaacaacaaaaacaatattctttcagcctcgcacaggttgagagatggcttgcaaccgaggttgagcagcaaaatttttaccaaaccgaaaaggaacgccaacagcacaaggatacccaaccagcgaatgttacgacctcatccgatggccatagcattctgacagctttcgagcagcaagtccagactctgctcactaacatatgcgttgcagcagaaaagtataggcagctgagtgacaacctcaccgcaatcgataagcaaagggagtcggaatcttctaagggtttcgagcaaatagccgttataaaaaccttgcttgacgcgtgtaacgaacttaaccatttcctggcccgcttcacggtcaacaaaaaggacaagctgccggaggaccgcgcagagttctggtacgagaaactgcaggcctacattgacgcattcccaatatatgaactgtacaacaaggttaggaattatttgtctaagaaaccatttagtaccgagaaagttaaaatcaactttgacaattcccattttctcagcggttggaccgcggattacgagcgccatagcgctctgttgtttaaattcaatgaaaactatcttcttggtgtggtcaacgaaaacctgtcgtctgaggaagaagaaaaacttaaactggttggaggggaggaacacgctaagagattcatttacgacttccagaaaatcgataacagtaacccaccccgggtttttattcggagcaagggctcaagtttcgcgccggccgtcgagaagtatcaattgccaataggagacataattgatatctacgatcagggtaagtttaaaacagagcataagaagaagaatgaagccgagttcaaggatagtctggttcgccttatagattatttcaaacttgggttctccagacatgatagctataagcattacccgttcaaatggaaggcgtcccatcaatattccgacattgcagaattctacgcacacactgcaagcttctgttataccttgaaggaggagaacataaattttaacgtcctgcgcgagctgtcatcggcaggaaaggtttatctcttcgaaatatacaacaaagacttttccaaaaataagaggggccagggtcgggacaaccttcacacctcgtactggaaactgctcttctcagctgagaacttgaaagacgtggtgcttaagctgaatggacaggctgagatattttaccgcccggcctctctcgctgagaccaaagcttatacacacaagaaaggcgaggttcttaagcacaaagcttactccaaggtttgggaggctctcgattcacccatcggaacgagactgtcctgggacgacgcgctcaagattccgagtatcaccgaaaagacaaaccataacaatcagagagttgtccaatacaacggacaagagattggccggaaagcggaattcgccattatcaagaatagaagatattccgtggataaattcctttttcactgtccaattacactgaacttcaaggctaacggccaagataatattaacgctcgcgtgaaccagttcctggcaaataataaaaaaataaacattattggaatagatcgcggagagaagcatctgctctatatttcagtgattaaccaacagggtgaggtcctgcatcaggagtcgttcaacaccatcacaaacagttatcaaactgcaaacggagagaagcgccaagtcgttactgactatcaccagaagctggacatgtccgaggacaagcgcgataaagctcggaagtcgtggtcgactatcgaaaatattaaagaactcaaggctggctatctctctcatgtggttcatagactggctcaacttattattgagtttaatgcgattgtcgctctggaggacctgaatcatggttttaaaaggggtagattcaaaatagagaagcaagtttaccaaaaattcgagaaagccctgatcgacaagctttcgtatcttgcgttcaaggaccggaccagttgcttggagactggtcactatctgaacgcgtttcaactcacgtctaaatttaaagggttcaataaccttggaaagcaatcagggatactgttttatgttaatgctgactatacgagtacgacggatccccttactggctatataaaaaacgtgtataagacgtacagtagcgtcaaagacagtacagagttttggcaacggtttaactctataaggtatattgcaagtgaaaatagattcgaattttcatatgacttggctgacctgaaacaaaagagtttggaatccaagactaaacaaacaccacttgcgaagactcagtggacggtgagctctcatgtcaccaggagttattataatcagcagaccaaacagcatgagttgtttgaagtcacggcaagaattcaacaactcctttccaaagccgaaatctcttaccaacaccaaaatgaccttataccggcgctcgccagctgtcaatcaaaggcattgcataaagaattgatatggctgtttaacagtatcctgacgatgcgcgtgactgattccagcaaaccttcggcaacgtctgaaaatgactttattcttagccccgtggcaccctatttcgacagcaggaatctcaataagcaattgccggaaaatggagacgcgaacggcgcatataacattgcacggaaagggattatgctccttgaacgcatcggcgattttgtgccggagggaaataagaaatatcctgacctcttgatcagaaataacgactggcaaaattttgttcaacgccctgagatggttaacaagcaaaaaaagaaactggtgaaactcaaaactgaatacagtaatggctcgctgttcaacgatttggcattcaaa

SEQ ID NO: 3 - DNA, 3780 bp, Artificial Sequence
  synthetic
atgaagaagatcgactcttttactaactgctacagtctttcgaagacccttcgctttaaactcataccaataggcgctacccaatcgaacttcgaccttaacaagatgttggacgaagataaaaaaagggcagagaattattcgaaagccaagagcataatcgacaagtatcacagatttttcatagagaaggttcttagctctgtcacggagaacaaagcatttgactcattccttgaggacgtccgcgcttacgcagaactttattataggtctaacaaggacgattcggataaggcgtcaatgaagaccttggaatcgaaaatgcgcaaatttattgcgcttgccttgcagtctgatgagggattcaaagacttgtttgggcagaacctgattaagaagactttgccggaatttctggagagtgacgcagacaaagaaataattgcggaattcgatgggttctcgacgtattttactggtttcttcaacaataggaagaacatgtatagtgccgatgaccaatccacggctattagccatcgctgcatcaacgacaatttgccgaagttcctggataatgtccgcacatttaagaattcagatgtcgctaacatattgaataataacctcaagatccttaacgaagacttcgacgggatttatggcacatcagcggaggatgtcttcaatgttgattattttccgtttgtgctttcccagaagggcatagaagcctacaacagtatattgggcggatataccaactcggacggctccaaaatcaaggggctgaatgagtacatatatctctataatcagaagaacgggaacattcatcgcatacctaaaatgaagcagctctttaaacagattctctcagagcgcgagtcggtctcttttatcccagaaaaatttgattctgacgatgatgtcctttcatccataaacgattattacctggagagggacggggggaaagtgttgtcgatcgaaaaaactgttgaaaaaatagaaaaattgttttcagcagtgactgattattcaacagatgggatattcgtgaagaatgcggcggagctcacggcggtttgttcgggtgcatttggctactggggtaccgtgcaaaatgcgtggaataatgagtatgatgcccttaatggctataaagagaccgagaagtacatcgacaaaaggaagaaagcctacaagtccgtggaaagtttctctctggcggatatacaaaagtacgcagatgttagtgaaagtagtgaggccaacgcggaagtgacagaatggctgcggaacgagatcaaggaaaagtgtaatcttgcggttcaagggtatgagtccagcaaggacctgatcagcaagccgtatacggagagcaagaaactgttcaacaacgacaacgcagtcgaattgatcaaaaacgctctggatagtgtcaaggaactcgaaaatgttcttcgcttgctccttggcacgggaaaagaagaatcgaaggacgaatacttctatggagaattcctcccctgttatgagcggatatgtgaggttgatagcctgtatgataaggttagaaactacatgacccaaaaactttacaaaaccgacaaaatcaagatcaatttcagcaactcccacttcttgtcagggtgggcgcagacttattcaactaagggtgccctgatagtgaagaaagagaataattattatcttgttattgttgataagaaattgtcgaatgatgacatagtttttctgggcacaaacacacagctcagcccggcagaaaggatagtctatgattttcagaaacctgataataaaaacacacccagacttttcatcaggtccaaggggacgagctacgcaccggctgtcaaggagtacgacttgcccattagtgatattatagagatatatgacaatgagtattttaagacggaatacaggaagataaatcccaaaggctataaagaggcgcttataaagttgattgattactttaagcttggattctctaggcacgaatcctaccggtgctttaattttaaatggaaagagtcagagcaatatagtgatatatctgaattctataatgatgttgttaagtcatgttaccaactcaaatccgaatctattaatttcgactcgcttttgaaacttgttgatgaaggtaaactgtatttgttccagctttacaataaggacttctctgaacatagcaagggtaccccgaacctgcatacactgtattttaaaatgctctttgacgaaagaaacttggaaaacgtggttttcaagcttaatggcgaagcggaaatgttttatcgcgaggcgtctatctcaaaggatgatatgatagtgcatcctaaaaaccaacctataaagaataaaaacgaacagaatagccgcaagcagagtacatttgagtatgatattgtcaaagacaggcggtataccgtcgaccaatttatgcttcatatcccaatcactctgaattttacagcgaacgggggcaccaatattaacaacgaagtgagaaaggctctgaaggactgtgacaagaattatgtgataggtattgatagaggggaaaggaatcttctctacatctgtgtcgtcgacagtgaagggaggattatagaacagtactcccttaacgagattattaatgaatataacggaaatacatacagtacagactaccatgccctgctcgataaaaaggagaaggagcgcctggaatcaaggaaagcgtggaaaacggtggaaaatatcaaggagttgaaggaaggttatatcagccaagttgtgcataagatctgtgagctcgtggagaaatacgacgcagtcattgttatggaggacttgaacttcggctttaagcaaggaagaagcggtaagttcgagaaatccgtgtaccaaaaattcgagaagatgctgatagataaattgaactacttcgctgataaaaaaaagtcaccagaagagattgggtccgtcttgaacgcgtaccaactgacgaacgcgttcgaatcgtttgaaaaaatgggtaaacaaaacgggttcatcttctatgttcctgcataccttacgtcgaagattgatcctacaacggggtttgctgacctgcttcacccgtctagtaaacaatctaaagaatcgatgagggacttcgtcggacggtttgactcgattaccttcaacaagaccgagaactatttcgaattcgagcttgactataataaattcccgcgctgtaacacggactaccggaagaagtggactgtttgtacgtatggctcacgcattaagacgttccgcaacccggaaaagaactcggagtgggacaacaagacagtcgaattgactccggcttttatggcactctttgagaagtattccatcgacgtgaatggagatataaaagctcaaataatgtcagttgataaaaaagatttcttcgtggaactgattggtcttctcaggctgacactgcaaatgcggaactcggagacgggaaaagttgatagggactacctgattagcccggtgaagaatagcgagggtgtcttttataacagtgatgattacaaggggatagagaatgcttccttgcccaaagatgctgacgctaacggcgcgtacaatatcgcgcgcaagggactgtggataatagagcaaataaaagcatgtgagaatgacgcagaattgaacaagatccgccttgctattagtaatgctgaatggttggagtatgcccaaaagaag

SEQ ID NO: 4 - DNA, 4104 bp, Artificial Sequence
  synthetic
atgaagaaagagaaagagttcaaatcgtttggcgactttacgaacctttatgaaataagtaaaacccttaggtttgagttgaaacctgttgaaaacacccaaacgatgttggacgaagcagacgtttttggtaaagacaaggtcatcaaagataaatacacaaaaacaaagccgtttattgataagctccatcgggaattcgtcgacgagtcgcttaaggatgtttccctttcgggtctgaaaaagtattcagaggtgctggaaaactggaagaaaaataagaaggataaggatatcgttaaggaactgaaaaaggaggaagagcggttgagaaaagaggtcgttgagttttttgacaacacagccaagaaatgggcaaacgagaaatataaagaactgggcctgaaaaagaaagacatcggaatcctcttcgaggaatctgtgttcgacctgttgaaagaaaagtatggggaggagcaggattctttcttgaaggaggagaagggcgattttcttaagaacgaaaagggtgagaaggtgtctatctttgatgagtggaagggatttgtggggtattttaccaagttccaagagactcgcaagaacttttataagaatgatggaaccgagaccgcgttggcaactagaataatagaccaaaaccttaagagattctgcgataacattgacgactttaagaagataaagaacaaaatcgatttttcagaggtcgagaagaattttaacaaaacagcagatgtcttctcactcgacttctacaatcaatgtcttcttcagaagggaatagattcgtacaatgaatttattggcggaaaaacactggaaaatggcaagaaacttaaaggcgtgaacgagcttgtgaatgagtatagacagaaaaataagaatgaaaaagtgagcttccttaaactgctggacaaacaaatcttgtcggaaaaggaaaagctttcgtttggaatcgaaaatgacgaacagcttttggtcgtgttgaactctttttacgaaaccgctgaagaaaaaactaagatacttagaaccttgttcggggacttcgtggagcataatgagaactatgatcttgataagacgtacatatctaaagtcgccttcaacactataagtcataagtggacgaatgagacgcataagtttgaagaactgttgtatggagctatgaaagaggacaagcctataggtcttaactacgataagaaggaggactcctacaagttccctgattttatcgctctgggataccttaagaaatgtcttaataacttggattgtgacacgaagttttggaaagagaaatattatgagaataacgcggataagaaggataaggacaagggcttcctgaccgggggacagaacgcttgggaccaattcctgcagatttttattttcgaatttaaccagctcttcaattcagaggcctttgataataaagggaaagaaatcaaaataggctatgacaactttagaaaggattttgaggaaatcatcaaccagaaagattttaagaacgacgaaaacctgaagatcgctatcaagaactttgctgactcggttttgtggatctatcagatggccaaatacttcgcgattgaaaagaaaaggggttgggacgacgactttgagctcagcgagttctatacaaacccatcgaacggctatagcctgttctatgaccgcgcgtacgaggaaatagttcaaaaatacaatgatttgcgcaattacctcaccaagaagccgtataaggaagataaatggaaacttaacttcgaaaacccaaccttggctaacggattcgataagaacaaggaatcagataactcgacagtgatcttgagaaagaaacgcaagtactatcttggcctcatgaagaagggaaataacaaaattttcgaagatcggaataaggctgagttcataagaaatatcgagtcgggagcctacgaaaaaatggcatataaatatctccccgacgtggcaaagatgatccccaaatgttccacacagctcaacgaggcaaagaatcactttcgcaactcggcggacgacttggagatcaaaaagtccttttctaaccccctgaagataactaaacgcatctttgatctcaacaacatacaatacgacaagacaaatgtgtcaaagaagataagcggggataataaggggattaaaatctttcagaaagaatactacaaaatctccggagacttcgatgtttacaaaagtgctctcaatgactggatagacttctgtaaggacttcctgtcaaagtatgacagcacaaaagacttcgacttcagtattttgcgcaaaacgaaagactacaagtcgctggacgaattctatgtggatgtcgccaagataacgtataaaattagcttcactcccgtctctgaatcttacatagatcagaagaataaaaacggtgagttgtatcttttcgaaatatacaatcaagactttgcaaaaggcaagatgggggccaaaaaccttcacacgctttatttcgaaaacgtcttttctccggagaacatatcgaaaaactttcccatcaaattgaatggaaacgccgaattgttttttaggccaaagtcaatagagagtaagaaggagaagcgcaattttgtcagagagatcgttaataagaaacggtactcggaggataaaattttctttcactgcccgatcacgcttaatcgcgagactggatccatttatagatttaacaactacgttaataacttcctgtcggaaaacaatattaatataatcggcgtcgaccggggtgaaaaacacctggcctattattcagtcatcgacaaaaatggagttaaaataggcggtggctcgttcaatgagatcaataaggttgattatgcaaaaaagttggaggagcgcgccggggaacgggaacagtcacgcaaagattggcaggtggtcgaagggattaaggacttgaagaaggggtacatatcccaagtggtcagagaactggcggatcttgctatcaagcacaacgcgatcatcgttctggaagaccttaacatgaggtttaaacaaataagaggcggaatagagaaatctatttaccagcaacttgagaaagcattgatcgacaagcttagtttccttgtcgaaaaaggcgaaaaggaccccaatcaagcaggtcatatactgaaagcttatcaactcgcagcaccgttcacgtcttttaaggatatgggtaaacaaacaggcatcgttttctatacacaagcctcctatacatccaagacctgtccaaattgtgggttcagaaaaaataataacaagttttacttcgagaataatatcggcaaagcgcaagacgcgctcaagaaattgaagacttttgagtacgacagcgaaaacaaatgctttgggctctcctactgcttgtcagattttgcaaacaaagaggaagttgagaaaaacaagaataagaaacggaataatgcgccatacagtgacattgagaaaaaggactgctttgaactgtctacgaaagacgcggtcagatatcggtggcatgataagaacacggaaagagggaagacgttttttgaaggtgagtctgtttatgaagagaaagaggaaaaagaaatcgggcaaacaaagagagggcttgtcaaggaatacgacatcagtaagtgtttgatcggattgttcgaaaaaactggattggactacaaacaaaatctccttgataagatcaactcgggtaagtttgacggtactttttataaaaatctctttaattatctgaacttgttgttcgagataagaaactctatatctggtacagaaattgattacatttcctgtccggaatgccaatttcacactgacaagtccaaaactatcaaaaacggagacgacaatggcagttacaacatcgcgcgcaaagggatgataatcctggataagattaagcaatttaagaaggaaaatggatctttggataagatgggctggggagagctttttattgatctggaagagtgggacaagtttgctcaaaagaagaacaacaacataattgataaa

SEQ ID NO: 5 - DNA, 3714 bp, Artificial Sequence
  synthetic
atgaaggatttttatcagttcacgaacctctacgcactgtccaaaacgttgcgcttttcacttataccgacccccgcgacgaaacagatgctcgaagatgcaaaggtctttgagaaggacgagacgatacagaaaaaatatgaagccacaaagccgtacttcgataggctccacagagagtttgcgctcgaggcacttcaggaccaaaagttggactttaagaattatctcgaactgtacaggaaatataaggctgacaaaaaagccagtgggaaacttttgataaatattgaaaaggacctcaggaaagaagtcgttaagcttttcgataagcagggtgagaaatgggctaaacaatatccgggactcaaaaataagaatatcggtgttctgttcaaagaggccgtttttactgttatattgaaagagcggtatgggaatgagaaagaaacacaaatcctggacgaaagcagtggccagcttgtgagtatattcgattcttggaagggttttattggctattttaagaagtttcacgagacgagaaagaatttctataaggacgacggaacgagtactgcgctggcaaccaggatcatagatcaaaatttgaagcggttctgcgacaacatccttatatttgaaagtacgaaggagaaggtggacttttcagaggttgaaatatctttcggcaagccactgtcagaagtgttcactcttgagttctataatacgtgcttcctccaaaacggcatagacttttatacgaagatccttggcggtgaaaccctgcagaatggcgagaaggttaaagggctcaatgaatgcattaatctccacaaacaaaaaactggtgagaagcttcccttttttaaatcgttggataaacaaattttgtcagagaaagacaagttttttatagacgaaatcagcaacgagacgcagctcttggaggtcctcaaatcttttgtggcatccgcggagtcgaagacagacactattaaaacgcttgtcgacgacttcgtcaaagaccaagacaagtacgaccttaattatatatatttttcgaacgatggccttaacacaataacgagaaagtggactacggaaacgcaggttttcgaagaggctctctatacggctctcaaggccgcgaaggtggtttcgagcagcgctaagaaaaatgaaggtgggtattcttttcctgattttatacctttcgctcacctgaagacagcgcttgaaagcatcaaaattgacggcacgatatggagggataattttaatgcaattgagaatttcgaagaaaaaagcatctgggcgcaatttctggcaatatacaattttgagcttagcaatctcttcgaaacggagatcaaaaatccggaaatcggtaattgtccgacgataggatataacgtttacaaacaagatttcgaagaactcttgaagagcttcgtctatgatcccaacgcgaaggtgactattaaaaattttgccgacaacgtgctctcaatttatcagatggctaagtactttgcagtcgaaaagaagagaggttggaacaccgattatgagttggacgttttttatacagatccacagaacggttatttgcaatactacgagaacgcatacgaagagatagtccaggtgtataataaattgcgcaattacctgacgaaaaagccatattcggaggagaagtggaaactcaatttcgattctggtactcctattaagtatacgacaagggcaataatttttaataacacaacaaacgagcggtattaccttggcttgcttaaaaaaggtgtggctaagccccgcgaattcgaaccgataaataataacatcatatcctcgggggaatttaggagaatgatcatacaacaactgaaatttcaaactttggcggggaaggggtacgttcgggacttcggggtcaagtattcagaggataaagacggcgtgaaacatctccagcagctcattaagaaacagtacctctccaaatatccttgtttgaagaaaatcgctgatggggtttacaacgacaaaaaagctttcgatgctgacataaaggacgtgctgctggagacgtacaatctggattttcaaccaataagcgaagaatttatccttaataagaatcgcctgggagaaatttacctcttcgaaatacataataaagactggaatttgaaagacggaaagaacaagtctggttcaaaaaacttgcataccatgtatttcgagtccttgtttgtcgataagacgaccttcaaactcaataatgagggtgcggaagtcttttatagaccggccactaacgagggtaagctcggaacaaaaaaggatcgcaacgggaagattattattaaccataaacggtatgccaccgataagatcttgttccattgccccattggtctgaataaggacgcaggaaagtcttacacgttcaatgccaaaataaataacatgctggctaacaacccggatattaatattattggggtcgacaggggagagaaacatctggcgtactacagcgtgattacgcagaaggggaaaatcctggaccggggatccctgaacaaagtcgaaggcggggacaagcaagagattgactatgcaaaaaaacttgaggaaacagccaaaaacagagagcaggcaaggaaggactggcaagcggttgaagggataaaagacctcaagcgcggatacatttcgcaagtcgtgcggaaactggctgatctggcgatcgaacacaatgcgataatagtcttcgaagatcttaacatgcggtttaagcaaatcagagggggtatcgaaaagagcgtgtaccaacagctcgaaaaagccctcatcgataagctttctttcttggttatgaaaggggaggctgatcccgaaaaagctggacacctcctgaaggcgtaccaacttgttgcccctttcgagtccttccagtccatggggaaacaaacaggtataatattctatacacaggctaactacacaagtaaaatagacccaattacgggttggagacccaatttgtacctcaagtatacttcagcggaaaaagcaaaggctgatatcctcaagttcagcaaaatcgaattcgtgaataaccggtttgaattgacttacgacataaaaaattttgtcttggataaaaaggtcgtgctctcgaataaaacaaagtggacggtttgctcaagcgtggagcggttccgctggaatagacgccttgagtcaaatcagggaaattacgagcattacgagaatctcactgaaaatctgtcttctctctttaaagactttggatttgaaatagaacagaacataatcaggcaagttgaacagttggctacgaagggaaacgagcagttctttaggagcttcatcttctatgtgaacctcatctttcagatccgcaatacggatgcgaaagctaaggatcagaacaaagaagatttcatcctctcaccagtcgagcctttctttgactctcgcacaccggaaaagttcggagaaaaccttcccgagaatggggacgacaacggcgcatttaatatcgcacgcaaaggtatcattatgcttaacaagataagtgcatacaagcaggaagtgggtaacgtggacaagataatatggaaggatctgttcatatctgcagcagaatgggataatttcacccaagaa

SEQ ID NO: 6 - DNA, 3777 bp, Artificial Sequence
  synthetic
atggaatcaaacaataaaatctttaccgaaaccatcggcacttccagcatagcgaaaacgatgcgcaattcactcgttcctaccgagagcacgaagcgcaacattgaaaaaaatgggattataatagacgaccaactgagggccgagaaacggcaacagctcaaagaaatcatggacgaatactatcggacttacatagacaataagctgtcgaacgttgccctcacgagaacaatagattggaaggaactttttcaagcgatcgaagataattacaagcaaaacacgaccaagaccaaaaacgaattggaaaaaaagcaaaaggagaagcggacggagatttacaaaatcctctctgacgacgaaaagttcaaacagttgttcaatgcgaagcttctcactaacgttcttccggaattcataaagaatcagaacatagacaatgaggaaaaacaggaaaaaatttctacagtcgaactttttcaaaggtttacgagttctttcaccgatttctttaagaaccggaagaacgttttctccaaagacgaaatctcgacatcgatttgctatagggttgtccaggaaaacgcatggatattctatcagaatcttctggcttttgaagagatcaagaaaacggctgaacaggaaatcgagaagatcgaagcagagaaccgcgacagcatatctgactatagtttgaaggaaattttcgatttcgatttctacggccttctgctgaatcagggcgggatacgcttctacaatgacgtctgcggcaagattaattatcatatgaatttgtacggtcaaaaacacaatataaagtcaaataaatttaaaatgaagcggatgcataaacaaatattgtcgatagatgaatcgaccttcgaagtgccgacgatgtttgagaacgataaagaggtgtaccaggtcctcaatgagtttctgtctgatctggcttccaagaagatcttggaaagggtggaaaaaataggggagaacgtgagtgaatacgagatcaataagatttatattcaaagcaaaaattttgaaaaattctcttcatttatgtgcgggaactggcaaattatcaacgattcactgaagacttattacaatgagaagataaagtcgaaaggtaaagcaaaggaggagaaagtcaaaaaagctataaaggcaatcgaatataagtctcttgctgacataaaccagcttgtcgagagatacaataacgacgaactgaatcgcaaagcagaagagtatatatcggcgataaatgagaaaatcaaggatctcgacgtgaatgaaattgagtatgatgaaaaaattaatcttattgaaaatgagactaaatccgaggagatcaaaagcaagctcgacagcataatggaaatcatgcactggacgaaaatgtttatcattgaagaggagatagaaaaggatgttaacttctataatgaaatagaggagatttacgacgaactgcaaccccttgtcacgatctacaatcggatcaggaattacgtcactcagaagccttattccgaagaaaagattaaactgaacttcggaattccaacacttgccaacggctggagcaaaaccaaagaatacgacaacaatgcgattatcatgatccgggacggaaaatactacctcggcatcttcaatgccaagaataaaccggacaaaaaaattatggaaggacaccaaagcgaggagaatggcgattacaaaaagatgatctaccgcttgcttcctggccctaacaagatgctgccaaaggttttcatgtctaagactggcatagcggagtataagccgtctcagtacattctcgaatgttatgagcaaaataagcacataaaatctgacaaaaattttgatataaaattttgtagagaccttatcgattttttcaagacatcgattaatcggcatcctgaatggtcgaagttcaactttaagttctctgagacatccgaatacgaagatatctcaacattctaccgggaggttgaaaaacaaggttacaaaatagagtggacctacatcagcgaaaaagaaatcaaagaacttgacgaaaatgggcagttgtatttgttccagatttataataaggatttttctgagaaaagtaagggcaaggagaatctgcacacgatgtatttgaaaaatctgttctcggaagagaatcttaaaaatatcgtccttaaacttaatggtgaagcggaggtgtttttcaggaaatcttcaataaaaaagcccattattcataaaaagggttccgttttggtcaacaagacgtataacgaaaacggtgaacggaagtccataccagaggagcagtatacagaaatatataaatatcttaacagtattggaactaatgagctgtcagaaaaatcgaagaaacttatggaagaaggaaaagtcgaatactataaagcaaactacgacattgtgaaagattaccgctattcagtcgataaattctttattcatctgccaatgacaataaattttaaggcagccgggttttcacctattaataatattgcgttgaaaaacatcgccctgaaagatgatatgcatattataggcatagatcggggcgagcggaatcttatctatgtctccgtcattgatactaaaggcaatatcgtcgaacagaggaattttaacatagtcaatggcattgattacaaagagaagttgaaacaaaaagagttggatcgggacaatgcgagaaaaaactggaaggagattggcaagatcaaggaccttaaagagggctacttgtctctcgtcgttcatgagattgctaaactggttgtcaaatataacgcaataattactatggaagacttgaatcaaggttttaaaaggggcagatttaaggttgaacggcaagtgtaccagaagttcgaaacaatgttgataaataaattgaattatctggtggacaaggacctcgccgttgaccaggagggagggctcctgcggggctatcagttgacgtatatcccagagtctctcaaagtgctcggacggcagtgcgggtacatattctacgtccccgccgcatacacttctaaaatcgatccgacaacaggattcgtggctatatttaattataaaggaatgacagacaaagacttcgtcacatcgtttgactcaataaaatatgacgacgaacggggcctctttgctttcgaattcgactatgagaatttcgtcacgcacaaggtcgaaatggcacggaataagtggacggtgtacacatacggagaaaggataaagagaaagttcaaaaatggcagctgggataccgcagagaaagttgacctgacataccagatgagatctatactggagaagtatgaaatagagtacaacaaggggcaagacatcttggaacaaatagaagaactcgatgagaaggcgcaaaacggcatatgcaaggagattaaatatctggtcaaggacatcgtccaaatgcgcaactccctcccagacaacgctgccgaggattatgatgcaatcattagtcctgtgattaacaacaatggggagttcttcgactcaacaaggggggacgaggacaaaccgcttgatgcagacgcgaatggcgcatattgtattgctttgaagggactgtacgaggttatgcaaataaaaaaaaattggaatgaagagacggaatttccgcgcaaggaactcaagatacggcatcaggattggttcgattttatccaaaacaaaagatacctg

SEQ ID NO: 7 - DNA, 3894 bp, Artificial Sequence
  synthetic
atgaccaagacattcgactctgaatttttcaacctttacagtcttcagaaaaccgtcagatttgagctcaaaccggtcggtgaaacggcctcattcgtcgaggactttaagaacgaaggcctgaagcgggttgttagcgaagacgagaggcgcgcagtggattatcaaaaagtgaaggaaatcattgacgattatcatcgcgatttcattgaagagtcactgaactacttcccagaacaggtttcaaaagacgccttggaacaggcttttcacttgtaccaaaaattgaaggccgccaaggtggaagaacgggagaaagccttgaaggagtgggaagcgctccaaaaaaaattgagggagaaagtggtgaaatgctttagtgattcaaacaaggccaggtttagtagaatagataaaaaggaactcataaaggaggatcttattaattggctcgtggctcaaaaccgcgaagacgacattcccacagtggaaaccttcaacaacttcaccacttatttcaccgggtttcacgaaaatagaaagaatatatacagcaaggatgaccatgcaaccgctatatctttccgcttgatacacgaaaacctcccgaaattcttcgacaatgtcatctcgttcaacaagctcaaggaaggttttcccgaattgaaattcgataaggtgaaagaagacttggaggtggactatgacctgaaacatgcctttgaaatagaatattttgtcaatttcgtgacacaagcggggatcgatcaatataattacttgctgggcggcaagacgttggaggatggcactaaaaagcagggcatgaatgagcagatcaatcttttcaagcaacagcaaactagagacaaggctagacagatcccaaaattgatcccattgttcaaacagatcctttcggagaggaccgaatctcagtcgtttatccccaaacagttcgaaagtgaccaggaactgtttgattcccttcaaaaattgcataacaactgtcaggacaagttcactgtcttgcagcaagccatcttgggcctggcggaagcagatttgaaaaaagtgtttataaagacctcggacttgaatgcgcttagcaatacgatttttggcaattactctgtgtttagcgatgcactcaacctttataaagagagcctgaagaccaagaaagcccaagaagcttttgagaaacttcctgcgcattcgatccatgatcttattcaatatcttgagcagtttaactcaagtcttgacgcggaaaagcagcagtccactgacacggttctgaactatttcatcaaaacagatgaactctactcgagatttattaaatcaacctcggaggcatttactcaggtgcaaccgcttttcgagctcgaagccctttccagcaaacgcagaccaccggaatccgaagatgaaggtgctaaaggacaagaagggttcgaacaaattaagcggattaaggcatatcttgatacgcttatggaagcggtccacttcgcgaagccgctttacctggtcaaggggcggaaaatgatcgagggtcttgacaaagatcagtctttttacgaggcgtttgagatggcttaccaggagctcgaaagtttgataatcccaatatacaacaaggccagatcctatctctcgcggaagccttttaaggcagataagttcaagattaactttgacaacaacacgcttttgtcaggctgggacgccaataaggaaactgccaacgcgagtatcctctttaaaaaagacggtctttactatcttggtataatgccaaaaggtaaaacatttttgtttgattatttcgtcagctcggaagattccgaaaaacttaaacagcggaggcaaaagacggccgaggaggccctcgcgcaggatggtgagagctattttgaaaaaattcgctataagttgctccccggagctagtaaaatgttgcccaaggtcttctttagtaataagaacattggcttctacaatccctcagatgatatccttaggataaggaatacagcgagccacacgaaaaacggcacaccgcaaaaaggacattccaaagtggagttcaatctgaacgattgccataagatgatcgatttctttaaatcctctatccaaaaacaccccgaatggggctcgttcggtttcactttttcggacacgagtgattttgaagacatgagtgcgttctatagggaggttgaaaatcaagggtatgtcatctcgtttgataagattaaagaaacgtatattcaaagccaggttgaacagggcaatctctatcttttccagatatacaataaagacttttcgccatactctaagggaaagccgaatctccatactctgtattggaaagccctcttcgaggaagcgaaccttaacaatgttgtggcaaagcttaatggggaagcagaaatatttttccgccgccactcaataaaggccagcgacaaagttgtgcatcctgcgaaccaggccattgataataagaacccgcacacagaaaagactcagagtaccttcgaatatgatcttgttaaagacaagcgctacacacaggacaagttctttttccacgttccaatatcactcaatttcaaggcgcaaggagtcagcaaatttaatgataaagtcaatggattcctcaaagggaatccggatgtgaacattattgggatagacagaggcgagagacacctgctgtactttacggttgtgaatcaaaagggggaaattttggtccaggagtctctcaacacgctcatgagtgacaagggccacgtcaatgattaccaacagaagctggacaaaaaagaacaggagagggatgcagcaagaaaaagctggacgacggtggagaatatcaaggagctgaaagaaggttacttgtcgcacgttgtgcataagctggctcatctgatcattaaatacaatgccatcgtctgcttggaggacctcaactttggttttaaacgcgggagatttaaggtcgagaaacaggtttaccaaaagttcgaaaaagcgctcatcgataagctgaactacctggtgtttaaagaaaaagaacttggggaagtcgggcactatctcacagcgtaccagcttacagcgccttttgagagcttcaaaaaactcggcaaacagtcgggcatccttttctacgtcccggcggattatacgtcaaagatagaccccacaacagggtttgttaacttcttggatcttcggtatcaaagcgtcgaaaaggctaagcaacttctctccgacttcaacgctataaggtttaatagcgtccaaaattatttcgagtttgaaatagattataagaagctcacaccaaagcggaaggttgggacccagtctaagtgggtcatatgcacatacggcgatgtgaggtaccagaataggcgcaatcaaaagggtcactgggagaccgaagaggttaatgtgactgagaagcttaaggctctttttgcctcagactctaaaacaactactgtgatcgattacgccaacgatgacaacctcattgacgtgatcctcgaacaagataaggcctccttttttaaagaactcctttggcttctgaagctgacgatgacactccgccactccaaaatcaagagcgaggacgactttatactgtcacctgtgaagaatgaacaaggcgaattttacgactcgaggaaagcaggcgaagtttggcccaaagacgctgacgccaatggggcatatcacatcgctcttaagggactctggaacttgcaacagattaatcagtgggagaaagggaagaccttgaatctcgcgataaagaatcaagattggttcagctttattcaagaaaagccgtaccaagaa

SEQ ID NO: 8 - DNA, 3969 bp, Artificial Sequence
  synthetic
atgacgaacactaagcagaactcggtttttcaagactttactcgcaagtatgccctctcgaagactttgaggtttgagttgaggcccgttggaaacactcgggaaatgcttgatgccgcgggcgttttcgaaaaggatcgcataatcaaggagaaatacgagcgcaccaaaccttacttcgaccggttgcatagagaattcgtgcaggagtcattgagcgatgtcgctttgactggcgtcgccgaatacctttcaatcttttcagagtggcgcactgacaaaaaggataaaaccaaagctaagcagcttagtgatattgagaaatcgcttcgcaatgaggtcaccaggttcttcgaggaaactgcaaaaatatgggctgagaagtatagttcgataaagattaagaaaagcgggatagactttctctttgaagaggggatttttcgggtcctcaaagagcgctatggaaacgaagatgacgcctttgtggaagatgatgcaggtaacaggatcagtattttcgatcaatggaagggatttgtggggtattttactaagttttatgaaacgaggaagaacttttataaggacgacggaaaagcgtcaaggatcgcgacacgcatagtcgacgaaaacctcaagagattttgtgacaatataatagccttcgatagattgaagcaagtgttggacctttcagaggtgcaaggtcatcttaaggactcgctgaccgaagtgttctccccccgcttctacgacgaatgtctgctccggcagggaatagaccggtataataccatattgggcggggatagcatggaccaaaacgtcaagggcattaaccagtatgtgaataagtaccttcaggatcacaagggggagcggcttccgtttctcaaacctctggacaagcagatactgagtgaaaaagaatcgtttcttgaggggattgaaagtgatggtgaacttctgagtgtcttggaattgttccagaaaaacgcgggcaagaaggtcggtatatttaggtcgttgatggaaaaatttgttagcaagaacgatgcttttgatctttcgaaaatatacttgtccaaagaaggctttaacacgataagccgcaaatggacggataacactagggacttggagtcatcactccataaagctatgaaagcggacaaaccgacgggtctgtcctacgataagacggaggatttgtataagtttcccgaccacatagcgctgagttacctgcaggaagcaatcagtgcgcagaaccaagagaatacggtttggaaggaacggtactacaagacggaagagaacagctcgggagtgcttttcgggaacgagtcagtttgggagcaattcctccatattctgtcatttgaaatcagtagcctgtatagcacggagacgactcacacgatagccgaaaaagaagattttgctaccgccctgataaacgctgaacgcttcgaggaggtcttttcttcagatagtatcgtgcgcacgattcaaactaaggtcggctacgacgtttttgaacaaaaaatgacttctgtcagtggcccagaatttggacctgatgagaaggtcttcataaaaaactatgccgatagcgtccttcggatttatcaaatggctaaatatttttcacttgagaagaagggcgtttggaatgaggagtacgagttggactctttctattctgagcccgagcatggttatctggtttttcatgaaaatgtgcgcgaagaaatagttaaagtctacgatgccctcagaaactacctgacgaagaagccgtattccgaagaaaaatggaagctgaatttcgagaaccccacactggccgatggctgggataagaacaaggagtcggataattcggctgttatactgcggaaagacggtcagtattacttgggggtgatgaaaaaaggcaagacgcatatcttttcggaacggaatgaaaaagaaatgggtattggaggtggcgggatgtcttacgagaagatggtttataaactcttccctgatccagctaaaatgatgccaaaggtgtgtttctcggctaaaggattggagttctttaaccctccagcagatataatggaaatctatgaaaaagggacctttaagaagggggagaactttgatataggcagcatgcggagactgattgatttctataaggagtgcctcgcgctctatcccgggtggaaagggtatgatttttcaaatgtgaaagagtccgccacatatgtgacgaacatcggggagttttattcagacgttgcaaaagccggttacaaggtcgagttcgtggctatttcagagtcctatgtgggggagaagaacgaagcgggtgaactctacctgttcgagatacataacaaagattggaacctgaaggattcgaaaagaaaagatggcgctaagaacctccatacgctgtattttgaatctctgttcagcgaggagaacgtggaagctgactttccaatgaagcttaatgggcaagctgaacttttctataggccggcaactgctaccgataaactcggattcaagaagaacgcagatgggactgacatggttggtaaaaacggtcagaaagtggtgaaccacaaacgctactcggaagacaagatactgttccacgtccctatgacaatgaatcggggcgccggcaatatttataggttcaatgacgagattaatgaattgcttgcaggtaacccagccataaatatcataggagtcgacaggggggagaaacatctggcctactattccgtcattaaccagaagggcgaggttcttgaaagtggtagtcttaacacagtcaacgggatcgattatgcgggcaaactggatgaaagagccaagggaagagaacaggctcgcaaagattggcaagcagttgaagggattaaggatctgaagaaaggttatgttagccaagtggtgaggaaactggcggatcttgctatcgaatacaacgctatcatagttcttgaggatttgaatatgaggtttaagcagatccgcggaggtatcgagaaatcagcataccaacagctggaaaaggcactgattgacaaacttagctttctcgtggaaaagggtgagaaagatagtgagtccgctggacacctgttgagagcttaccagttggccgccccctttgaaagtttccaagaaattggtaagcagacgggaattatattttacacccaggcatcctacacatcgaaaatagatccggccacgggatggagaccgaatctttacttcaagtatacctcggccaaaaaggcgaaagaggacatagaaaggtttgacgccattcattttagagacggctatttcgagttctcctatgacttgggcaagatcgttaactcggagaagggagcgacaatcttcccggagcgcacagactggactgtgggctcttgcgtggaaaggtacagatggaacagaaagctgaatgcggggaaaggggggtatgactattactctgacttgacggaggactttcggtcgctttttaccagtgttggaattggggaagagggggacattaaagcacagattgctgcactcgactccgaaggcaacgaaagattttttagagatttcattttctttttttcgcttatatgtcaaatcagaaacacggaccccgacgcaaaagatcctgacaaagcggattttatcttgtcgcccgtcgcgcccttttttgatagcaggaccccagaggcattcggcacaaagttgccaaaaaacggtgacgacaacggcgcgtacaatatcgcccggaaaggtatcattattttggataaaatttcagattataagcgggctaacgggtcgtgcaaaaatctcggatggggaggcctttacatatcacacgccgagtgggacgatttcgcaagaaagaacgctctgaagaacagcggc

SEQ ID NO: 9 - DNA, 3780 bp, Artificial Sequence
  synthetic
atggattcactgaaagacttcacgaacctgtatccggtgagtaagacacttcgctttgagttgaaaccagtgggcaagactctcgaaaatatagaaaaagctgggatcctcaaggaggatgaacatcgcgcagaatcgtatagaagggtcaagaagataatagatacttaccataaggtgttcatagattcatcacttgagaacatggccaagatgggtatagaaaacgaaataaaggcgatgctccaatccttttgtgagttgtataagaaagatcaccgcacagaaggagaggacaaggcgctggacaagattcgggccgttctccgggggttgatcgtcggagcgttcactggtgtttgcggacggcgcgagaacaccgtccaaaatgaaaagtacgagtccctctttaaggaaaaactgattaaagagatcttgcctgattttgttctgtcaacggaggctgaatcgctccccttttctgtcgaggaggctaccaggagtctgaaggagttcgactccttcacctcctacttcgcgggtttttacgaaaacaggaaaaacatttactctactaagcctcagagtaccgccattgcctatcggctcatccatgagaacctgcccaaatttattgacaacatattggtctttcagaagattaaggagccgatcgcgaaagaacttgaacatatcagagcagatttctctgcaggtggctacatcaagaaagatgagagattggaagatattttctccctcaattattacatccacgtcctgtctcaagcgggaattgagaagtataatgctcttataggtaagatcgttacagagggtgacggtgagatgaagggattgaatgagcacataaacctttataatcaacagagagggcgcgaagataggttgccgttgtttcgccccctgtataagcaaatcttgtcagaccgcgagcagctttcgtatctgcctgagtcgttcgagaaggacgaagaactgctgagggcgctcaaagagttttatgatcacattgcagaagatattttgggacgcacgcagcagttgatgacctctatatccgagtatgatctgagtcgcatctatgtccgcaacgactcccagttgactgatatatcgaaaaagatgctgggagattggaatgcaatttacatggcacgcgagagggcctacgatcatgaacaggctccgaaacggatcacagcaaaatatgaaagggatcgcataaaagcgttgaagggagaggagtctatcagtctcgcaaacttgaattcgtgtatcgcattccttgacaatgtgcgggattgtagggtcgatacttacctctctacacttgggcagaaagagggcccacatggtctgagcaacttggttgagaatgtctttgcatcctatcatgaagctgagcagttgttgtcattcccgtatcctgaggagaacaacctcattcaggacaaagataatgtcgtgttgattaagaatctgcttgacaacataagtgatcttcaacgcttcctgaagcccctgtggggtatgggtgatgagccagacaaagatgaaagattttacggtgagtataattatattaggggtgcgcttgaccaagttatcccactctataacaaagtcaggaactaccttacgaggaagccatactctactagaaaagtcaagcttaattttgggaacagtcagctgttgtcaggttgggacagaaataaagaaaaagacaattcttgcgttatactgaggaaggggcagaatttctacctcgccataatgaacaatcggcataaacgctctttcgaaaataaggtccttcctgagtataaggaaggagagccttatttcgaaaaaatggactacaagtttctgcccgatccaaataaaatgcttccgaaggtttttctctcaaagaaaggtattgagatttataaaccatctcccaagttgcttgaacaatatggtcatggcacacacaaaaagggtgatacgttttctatggatgatcttcatgaactgatcgatttctttaaacattctattgaagcgcatgaagattggaagcaattcggtttcaaattttctgatacggcaacttacgaaaatgtttcgagcttttatagggaggttgaggaccaaggttataaactttcgttcagaaaggtgagtgaatcgtatgtttacagtctgatagatcaaggaaagctctatctttttcaaatttataacaaggacttttctccgtgttcaaaaggaactccgaacttgcataccttgtattggcgcatgctttttgacgagcgcaacttggccgatgtgatttataagcttgatggaaaagccgaaattttcttccgcgaaaaatctcttaagaacgatcatcctacccaccctgccggtaagccgattaaaaagaagtcaaggcaaaaaaagggtgaagagtcactctttgagtacgatctcgtcaaagatcggagatacacaatggacaaattccaatttcatgttccaattactatgaatttcaagtgctctgctggttcgaaggtcaacgatatggtcaatgcacacattagggaggcaaaggatatgcacgtgatcggaattgatagaggggagcgcaacttgctgtacatttgcgtgattgactctagaggtactatcctggatcaaatatccctgaacacaataaatgacatcgactaccacgatcttctcgaatcccgcgataaagaccgccagcaggagagacgcaactggcagactattgaaggcatcaaggaacttaaacaagggtacctgtcgcaagcggtccatcggatcgctgagctgatggtcgcttataaggccgtcgtcgcgttggaggacttgaacatggggttcaaacggggcagacagaaggttgaatctagtgtctaccaacagtttgaaaaacagttgattgataaactgaattacttggtggacaagaagaagcgccctgaagatatcggcggactgctcagagcctatcagtttacagctcctttcaaatcgttcaaagaaatgggaaagcaaaatgggtttcttttctacataccagcttggaatacatcaaacatagatcctacaacaggttttgttaacctgtttcatgcgcagtacgaaaacgttgacaaagcaaaaagcttctttcagaaatttgattctatctcctacaacccaaagaaggactggttcgaatttgccttcgattataagaattttactaagaaagcggagggatcgcgctctatgtggatcttgtgtactcacggatcgagaattaagaatttcaggaattcacagaaaaatggacagtgggacagtgaggagttcgctttgactgaagcatttaagagcttgttcgttaggtacgagatagactacacagccgacctcaagacagcgatcgtcgatgagaaacagaaagatttttttgtggatctcctcaagctctttaagctcacagtgcagatgcggaactcctggaaggaaaaggacctcgactaccttatttcgcccgtggctggcgcagatgggagattcttcgatactcgcgagggtaacaaatcccttcctaaggacgcagacgcgaacggggcttataacattgcgttgaagggattgtgggcgctgagacagattcgccagacctcagaaggtggcaaacttaagctcgctatttcgaacaaggaatggctgcaattcgttcaggaaagatcgtatgagaaggat

SEQ ID NO: 10 - DNA, 3930 bp, Artificial Sequence
  synthetic
atgaaggaattcacaaatcaatattctttgacaaaaacgctccggttcgagctcaggcctgtcggcgaaaccgccgagaaaattgaggatttcaaatctggtgggctcaaacaaactgtcgaaaaagacagagagcgcacagaagcctataaacagcttaaagaagtcattgattcgtaccacagggactttattgaacaagcatttgctcggcagcagactcttagtgaagaagatttcaaacagacctatcagctctacaaggaggcacaaaaagaaaaagacggagagacgcttacgaaacaatatgagcatctccgcaaaaagatagctgctatgttttccaaggccactaaagagtgggccgtcatgggcgagaataacgaattgattgggaagaacaaggagagcaaattgtatcagtggctcgagaagaattacagagcaggccggattgaaaaggaggaattcgaccacaatgctggtctcatcgagtacttcgaaaagtttagtacatactttgtgggatttgacaagaaccgggctaacatgtacagcaaggaggcaaaagccaccgccatcagcttccggactatcaatgaaaacatggttaagcacttcgacaattgtcaaagacttgagaagataaagtcgaaatatccggacctcgcagaagaactcaaagatttcgaggaattcttcaaaccaagctatttcatcaattgcatgaaccaatctgggatagattactataatatatccgcaatcgggggaaaggacgaaaaggaccaaaaagcaaatatgaaaataaatctttttacgcagaaaaaccacttgaaggggagcgacaagccaccattcttcgctaagctctataaacaaatcctgagtgacagggagaaaagcgtggtgatagacgagttcgaaaaggattccgaacttaccgaagcgctgaaaaacgttttctcaaaggatggcctcataaacgaagaattctttactaaactcaagagtgcgctggagaatttcatgcttcccgaataccaaggacagctttatatccgcaacgcttttctgacgaaaatcagtgcgaacatatggggttccggttcgtggggtatcatcaaggacgcggtcactcaggccgcggaaaataacttcacgcggaagtcagacaaagaaaagtacgctaaaaaagatttttattccatagcagaacttcagcaagccatcgacgagtatatccccactctcgaaaacggggtccagaatgcctcattgatcgaatacttccggaagatgaactacaaaccaagaggttcagaagaagatgccgggttgatagaagagatcaataacaatctgagacaagccggaatcgttcttaatcaagctgagttgggaagcggtaaacagcgcgaagaaaatatagaaaaaataaaaaatcttcttgatagtgtccttaacctggaaagatttcttaagcccctctatttggaaaaggagaaaatgaggcctaaggcggcaaatctcaacaaggatttctgtgaatcctttgatcccctgtacgaaaagctcaaaacattctttaagttgtataacaaagtcaggaattacgcgacgaagaaaccctactcaaaggataagtttaaaatcaacttcgataccgccacgcttctctatggttggtccctggacaaggagacagcaaacctcagtgtcatctttcggaagagagagaaattttacttgggtatcataaataggtataactcacagatatttaattacaaaattgccggcagcgaatcagagaaagggcttgaacggaagagaagtctgcaacaaaaagtgctggccgaggaaggtgaagactattttgaaaaaatggtttaccatcttctgctgggggcttcaaaaactattcccaaatgctctactcaactgaaagaagttaaggcgcatttccagaaaagctctgaggactatatcatacagtccaagtccttcgctaagtcactgacccttactaaggaaatatttgatcttaacaatttgagatataatactgagacaggggagatatctagtgaattgtcggacacgtatccgaagaaattccaaaagggctacctcacacagacaggcgatgtgtctggttataaaacagccctccacaaatggattgatttttgcaaggaattcctgagatgctacagaaatactgagatttttactttccactttaaagatacgaaggaatacgaatccttggacgagttcctcaaggaggtcgacagttctggttatgagattagctttgacaaaatcaaagcgagctatattaatgaaaaggtgaatgcgggagaactgtatctgttcgaaatttataacaaggatttttcggagtattctaaggggaaacctaatttgcacacgatatactggaaatcactgttcgagacacaaaaccttctggataagaccgcaaagctcaatggtaaggccgaaatattctttagaccgagatccataaagcacaatgataaaatcatccatcgcgcgggagaaacgctcaagaacaagaatccccttaatgagaaaccaagctcccggttcgattacgacattactaaagatagaaggttcaccaaagataagtttttccttcactgtcctatcacattgaacttcaagcaggataaaccggtcagattcaatgagcaggtgaatctctaccttaaggacaatcctgacgttaacataatcggtatagaccgcggtgagcgccatcttctgtattataccctcataaaccagaatggcgagatcttgcagcaaggctccctgaacagaataggtgaagaagaatctcggcctaccgactaccatcggctgcttgatgaaagggaaaaacagcggcaacaggcgagagaaacttggaaggctgttgagggtattaaagatctcaaggctgggtatctctcgcgcgttgtccacaaactcgcgggtctgatggtccagaataatgcgatagtcgttttggaggacctgaataaaggttttaaacggggtaggttcgcagtcgagaagcaagtgtatcagaattttgaaaaggctctgatccaaaagcttaattaccttgtcttcaaggaagttaattcaaaagacgctcccggccactatttgaaagcgtatcagttgacagcgccatttatatccttcgaaaagcttggcacccagtcggggttcttgttctatgtcagagcatggaacacctctaaaatagaccccgcaactgggttcaccgaccaaataaaacccaagtacaagaatcagaaacaagccaaggatttcatgagtagttttgatagtgtgaggtacaatcggaaggaaaattactttgaatttgaggcagacttcgaaaagttggctcaaaaaccaaaagggcggactagatggaccatttgctcatacgggcaggaaaggtatagttattcaccaaaagagcggaaatttgttaaacataatgtgacccagaacctcgcagagttgttcaattccgaaggaatctccttcgacagtggacaatgcttcaaagacgagattctgaaagttgaagatgccagctttttcaagtccatcatctttaacttgaggttgcttctgaagttgaggcatacatgtaaaaacgcggagatcgaaagagattttattatatcacccgtgaaaggcaataatagtagcttttttgattctcggatcgcagagcaggagaacatcacatccatcccgcagaacgcggacgccaacggggcttataatatagcgcttaagggcttgatgaacttgcataatatctctaaggatggtaaagccaagttgatcaaagatgaagattggattgaattcgtccaaaaaaggaaattc

SEQ ID NO: 11 - DNA, 4425 bp, Artificial Sequence
  synthetic
atgatttacaatgttaaacttaaaaagctcaccaattcttccgatgttttctccttgttcagccggaagtactcactttcaaagacgttgaaattcgagctgaaaccgacggcggaaacgaaacagcatctgcaggacttcatcgtgtctgacaccaaaagagcgaaagagtacaaggagctcaagaagatcatcgatgagtttcacaaagactatattgagttgaccttgtcccataagaacattctcgacaaagataagctgaccgatttttgtaaactgtggagcgataataacctgcttgacgaaattaaggagaaacacaacctgaaagagaagactaaagaagaggtgatcaagaagatggaacagcaatttagaaaggacattgtcgagcaattcaaaactctgaatccccttttggagaggttttttaaaacagcagacgaggagcagctgttgggttttcttaagaaacttgacaataaactttggacaaagtttaaggagaagaaggagaaagcgctccttgacgagtacctgcaagaacctatcaagcagaagaatatcgagaagagtatcctgttctcttcggagcttatcaagtatctcctccccgtttggctgaaaaactctcaactgacagacaaggaggacaaacagaagattatcaaaaacttcgggaggttcacgacttacttgaccggtttcaacgaaaacaggaagaatatgtattcggtggaagaacaaggcaccgcgattgcccatagaataatcaatgagaacctgatgaagttcctgggcaaccttcaggcatatgaaaagataaaatcgagccacccggaattgcaacagtcattcaaaaatatgaaaagcgactttaaggaagagtttgattatttctccctggagaacattgaagatctcttcaagccagaattctttaattcatgtctctcacaaaaagagatagatttctataatacgctgcttggcggcaaaacggtggaggatggcaaaaaattgcaggggattaatgaatatatcaatctgtacaggcagaaaaaaaaatcggaggacatagatataaaaataaagtacagtaacaagaacttgcctacaatggagctgctctacaaacagatactttccgaccgcgaaagtcacagtttcattctggctgaattcgaaaataagaaggagttgcttgaagcgctgaaatccttttggtcagtccttttcgaagaaaaaacgtatcaagactactttcatactaaaaagaagacaagcctgtggaataatcttcatagcctcttgacggggcggagctattacgatttggaaggagcctacttcaaaagtagtgagctcaataaactttcccataatctgttcaaagattatagaattataactgaagccctcaatgagaattacgacaaaattaaggtcactctcgaatcgtacaagaaggtgctgaagaaagaagacaagaaagagttcgagaaaaacatcaagtctttgaaaggctttcagacggaatggaaaaagatcctgtccttcgagaaaaacaaagaggagcttaaattctcggacgagatattggcgtcatatttcgaatttcccatcgataaaaagacgaaaaagaaaaaccaaaaagatttctactcgcttcaggaaatcaaagaccacatcgaactctattcgaaagagtcagatgaactgaaggaagatttggataatcttaagaaaaagttgaaggattacaaaaaagacaatattattagttcgtggtttaaataccaattcgaaagtaaacagaatctcgacttcttccttcaaagaaaccacgaaaaagaaaatgaggacaaacgcgagcagaacaagaactctttgttctctcacatcgaaaatcattatcaaaagatccagaatctgtctcttgatgagaaagagtttaagaaggaagaggtggaagatataaaattttttcttgacctcatcctgcatgttttgcatctgatgaagccgttctaccttgaagggaaagccagtaacttgttggacaaaggcatcaataatgaaatcgaagtcttgtacaagaagctcaaaccaatagagaaactgtataatcagacccgcaactacatagcgaaaaagaagagcagatataacaaagttaagataaatttcgaagactcgacgctgctcgatggttgggacctgaataaagagaaagacaatctggcggtgctgcttaggaagaaagacgcggtcataggttggaaatactacttgggtgtgatgaataagactacgaaagacttgtttgactatcacattaagttggacgactccgagaaagttaaacaaaaaaaggaggagctccaagacctcatcctccatagggagaacgatggaaatttctacgagaaaatgaactacaaactccttcccgacccttccaagatgttgcctaaggtctttttctctaagaagaatctggactcattcaagccctctgaggaaatagtgcgcatcagggataacaaaacttacgcaaaaaacggggggcaagatttctctaaagcagattgtcataaatttatagacttttataaggaatccctgaaaaaacataaggagtggaatgacttcttcaagttcaacttcagccccacatcgcagtataatgacattagcgacttttttcaagaggtcaagaaccagggatataatcttcagttcgataagataaagtcatcttatattgaagagaaagtgaagacgggagaacttttcctttttgaaatttattccaaggacttctcaactaagtctaagaacagaaagaacagtaaagataatctgcacaccatttacttcaaagggttgtttgaaaaagaaaacctgaaggacacggtgctgaagctgaatggaaaagctgaaattttttacagaaaagcgactaaaaagttcaacataacgcacaaaaaaaacacagaactggaaaacaaaaataagaataatccaaacaagcatagtattttcaactatgatttgatcaaggataaaagattcaccgaagacaaatttttttttcactttccgatctccttgaacttcaatagtaaagggatgaaaagctacctgtttaatcaagaggtgcttaaatgtctgaagggaaataaggaagtcaacataattggtattgacaggggtgaacggcacctcgcgtactatacaataatcaaccaaaagcgcgaggttctctcacagggatctttcaataaaatggagagctcctacaaggataactcgggtaaggaagtgaaaatcgagaaagactatcacgagcttcttgagtcaaaggaaaaagaacgggacaagtcgcggaaagagtggaataagattgagaatatcaaggaattgaaatcagggtatctctcttatctggtgcataaaatctcaaaacttatgatcgagcacaaagcaattgttatcttcgaagacctcaacctggggttcaagagaggacgcttcaaattcgagaagcaagtctatcaaaagctggagaaggcccttatcgataaattgaattatctggtcttcaaggataagaaatcaagcgaacttggggggtatctgaatgcgtaccagctcactgcgccattcgaaagttttcaacgcatggggaaacaaacaggttacctcttttatgtgccagcgtactacacgtcgaaagtgtgcccactgacgggatttatcaacctgatctaccctaagtacaagaatgtcaaagagtcccagcagttttttgaaaaatttgatagaatatatttcgacaaaaacaaaaactatttcgtcttcgaataccaagataagaaggtgaatccctcgaaaaagactgagagcatagaaactttgtggaaggtgtgcacacacggggaggaacggtataaatgggatgttaaggatcggaaaatgatagaagttaacgtgactgaaaaccttaagaaactctttgaggaacataaaatagaatacagacaaatcgccgacctcaaatctaccataaccaagcaagaaaaaaaggatttcttctctaagttgatagatggactgaaaattacattgcagctgcgccatatcaacccagattcgaaggacgagaaggaaaaagatttcatactgagcccggtcgctgacgaatccggtaggttcttcgactcacgcaaggcgaaagaaggggaacctaaaaacgcggatgcgaacggagcctatcatatagcattgaagggtttgcggactcttgagaacatcacctcggacaaaaaggacaagttgaaattgcaggcaattactaacaaagattggttctcgtttcttaaggagaatagtaataagaagatacctaaagtgggt

SEQ ID NO: 12 - DNA, 3183 bp, Artificial Sequence
  synthetic
atgaacaaagcggactctgtcttctcgaactttacaaaccaatactccctgtcaaaaaccttgaagtttgagcttaaaccgcatccccttactaagtctcttgtcgaagtcgttaataaggacaaagaaatagacagactgagcacaaaagagatgaagcctatgctcgatgcgttgcatgagaaatttatcacagaatccttggagaaaatgattttttctatctccgacttggagcaactcgaaaggcacctcgacgatcttaaagtgctcaggcaaaagctgaagaacttgaacaataataaggataaaaagaaaaaagaaattgatagcatacagaaggaaatcaaaaatttggaagataagaagagcggaaagatacctaagcttcagcagaaactgcggcaagtgattgttgatcagataaacgaaataggcgacgagtggaaggaaatttatacaaaggaaggcattaagttcgaaaaggataatggaaataagaaaaaacaaggctatgaaatgcttactaccaagggggtcttggggatcttgaaatcagagtactcccagtaccctgaaaaaataaaggccatcagcgagtttgaccaattcttcacttatttcaaagggctgaatgacaatagggcgaactattataccgacgaacaaaaagcgacaggggtggcaaatcggataatcaacgtcaatcttaatatattccttaagaataaaaatgatttctccctgttcctcgagaagctccctcaactgaaggattatgagacgtcattcgctctgagtaattttcaaaaatgcttggcacaggcggacatcgaagcatacaacgaaaaagtcggggaagtgaaaaaactggtgaatctggaatacaatcagaaggtcgctgacaagaaagacttgctgaaaggactcaacaagcttcagaagcagataggttgtaagataaaacaggagagggaacagcttgagaggggtgagtccatctaccccaagtacctggaaaaggtgggactgggctttcatataactaaaacatcagatgataaatatcaaatttgggagtctcttgactacctgaacgaccagttgattcctcaagtcaaaaagcttaaggataactacgagtcattttttgagaactggcaggattacaaacttaatgaaatttggtttaggcgcgaatcacttaatacaatatcgggcagatggttcggaggcaacaattggtttgttttgaccaaggccttggcttattttggtacgggaaagatgaataaaggtgaatataagcccgaccaattcatcagcttgcttgaattgaaggaggctatggaggcactggaaaatggagttgactttgacatcaagaagccaaacaaaaagactaaagaaggccaaaccgaagcaataaaagatgaaataacgaaacagtatttttataaacccgagaacctgttcagggataagtataaagatatttacaagaataagacgctgtttgttgcgttccttacgatctggcgctacgagctgaaacatcgcttcaaagaaattgagttttaccttgatgacttcaacattcaaaagaaaacgcctttcgataagggcaataaggaacacgttaagaccgttttcaacttgatgcaagaaggttatctccccatactccaaatgacgaagtatcattcacttgaaaaaaagagcgagatcgttccaggttacaatacggaaaataaattttatgagactcttaacgacttctgggaagacaaccccataaatcaatacagatcagcattcgaggctaccttgactcaaaaaccctacagcgaggataagataaaactcaattttgactgtgggtcactgttgggaggttggagcccgacatatgaggcctacggaagtctcatatttaaaaaggataaccggttttatttggtgttgataaagggtaccggtctgtcagacgaggagatgaaggggttgtatcagtctattgatcgcaaaaatagtatcgaacggttgatctataacgcccagaagctcgactttaaaaattgcccacgctggtttataaattctataggagacagcaaggcaccggccgtcaaaaagtataacctgccggtggagagcatatgggaagactatcaaatttacaggaagttggataaaaacggaaaggataagtaccttgaagacaataagaatttccggttccggttgatcgattactataaacattgtcttccgttccatgaatcacttaaggagttcaagggcaattttagttggaaagatacgaaggactacaacaacctcaatgagttctacacggacgcggttaattcctgctataaaatttcatgggagaacattaacttcgatgccctcaaaaggctcgtgcggaacgagagaatatatctcttccagatctataataaagacttcgaaatagacaccgagatcggtgagaagaaatacggcgagagctacaacacaaaggagaagctcagaaaggcacaggggaaccccaagggggtcgtcaatctgcacacacagctcttcttcgaactcctcaagcccgaaaacataaagtatcttaagcttatgggcggtggagaaatcttttttcgcgaaccttccaaagagaaaagttataggttcgacaccgggcgccgggaagttcttaaggctaagcggtattacgaagaaaagtattttctgcacttccctgtcgagattaagaagaccaaggaaagtagcgaaagctttaatcagaagatatataagaccttgactcagaatcataaaccttgtaagataataggcatagacagaggagagaaacatctgctctattactctgtcatatccatcaacgagagtggaggaacaaaaatagaggatcaggggtcacttaatcgcgtcaagacaaagtcgccagtggacgagaagaaactcgagtgtgagtatgatgaaaaaggaaaactccagaaggtcgatcttgtgtcaaccggtaaaaaagtcaattatgtcgattaccatctgttgctggattactatgaaaagaaaaggaatctcgcccgcaagagctgggaagtcataggtaagattaaggacctgaaggagggctatctgtcccaggttatttacaaaata

SEQ ID NO: 13 - DNA, 3939 bp, Artificial Sequence
  synthetic
atgtccaagctgcacttcgccacgcttcttactggcgctataatgggcgtcgttcgcatcgacgacaaactcaaaaagctcttttgggatctctatagccttaggaaaacgctgaggtttgagcttaaaccccaaggtaagactcttgagcatatcgagaataagggactcataagtcaggacgagcaaagagcggagagttataagaaaatgaagaagacgataaatggattccataaacatttcatcgaaatcgccatgtctcaggtcaaacttacaaaactgaacgaatttgcggacttgtacaacgcatctccagaacggaaaaaagaggacggcttcaaaaaggccttcgaaaagattcaggcagatctgagaaaagacatcgcaaaggcatttaacgttggcgaggcgaaggaaattttttcaaaaattgataaaaaggagctggttaccgaactcttggagaaatggattcggacgcaggaggaagacctctactttgatgaaagatttaagacctttacgacttacttcggagggtttcacgaaaatcgcaagaacatgtactctgataaagagcagtctacggctatcgcgtacagacttatacacgagaatttgcctaagtttcttgacaatattaaggttttcgaaaggatcaaggagatctccgagttgtacaaaaacttcccggcattgtataaggaaattaaagaatacctcgatatcgaacaaattgacgaagcatttgagttgtattactacaataagattttgacacaaaaacagatcgatgcttacaacttgatcattggtgggatgaccgcccaagaaggcaagaaaaagatacaaggtctgaacgaatatataaatttgtataaccaaaagcaagacaagaaggacagaatccccaccctgaaacaactgtataaacaaattttgagtgacaaagaatctgtgtcttttctgcccgatgagttcgaggacgatgctgaagaaacggcatcacagaaggtccttaatgcgatcgaagagtactatcaagcgaacctggtgtcgtttcacccatcaggcaaggatgaggcggaaaacgtgttggaaaaaataaaggagttgctcacttctctcaaggattatgatttgaacaaaatatatcttcgcaatgataaggccataactgatattagcaaaactctttttggcgattggaacataattaaggcagcgctcgagtttgcgtttttgcaaacgttggaaacgggaaaaaagggactctctaaaaagcaggagaaggaaaaggaaaagtaccttgaacaatcctatttctcgattgcggagattgagaaagctctttttgcatataaaaacgaagcggaggtccttaaagatctcaaagaaaatacgacccccatcgccgattatttccatacccacttccttgcaaagaaaaaagaagagtcagacaaggagttcgacatgattgcgaatattcaagcaaaatatagttgcatcaaggggatcctcaataccgactacccgaaggaaaaccgcttgcaccaggataaaaagaccattggtgatattaaggcgttccttgacagcctcatggagcttctgcacttcgtcaagcaaatcagtctcccctcggacactcctctcgaaaaggatgaaatcttctactcacaatttcagccctggtatgatcagcttcaactgctgatccccctgtacaataaggttagaaattatgcgtctcaaaagccctacagcatcgagaagtttaaattgaattttcaatctagtacacttgcccaggggtgggatttgaataaagagcacaataaaaattacagcctcattttcaaggagaacgactcgttctttcttggtatcattaatattgatgacgacaaggacgttctgcggaaagataaaaggccggagatctttgttgatgattctccattgaaaaaaatggtctacaaagacttgggggacattaagcgccagcttcctcgcatcgccttcagcgataaggccaaaaaggacgttgaggatattggctggaatcagaacatagaagcaattaagaaagagttcgataaattccaagacgccaaaaaaaacgataaggacctctggaaaaacgagtttgatagaaataagctgtccgaactgatagactactataagggggttctcaagaatcattctgagcgctatgaggatacctacaagctcgagtataaatccacggagtcttataaaaacctcggtgaattcttcgaccatatagcctctcagacatacaaggtgacctttgttggaattgacaaaaattacataaatcaactggtcgaccagggaaaactgtatctgtttcaaatttataacaaggacttttccaaatattcaaagggaaagccaaatctccacacgctgtattggaaagctctgttcgatccggaaaaccttaaaaatgtcatgtacaaactgaatggcggagccgaattgttttacagaaagaaatcaataaaggacttgacgatccataagtcgaacgaacccattaagaacaagaacccgaacaatcccaaaaaagagtcgttttttgagtatgatattgtcaaggatcggaggtactcggttgataaatttcaatttcacgttcctattacgttgaactttaaagcaacggagaaaaaatacataaacaacacagtgcttgggtttcttaaagataatcctgacgttaacatcatcggtatagataggggggagcggcatcttttgtacctgacgttgattgaccagaaggggaaaattctcgagcagagatcgttgaacacgatagttaataatcgcgttaatattaagaccgactatcacaaactgctccacaacagagagaaggagcgggataaagctagaaaagattgggggacaattgagactatcaaagaactgaaggatggctatatatctataatcgtgcatgagatatcgcgcatgatggtcgagcacaacgctatagttgttatggaagatctcaacttcggctttaagcggggtcggttcaaggttgagaaacagatataccaaaaatttgagaagaaactcatagataaattgaactatctggtcttcaaagataaagcaaataatgagacagggggactgtacaaagctctgcagttgacttccaagttcgagagttttactaagttgggaaaacagtcgggatttctgttttatgtccccgcttggaacacaagtaaaatcgatccaactacaggattcgttaacctcttcaatacgcgctacgagaatgtcgataaagcaaaaacattcttcaacaattttgagaacatacggtacaacacggataaagataactttgagtttgaggtcaagaaatactctttgttcaacagtaaagtggatgacacgattcaagattgggtcatatgcacccacggggagaggatcaagacgttccggaatagtgagaaaaataaccagtgggacaataaagagattgtgcttacagaggagttcaagaaccttttcaaggagtataatataaattataaaagagatctgaagaatcaaataaattcacaaacaaacaaggatttctttgagcaactcttgaacctgtttaaatacactgtgcagatgagaaattcaattactaattcagagactgattacctgatttcgcccgtcatcaactcagagggtaagttttatgattcgaggaatgcaggcgacgaacttcccaaagacgcagatgccaatggagcttaccacatagctaagaaaggtttgtgggttctgcagcaaattaatgctcatgagggggaagattggaaaaagttgaaattggcaatttctaataaagaatggttgcaatttgtgcaggctcgcgcaaag

SEQ ID NO: 14 - DNA, 3855 bp, Artificial Sequence
  synthetic
atgaagtcgtttgattcatttaccaacttgtatagtttgtcgaaaacgctcaaatttgaaatgcgcccagtcggcaatacacaaaaaatgttggacaatgccggggtctttgaaaaggacaagcttattcagaagaaatatgggaagacgaaaccatactttgaccggttgcaccgggaattcatcgaagaggcccttacaggtgtcgagttgatcgggctggacgaaaatttcaggacgctcgtggattggcagaaggataaaaaaaataatgtcgcgatgaaggcgtacgagaactcactccaaagattgagaacggaaatcgggaaaatatttaatctcaaggcagaggattgggtcaaaaacaagtatccaatccttggtttgaaaaacaagaacacggacattctgttcgaggaagctgtttttggcatactcaaagcgcgctacggggaagaaaaagatacgtttatagaggtcgaagaaatcgataagacggggaagtcgaagattaaccagatcagtatctttgacagctggaaggggtttacggggtattttaaaaaattttttgagacgaggaaaaatttttataaaaatgatggaacgtccactgccatcgcgacgcggataatcgatcagaacctgaagcggttcattgataatctgtccatcgtggagagtgtccgccaaaaggtcgacctggcagaaacggaaaagtcattcagtatcagtctttcgcagttcttctcaatcgatttctacaacaagtgtctcttgcaagacggtattgactattataataaaattattggaggtgagactctgaaaaacggcgaaaagctcataggccttaatgaactgatcaaccaataccggcaaaacaacaaagaccagaagataccgttttttaaattgctcgacaagcaaatcctctctgagaagatactttttctcgacgagattaagaatgatacagagttgatagaagcacttagtcaatttgccaagactgctgaagagaagaccaagatagtcaaaaagctgtttgcagacttcgttgagaacaattccaagtacgatctcgctcaaatatatatctctcaagaggcctttaacaccatcagtaacaagtggacatcagagacggaaacgtttgcgaagtatctgttcgaggctatgaagtctgggaaacttgccaagtacgagaagaaggacaactcctataagttccctgactttattgctctttcgcagatgaagagcgctctcttgagtatatcactcgaagggcatttctggaaggagaaatattataagataagcaagtttcaggaaaaaactaattgggaacaatttctggctatctttctctacgaattcaacagtctcttttcagacaaaataaacactaaggacggagagacaaaacaagttgggtattacttgtttgcaaaagacctccataaccttattctgagtgagcagatcgacatacccaaggattccaaggtcactattaaagatttcgccgattctgttttgaccatttaccagatggcgaagtactttgcagtcgaaaagaagcgcgcatggttggccgaatatgagctggactccttctacacccagcccgatactggatacctccaattttacgataacgcctatgaggatatagtgcaggtctataataaattgaggaattatctcaccaagaagccctacagcgaggaaaagtggaaactgaacttcgaaaattccacgcttgccaatggttgggataaaaacaaggaaagcgataatagtgccgtgattcttcaaaagggcggcaaatactatctggggttgattacaaaaggccataataagatattcgacgaccggtttcaagaaaagtttatcgttggtatcgaaggtgggaaatatgaaaaaattgtgtacaaattcttccccgatcaggcgaagatgtttccaaaagtgtgcttctccgcgaaggggcttgagttctttcggccttcagaggagatactccggatttacaacaacgcggaatttaagaagggggagacttacagtatcgattcgatgcaaaagctgatcgatttttataaagactgcctgactaagtacgaaggctgggcttgctacaccttcaggcatctgaagccaacagaggaataccaaaacaatatcggtgagttctttagggatgtcgcggaagatggctacagaatcgacttccaaggaatttcagaccaatatatacacgaaaagaacgaaaaaggggaactgcacttgttcgagatacataataaggattggaacctcgataaggccagggatggaaagtcgaagacgacccaaaaaaacttgcacacattgtattttgaaagtttgttctccaatgataatgtggtgcaaaatttccctatcaagctcaatggccaggcagaaatcttctataggcccaaaactgagaaggacaagcttgaatccaagaaggacaagaagggaaataaagtgatcgaccacaagcggtacagcgaaaacaaaatattttttcacgtccccctcactttgaaccggacgaaaaacgacagttacagattcaacgcgcaaattaacaacttccttgcgaacaataaagacataaacattattggagtcgatcgcggcgaaaaacatcttgtctactacagtgttatcacgcaagcgagcgatatcctggagagcggctcacttaatgagctcaatggagtcaattacgcggagaaactcggaaagaaggcagaaaatcgggaacaggcacgccgggactggcaagacgtccaagggatcaaggacttgaaaaagggatatataagccaagttgtcagaaagcttgcagatctggccattaaacacaacgccataattattttggaagatcttaatatgagatttaagcaggttaggggcggcatagagaaatccatatatcagcagttggagaaagcgttgattgacaaactttcgtttctcgttgacaaaggtgaaaaaaaccctgagcaagccggccatctgttgaaagcgtaccagttgtctgcacccttcgaaacgttccagaagatggggaagcaaaccgggataatattttacacacaagctagttacacaagtaagtcagatcctgttacgggatggcggccccacctgtacctgaaatatttctcagccaagaaggctaaagacgatattgcaaaatttacgaaaattgaatttgtgaacgatcggtttgaactgacgtatgatatcaaggattttcaacaggcgaaggaataccctaataaaacggtttggaaagtttgcagtaacgttgaacgcttccgctgggataaaaaccttaatcagaacaaaggaggatatactcattacacaaatattacagaaaatatccaagaactctttacgaaatacgggatagatataactaaggatcttcttacgcaaatttcaactatcgatgaaaaacagaatacgagcttttttcgggactttatattctactttaaccttatatgccaaatacgcaacacagatgatagtgaaattgccaagaagaatggtaaagatgactttatactgagtcctgtggagccttttttcgatagtcggaaagataacgggaataaacttcccgagaatggggatgacaatggggcgtacaacattgcaagaaaaggcatcgttatcttgaataaaatatcacagtactccgagaagaatgagaactgtgagaagatgaaatggggcgacctttacgtctctaatatagattgggacaacttcgtcacacaagctaacgcccgccat

SEQ ID NO: 15 - DNA, 3867 bp, Artificial Sequence
  synthetic
atggagaacaaagaactgtttaactcatttactaagaagtgccagctttccaaaacgcttcggtttgaattggtgccccaaggcgagacagaaaaattcatcaagaaaaaagggttgttgaagcaggataaagacagagctgatgactacaggaaagctaaaaagcttattgacgagtaccacaaagacttcatcgaacacgccttgtctggtaaacagcttaagaagcttcaggcttactacgacgaatttacggcactgctgggtaaaccggctaaggaacgggatacccgggttctgtcgaatatttccgaagctcttagaaaagaaatcgctggatggttgaaagataatcccatcaaagatgagaagaagctcataaaggagatcgtgccgaacttccttaaaagtaaagacagaaacgacgatgccgctcttgttctcaaatttaagggatttacgacttacttcgggggtttcaatgagaataggagaaacatgtacagcgcggaagagaagagtactgccatagcctatcggatcgtccatgagaatcttccaaaatttatttccaatatgaaaactttccaaaggctcacggagaatcataagatagacttccttgaagttgagacacaaatgaacgacgaactggacgggaataaactgaaagacgtcttctcaatcgattactttaatcaatgcctgactcagtcgggtatcgatcgctacaacactatacttggaggaaagacagacagtcgcggtgcgcagatccagggtgtcaatataaagattaacttgttcggacagaagaataatttgaaatcaaaggacgcgcccgttctcgcgcggctctataagcaaattttgtcggataggacttcggccagctttttgccagaaaaatttgagactgctaaggaactcatcgcatcaataagggagttttacgagaaggccatcaatggatttattaaaaacaatgagagaaagaatgtcatagatgagatcgagtcgctgttcaacaaaaagctcaacccgaccgattgcgatctctctcaaatctatatacgcaatatttttatatcggccgtgagccagcagatttttgggtcctatagtgttatcacctctgctctgaatgcctttatagacaagacatataaaaccaagaaagaaaaggaaaagcagcaaaagaaatcctatttcggtatagctgaaattgaagcagctcttaaggcctatctgcaagaaaccgaaatagagaacaaggaaaagagggaggaagtccttaaacacgcaaaccctgtcttgcagtaccttaattcccttacaacgaatattgaaattaacaataaaaaggaaaaagctaacgtgattgtgaggtttcgccagcagcatgaggctgtgcaatccgttcttaacaaggagtatggcaacaatagtgaactgatccaggataaaaacgcggttgctgcaattaaggactacttggacagctgccagcatatccttcacttcgttaagcctcttttcgtggttcctccaaaaaaggaaaacgccgaactgccagacaaggacgccttctattccgagttcgatgagcttttcgaacaactcaagtccgttacacccttgtataacatggttcggaatttcctgacccagaaaccttattctaccgaaaagatcaaactgaatttcgaaaatgtgtccctgttgtccggctgggatgtcaataaagagacagataacacaagcgttatatttagaaaagatagcttgtactatcttggtattatggacaagaaacataataaggtgttccacgacataccggcctcgtccgataatgacttctacgagaagatgaattacaagctcttgcccggggcgaataagatgctgcccaaggttatttttagtgatcggtggatcgaatatttcaatccaagtaaggaactcatagaaaaatataagaagggcacacacaaaaaaggtgaaattttcagcctgtccgattgccacgcgttgattgatttttttaaaacctcaattgcaaagcatgaggattggaagcagttcaattttcaattctctccgacgaagacctatcaggatataaacgaattctaccgcgaggtcgaacaccagggatacaaaataagtttccaaaacgtgtcagcgaaatatatacatcaattggtgtctgaaggaaagctgttcttgttcaaaatttacaacaaagatttcagcccctattctaagggaaaaccgaatctccacacgctttattggaaggccgtgttcgacaaagaaaacttgaaagacgttgttgtgaaacttaatggagaagctgaggttttctaccggcctcagtccattccgaagaagagcaagatcgtgcacaaaaagaacaaacctatagtcaacaaaaaccccaataaccccaaaaaacagtctctgtttaaagaatatgacattgttaaagataaacggtacaccgaagacaaataccactttcacgttcccatcactctcaatttcaaggaggctaaggttcccagtgagaacgacagaaagaaagccttcgcatatacctttaataaaggtgcaaatgaaacgctcaaggaaaattttccaaatatcatagggatagacagaggagagaggcaccttgcgtactatagccttataaattcaaagggagaaatcctggagcaaggctcgtttaatgttattaagaacgaattcaatggcaccaagcacgaaacggattactacgcttcacttgttgaaaaagagaaagccagagatgcagcaagaaaaagttgggatacaatcggcaaaatcaaggaattgaaagagggctatctcagccaagttgttcataagatagccagaatgatgatcgaacataacgccatcatagtcctcgaagacctcactatgaagtttaaggatatccgcaaaaaggttgagcgccaggtttaccaaaaacttgagaagatgctgatcgacaaattgaactacttggttttcaaagatagagaagcctacgaaccgggaggtgttctcaacgcgtatcagctggcggctccgttcacttccttcaaagacatggggaaacagacgggtttcattttctacgtgcccgccgctaacacatcgaagatagactacgcaacgggttttatgaactttatatatcctaaaatcgaggatgcggtcgaattctttcaaaaattcgaatatattaggttcaaccccgacaaaaactactttgagtttgccgcccggtacaataattttgttaaggatgacgaagcaaagctccccgatgaatataatgaaccctggattatctgcactcatggagagcaacgcttccaataccagccttcttataaatcatttaaggaagtgaacgtgactcaggagctgcaagaccttttttccaaacacgccattatgccctacgcgcatggtaacgacctttcgccgcaaataaaagacaaagggaacacaagtttctacaaaggacttatcgcattgttgaggctggtcctccagatgcggtataccgacgggaagggccgcgactttatactttctccggttgctaatgatgagggggttttcttcaatagcgagaaggctaaggagacggagcccaatgatgcagacgcaaacggagcctatcacatagctcttaagggattgcttttgttgaaacggatcaaaggagagttcaagaaagaggaaaaaaccggaaaaaagaaaactcagaaagaaaattggatagatcttagtatatcgaataaagaatggtatgagttcgcacagaagaaggaatatcggaaa

SEQ ID NO: 16 - DNA, 4431 bp, Artificial Sequence
  synthetic
atgtcgaacttttttaaaaactttaccaatctctatgagctctctaagactctccgcttcgagcttaagccagtgggtgacacgctcaccaatatgaaagaccatctggaatacgatgaaaaactgcagacctttctcaaagaccagaacattgacgatgcttaccaggctcttaaacctcaatttgatgaaatacatgaagagttcataacagattctctggagagtaaaaaggcgaaagagatagatttttcagaatacctcgatttgtttcaggagaagaaagaacttaacgacagtgagaaaaagctcagaaataaaattggcgaaacgttcaacaaagctggtgagaagtggaagaaagaaaagtacccccagtatgagtggaaaaaggggagtaaaatagccaatggggcagacattctcagttgccaggatatgcttcagtttataaaatataaaaacccagaggatgagaaaatcaaaaattatattgacgatacgttgaaaggctttttcacatatttcggtgggtttaaccaaaatagagctaattattatgagacaaagaaagaagcaagtactgcagtggcgacgaggatcgtccatgaaaatttgccgaagttctgtgacaacgtgatacaattcaaacacataattaagaggaagaaggatggcactgtcgagaaaacagaacggaaaacagaatatctcaatgcctatcagtacctgaagaataataacaagataacacaaataaaggatgctgagacagagaaaatgatagagagcacgcccattgcggagaagatttttgatgtctactacttctcctcttgcctctcgcagaaacagattgaagagtataataggattatcggtcactataatttgcttatcaacctgtataatcaggcaaagcgctccgaagggaaacacttgtcagccaatgaaaaaaagtataaggatttgcccaaattcaaaacactgtacaagcagatcggttgtggcaaaaagaaagacttgttctacacaattaagtgcgacaccgaggaggaggctaacaagtcaagaaatgaagggaaggaatcacactccgtcgaagaaataataaacaaggcccaagaggctataaacaagtacttcaaaagcaacaatgactgtgaaaatatcaacacggtcccagacttcataaattacatccttaccaaggagaattatgaaggcgtctactggtctaaagccgccatgaacaccatttcggataagtactttgcgaattatcatgacttgcaagaccgcctcaaagaggcgaaagtctttcagaaagcggataagaaaagtgaggacgatataaaaatccctgaagcgatcgaattgtctggcttgtttggagttttggactcgctggcagactggcaaacaacattgtttaaatcatctattctgtcgaatgaagataaacttaaaattatcactgactcccaaacaccgtcggaagcgcttctcaaaatgatatttaacgatatcgaaaagaatatggagtcgtttctgaaggaaactaacgatatcatcactctgaagaagtataagggtaacaaagagggtacagagaagataaagcaatggtttgattacacgttggctatcaaccgcatgctcaagtactttctcgttaaagagaacaaaatcaagggaaactcgttggatacaaacatatccgaggccctcaagaccttgatctattcagacgacgcagaatggtttaaatggtacgacgcacttcgcaactatctgacacaaaaaccccaggatgaagccaaggaaaacaagttgaaattgaactttgataatccctccttggccggcggttgggacgttaacaaagagtgttctaatttttgcgtcattctgaaggataaaaatgagaaaaaataccttgctattatgaagaagggtgagaacacgcttttccagaaagaatggacagaaggccggggcaaaaatttgaccaagaagtccaacccactcttcgaaattaacaactgtgaaatactgtcaaagatggaatatgatttttgggctgatgtgtctaaaatgatccccaaatgctctactcagttgaaggcagtcgtgaaccatttcaaacagtcagataatgagtttatcttcccaatagggtataaggtcacctcgggtgaaaaatttcgggaggagtgcaagataagcaaacaggacttcgagctcaacaataaagtcttcaacaagaatgagctctcggtcacagcgatgagatatgacctgtcctcgacccaagagaaacagtacattaaggcctttcagaaggaatactgggagctcttgtttaagcaagagaagagagatactaagcttactaacaatgagatcttcaacgaatggataaatttttgtaacaagaagtacagcgaattgttgtcatgggaacggaaatataaagatgcgttgacaaactggattaatttctgcaagtatttcctctcgaagtacccgaaaacgactctcttcaattactcttttaaggaaagcgaaaactataacagcctggacgagttctatagagatgtggacatctgctcatataaactgaatattaacaccactattaacaagtccatactggacagattggtcgaagagggcaaactgtacttgtttgaaataaagaaccaagacagcaatgatggaaagtcaatcggccacaagaataaccttcacactatttattggaacgcaattttcgagaacttcgacaatagaccaaagctgaacggcgaagcagaaatattctataggaaagctattagtaaggacaaacttggtattgttaaaggcaagaagaccaaaaatggcaccgaaatcataaagaactatcgcttttctaaagaaaagtttatactccatgtcccaatcacattgaacttctgttctaacaacgagtatgtcaacgacatcgtcaacacaaaattttataattttagtaacttgcattttttgggcatagacagaggcgaaaaacacctggcctattattcactggtgaataaaaatggagagattgtcgatcagggtacgctgaacttgccgttcacagataaggatggcaatcaacgctcaattaagaaagagaaatatttctacaataagcaggaggataagtgggaagccaaggaagtcgactgctggaactataatgatctcctggacgcgatggcttccaatcgcgacatggcccgcaagaactggcaacggatcgggacgataaaggaagcgaaaaatgggtatgtttcacttgtcatccgcaaaatagcagatcttgcagtcaataacgaacgccccgcctttatagtcttggaagacctcaacaccggctttaagagatcgagacagaaaatagataaaagcgtgtatcaaaagtttgaactcgcgttggcaaagaaacttaacttcctcgttgataagaatgcaaaaagggatgagattggctctccaacgaaggccttgcaacttactccccctgtgaataactatggggacattgagaacaagaaacaggccggcattatgctctacacgagagctaattacacgtcccagactgaccccgctactgggtggcgcaagacgatataccttaaggcgggcccagaagaaaccacatacaaaaaagatgggaagataaagaacaagtctgttaaagaccaaattattgagacatttactgatatcgggttcgatgggaaagattattactttgaatatgataagggcgagtttgtggatgaaaaaacaggggagattaaacccaaaaaatggaggctttacagcggagagaatgggaaatctcttgatagattcagaggcgaacgcgagaaagataagtacgaatggaaaatcgataagattgatatagttaagattctggacgacttgtttgtcaacttcgacaagaacatttcgctgctgaagcaactcaaggagggcgtcgagcttactcgcaataatgagcatggaactggagaatcattgcgcttcgctatcaatctcatacaacaaatcaggaacactgggaataatgaacgggataatgattttatcctttcgccggttagggatgagaatggtaaacattttgattcacgcgaatattgggataaggaaacgaagggtgagaaaatctccatgccgtcaagtggggacgccaacggagcgtttaatatcgcgcggaaagggattatcatgaatgcccatattttggcaaacagtgacagcaaagatctttccttgtttgtctcagatgaagaatgggatcttcatctcaataacaagactgaatggaaaaaacagttgaatatattctcatcgcgcaaagcgatggccaagcgcaagaaa

SEQ ID NO: 17 - DNA, 3867 bp, Artificial Sequence
  synthetic
atgaagaaagaatcatttgagcggttcgtcggcgtctatccgatgtcaaagactctcaaaaacaggcttattccagtggggcggacagaggagtttatccggcaacgcgacattatttcccaagatgaacaaagagcgcgggattacgagtttattaaagaacggattgactctttccacgccgaactgatcgaggatgtgctcggagatcagaataactgccttgactggataggactcgcggaagcgattgcggctcataaacagccgaaatccgagaacaaagtggaatcggcggcgcgcgttaccgcggaacaagccacgatgaggaaggctataattaatcttttcttggcacggcctgattttaagctgctctttaagaaggagcttatatcagagatacttccaaattatttccaagacgacactgaggtggtggaagtgctgtcgaaatttaaaggattcactacttattttaccggattctttgaaaacagaaaaaatgtgtactcagcggatgaactctctacctccattgcacacaggatagttcatgttaattttggaagatacctcgaaaacttgtccgttctcagacgcatagagcaagagtacccaagcgcacttaaaaatgctgaagatgaactgaagactatattcccggatttcgagtttaaggatctcttcatccttcaaaacttcaacaaggtcttgtctcaaattggcatagatcgctataacgaacttattggaggtaggacactcgaaggcaataagaaagttcgcggaattaacgaacacgtcaatttggcccaacagcaggataaggcagatggcaaaaagcaaaaacttaaacttcagttgctccacaaacaaattcttagtgacaggaatacccgcagcttcatttatgaccccattgagaatgattcggaagtctactcggccgtccgcgttatgtctgatagcctcaatatgccgaatgcggactttgggggggcatcgagtttcgaaagggctctggagctgctccaacacatcagcaactatgatctctcacacatatttgttccggcaaaaaatcttgaaaagctctcttcccaatttcttggacaatggagcgtctccggggacgcgcttgacagatgtgctgtcgaaaaactcggcccggcctccggcatgactaaacggcaggccgagagctggcggaagagcggctgcttctcgcttagcgatatatgctcggccttggagacgtctggacaaacgcttgatctttcgctggcgggtattcgcttgggcgaccttaaggcgaaggctgaggaggcgtacgttaaggtgcttcaattggctgaggaatgtgttagcggtcagagaaaactgttggaggatgagaacgctacggataccttgcgggaatatttggacgcgctcttggagttcctccatgtcctgaagctgttcgaggcccctagtgaagaagaccgggaccttgacttttatgtcgattttgattacattgttgctgtcatgggtgacatagtcccattgtataaccgggttaggaactatgtcactcaaaagccttactcccaagaaaagttcaaactcaactttgagtcgcccacgttgtgcgcaggatgggacataaataaagagtcagataacgtggccacattgctcaagaaagacgggttgttctacctggcaatcatgaacccggaacatagaaagtctttcatcgacgctcctgaagcggctaccgaggatatctaccaaaaaatgatttataagctgctgcccggaccaaataaaatgcttccgaaagttttcttttgtgcatcaaacatagacttttacgacccctcggacaagataatgcggatatatgagcaaggtacctttaaaaaaggcgatacgtttagcctgaaggattgccataccttgatcgattttttcaaggactccatagatcgccacccggactggtcccaatttggttttaagttttctgagacatccagctacaaggacatagccggcttttatacagaagtccagcaacaaggatataaactccggttcgttgatgtcgatgcctcatacgttgacaaactcgtggaggagggtaaagtctacctctttaaaatttacaacaaggacttctcacttcatgttaggaatcgggagaagaagtcgagtaaaaacatgcatacgttgtacctggagagtttgttctctgtgcaaaatctggaagacgtggttttcaaactcaacggtgaagctgagttgttttatagacgcggatcgataaaaaaaccagtgatacataaagcaggagagaagttggttggtcggacttatcaagagattgacaagacgaccggagaaacgatcaccaggcacatgccggaaaaagtgtatcttgagctgtttaagaatcttaacggtaaattggcctataagagcctgtgcgataaggcaaaggaactcctcgacaataagaaggttgaatcccgcgtggccgaatatgatattgtgaaagacaaaagatacacgaaagaccagtatttctttcacgtgccaattaccatcaactttaaagctacagggtcccagccttttaatagcaacgcgcttgagtatttggatggtaatcgggaggtgaaaatcataggaatagaccgcggagaaagacatctcctgtactatacccttatggatcagcaaggcaacataattgatcaaaactcactcaactcactcaatggtgtggattacagggaaaaactccacgaacgggaagaggagaggaaagaggcccggaagtcttggaaaagaatcagcaagattgccgatcttaaggaaggttatctgtctctcgcggtgaacaaacttgctaagatgatgattgacaataatgccatattggtgctcgaggatctcaacttcgggtttaagcaggggcgggtcaaaatcgagaagcaagtctaccaaaagttcgaaaagcaactcattgacaagctgaactattttgcgttgaaacccaacgacgttcctatcaatcagccggggggagtgttgaaagggtatcaactggccgcgccatttgaaagctttgaaaagcttggcaaacaaagcggctttatcttttacgtcccggcttggaacaccagccatattgaccctactacgggcttctgcgacctctttctgtggaacagagagagtactgacgcggcgggtttcttcagtaggatggagacaatcagatataacgctgagtcagattatttcgaattttcgtttgactacgccgattaccacacgaggtgtttgtcgccgcaaactaagtggacagtttgttcccacggggacagacgctggatctggaccgcgcggactacaaagaccggtcgggattatgaggttgatgagatcaatgtcaatcaagagcttaaggatatatttgacagtgccgggattaattatcacgacggccacgatttgaggggtgacattgctgcacaggagactaaggagttcggaaagaaaatgcttaggcaatttaaaaatctccttaagctgaggtattctacagcgcaatcggatgaggaacaacacgactgcattatttcacccgtgatgaatgataacggggaattttttatgtccaaagaaggatgcaagaacttgcccgagaatgcggacgccaatggcgcatacaatatcgcgctgaagggccttcagcttttgagggagcaaattgaagttaatgaaaaaacagggctccggcggattgcctaccccaaaaaacccaacgaggcgtggctgcggtttgtgcaaggcaaagaatggatggaa

SEQ ID NO: 18 - protein, 1295 aa, Brumimicrobium aurantiacum
MKNQINLFTNKFQLSKTLRFELKPQGKTLEHINSKGFIKNDEKRADSYKKMKATIDAFHRDFIDLAMSNVKLTNLIDFEEIYNASNADKKDEKYKTKLSKIQEILRKEIAKGFKGEEVKDIFSKIDKKDLITKLLEEWIIENKIEDIHFDPEFKNFTTYFSGFHQNRKNMYTDQEQSTAIAYRLIHENLPRFIDNINIFQKINKVPDLEENLKKLYQEIEEYLGINAINEAFELEYFNETLSQKGIDIYNLILGGRTAEEGKQKIQGLNEYINLYNQKQDKKNRVPKLKVLYKQILSDRTRTSFLPDTFEDDEESSASQKVLDSINNFYLENLIDYLPNDKNSTINVLENLKLLLAELINFELDKVYIKNDTSITNISMKIFKNYSVIREALNYFYENKIDPNFAHNFNNANTDKKREKLEKEKAKITKQTYLSISFIEEAIHLYINENSNGNQYKNTYKPNCIANYFKDFFIAENKEGSNKEFDFISKIKARYNTIKGVLNTPFPDNKRLHQEKNNIDNIKHFLDSIMEYLHFAKPLVLSGSFAFEKDEQFYTNFDELYNQLELIIPLYNKVRNYATQKPYSTEKFKLNFENSTLLNGWDVNKEEANTSILFIKNGFYYLGIMDKNHNKIFRNTPKSTNTDIYKKVNYKLLPGASKMLPKVFFGKKNLDYYKPSKDILRIRNHGTHTKGGKPQSGFDKLDFNLNDCHKLIDFFKDSIQKHPDWSKFKFKFSDTQIYESIDQFYRELEPQAYSITYTNIDSSFIEEQINEGKLYLFQIYNKDFSKFSNGKPNLHTLYWKALFDEQNLKDVTYKLNGEAEIFYRKKSIQHDRQIIHKRNQPIINKNPNNEKKESIFKYNIIKDKRYTIDKFQFHVPITLNFKAKGTDYINYDVLDYLKENPDVKIIGLDRGERHLIYLTLIDQKGKILEQISLNEIVNKKHNITTSYHNLLETKEIERDKARKNWGTVETIKELKEGYISQVVHKISKMMIEHNAIVVMEDLNMGFKRGRFKVEKQVYQKLEKMLIDKLNYLVLKDRQPNEPAGIYNALQLTNKFESFQKLGKQSGFLFYVPAWNTSKIDPTTGFVNLFHVKYESVRKSQEFFNKFNSIKYNPKEAIFEFDFDYNEFTTRAEGTKTNWTVCTYGDRIKTFRNPEKLNQWDNKEINITTAFEDFFGRHNITYGNGSDIKSQLISREEKDFFSELIHLFRLTLQMRNSKTNSEIDYLISPVKNENGFFYDSRHADKNLPKDADANGAYHIAKKGLQWIKEIQSFEGNEWKKLKLDKTNKGWLKFVQEN

SEQ ID NO: 19 - protein, 1463 aa, Saccharobesus litoralis
MSDRLDVLTNQYPLSKTLRFELKPVGATADWIRKHNVIRYHNGKLVGKDAIRFQNYKYLKKMLDEMHRLFLQQALVLEPNSNQAQELTALLRAIENNYCNNNDLLAGDYPSLSTDKTIKISNGLSKLTTDLFDKKFEDWAYQYKEDMPNFWRQDIAELEQKLQVSANAKDQKFYKGIIKKLKNKIQKSELKAETHKGLYSPTESLQLLEWLVRRGDIKLTYLEIGKENEKLNELVPLVELKDIHRNFNNFATYLSGFSKNRENVYSTKFDRRSGYKATSVIARTFEQNLMFCLGNIAKWHKVTEFINQANNYELLQEHGIDWNKQIAALEHKLDVCLAEFFALNNFSQTLAQQGIEKYNQVLAGIAEIAGQPKTQGLNELINLARQKLSAKRSQLPTLQLLYKQILSKGDKPFIDDFKSDQELIAELNEFVSSQIHGEHGAIKLINHELESFINEARAAQQQIYVPKDKLTELSLLLTGSWQAINQWRYKLFDQKQLDKQQKQYSFSLAQVERWLATEVEQQNFYQTEKERQQHKDTQPANVTTSSDGHSILTAFEQQVQTLLTNICVAAEKYRQLSDNLTAIDKQRESESSKGFEQIAVIKTLLDACNELNHFLARFTVNKKDKLPEDRAEFWYEKLQAYIDAFPIYELYNKVRNYLSKKPFSTEKVKINFDNSHFLSGWTADYERHSALLFKFNENYLLGVVNENLSSEEEEKLKLVGGEEHAKRFIYDFQKIDNSNPPRVFIRSKGSSFAPAVEKYQLPIGDIIDIYDQGKFKTEHKKKNEAEFKDSLVRLIDYFKLGFSRHDSYKHYPFKWKASHQYSDIAEFYAHTASFCYTLKEENINFNVLRELSSAGKVYLFEIYNKDFSKNKRGQGRDNLHTSYWKLLFSAENLKDVVLKLNGQAEIFYRPASLAETKAYTHKKGEVLKHKAYSKVWEALDSPIGTRLSWDDALKIPSITEKTNHNNQRVVQYNGQEIGRKAEFAIIKNRRYSVDKFLFHCPITLNFKANGQDNINARVNQFLANNKKINIIGIDRGEKHLLYISVINQQGEVLHQESFNTITNSYQTANGEKRQVVTDYHQKLDMSEDKRDKARKSWSTIENIKELKAGYLSHVVHRLAQLIIEFNAIVALEDLNHGFKRGRFKIEKQVYQKFEKALIDKLSYLAFKDRTSCLETGHYLNAFQLTSKFKGFNNLGKQSGILFYVNADYTSTTDPLTGYIKNVYKTYSSVKDSTEFWQRFNSIRYIASENRFEFSYDLADLKQKSLESKTKQTPLAKTQWTVSSHVTRSYYNQQTKQHELFEVTARIQQLLSKAEISYQHQNDLIPALASCQSKALHKELIWLFNSILTMRVTDSSKPSATSENDFILSPVAPYFDSRNLNKQLPENGDANGAYNIARKGIMLLERIGDFVPEGNKKYPDLLIRNNDWQNFVQRPEMVNKQKKKLVKLKTEYSNGSLFNDLAFK

SEQ ID NO: 20 - protein, 1260 aa, Unknown
  Clostridiales bacterium RUG149
MKKIDSFTNCYSLSKTLRFKLIPIGATQSNFDLNKMLDEDKKRAENYSKAKSIIDKYHRFFIEKVLSSVTENKAFDSFLEDVRAYAELYYRSNKDDSDKASMKTLESKMRKFIALALQSDEGFKDLFGQNLIKKTLPEFLESDADKEIIAEFDGFSTYFTGFFNNRKNMYSADDQSTAISHRCINDNLPKFLDNVRTFKNSDVANILNNNLKILNEDFDGIYGTSAEDVFNVDYFPFVLSQKGIEAYNSILGGYTNSDGSKIKGLNEYIYLYNQKNGNIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSVESFSLADIQKYADVSESSEANAEVTEWLRNEIKEKCNLAVQGYESSKDLISKPYTESKKLFNNDNAVELIKNALDSVKELENVLRLLLGTGKEESKDEYFYGEFLPCYERICEVDSLYDKVRNYMTQKLYKTDKIKINFSNSHFLSGWAQTYSTKGALIVKKENNYYLVIVDKKLSNDDIVFLGTNTQLSPAERIVYDFQKPDNKNTPRLFIRSKGTSYAPAVKEYDLPISDIIEIYDNEYFKTEYRKINPKGYKEALIKLIDYFKLGFSRHESYRCFNFKWKESEQYSDISEFYNDVVKSCYQLKSESINFDSLLKLVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNFGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 21 - protein, 1368 aa, Unknown
  Candidatus Campbellbacteria bacterium RIFCSPLOWO2_01_FULL_34_15
MKKEKEFKSFGDFTNLYEISKTLRFELKPVENTQTMLDEADVFGKDKVIKDKYTKTKPFIDKLHREFVDESLKDVSLSGLKKYSEVLENWKKNKKDKDIVKELKKEEERLRKEVVEFFDNTAKKWANEKYKELGLKKKDIGILFEESVFDLLKEKYGEEQDSFLKEEKGDFLKNEKGEKVSIFDEWKGFVGYFTKFQETRKNFYKNDGTETALATRIIDQNLKRFCDNIDDFKKIKNKIDFSEVEKNFNKTADVFSLDFYNQCLLQKGIDSYNEFIGGKTLENGKKLKGVNELVNEYRQKNKNEKVSFLKLLDKQILSEKEKLSFGIENDEQLLVVLNSFYETAEEKTKILRTLFGDFVEHNENYDLDKTYISKVAFNTISHKWTNETHKFEELLYGAMKEDKPIGLNYDKKEDSYKFPDFIALGYLKKCLNNLDCDTKFWKEKYYENNADKKDKDKGFLTGGQNAWDQFLQIFIFEFNQLFNSEAFDNKGKEIKIGYDNFRKDFEEIINQKDFKNDENLKIAIKNFADSVLWIYQMAKYFAIEKKRGWDDDFELSEFYTNPSNGYSLFYDRAYEEIVQKYNDLRNYLTKKPYKEDKWKLNFENPTLANGFDKNKESDNSTVILRKKRKYYLGLMKKGNNKIFEDRNKAEFIRNIESGAYEKMAYKYLPDVAKMIPKCSTQLNEAKNHFRNSADDLEIKKSFSNPLKITKRIFDLNNIQYDKTNVSKKISGDNKGIKIFQKEYYKISGDFDVYKSALNDWIDFCKDFLSKYDSTKDFDFSILRKTKDYKSLDEFYVDVAKITYKISFTPVSESYIDQKNKNGELYLFEIYNQDFAKGKMGAKNLHTLYFENVFSPENISKNFPIKLNGNAELFFRPKSIESKKEKRNFVREIVNKKRYSEDKIFFHCPITLNRETGSIYRFNNYVNNFLSENNINIIGVDRGEKHLAYYSVIDKNGVKIGGGSFNEINKVDYAKKLEERAGEREQSRKDWQVVEGIKDLKKGYISQVVRELADLAIKHNAIIVLEDLNMRFKQIRGGIEKSIYQQLEKALIDKLSFLVEKGEKDPNQAGHILKAYQLAAPFTSFKDMGKQTGIVFYTQASYTSKTCPNCGFRKNNNKFYFENNIGKAQDALKKLKTFEYDSENKCFGLSYCLSDFANKEEVEKNKNKKRNNAPYSDIEKKDCFELSTKDAVRYRWHDKNTERGKTFFEGESVYEEKEEKEIGQTKRGLVKEYDISKCLIGLFEKTGLDYKQNLLDKINSGKFDGTFYKNLFNYLNLLFEIRNSISGTEIDYISCPECQFHTDKSKTIKNGDDNGSYNIARKGMIILDKIKQFKKENGSLDKMGWGELFIDLEEWDKFAQKKNNNIIDK

SEQ ID NO: 22 - protein, 1238 aa, Unknown
  Candidatus Uhrbacteria bacterium CG11_big_fil_rev_8_21_14_0_20_41_9
MKDFYQFTNLYALSKTLRFSLIPTPATKQMLEDAKVFEKDETIQKKYEATKPYFDRLHREFALEALQDQKLDFKNYLELYRKYKADKKASGKLLINIEKDLRKEVVKLFDKQGEKWAKQYPGLKNKNIGVLFKEAVFTVILKERYGNEKETQILDESSGQLVSIFDSWKGFIGYFKKFHETRKNFYKDDGTSTALATRIIDQNLKRFCDNILIFESTKEKVDFSEVEISFGKPLSEVFTLEFYNTCFLQNGIDFYTKILGGETLQNGEKVKGLNECINLHKQKTGEKLPFFKSLDKQILSEKDKFFIDEISNETQLLEVLKSFVASAESKTDTIKTLVDDFVKDQDKYDLNYIYFSNDGLNTITRKWTTETQVFEEALYTALKAAKVVSSSAKKNEGGYSFPDFIPFAHLKTALESIKIDGTIWRDNFNAIENFEEKSIWAQFLAIYNFELSNLFETEIKNPEIGNCPTIGYNVYKQDFEELLKSFVYDPNAKVTIKNFADNVLSIYQMAKYFAVEKKRGWNTDYELDVFYTDPQNGYLQYYENAYEEIVQVYNKLRNYLTKKPYSEEKWKLNFDSGTPIKYTTRAIIFNNTTNERYYLGLLKKGVAKPREFEPINNNIISSGEFRRMIIQQLKFQTLAGKGYVRDFGVKYSEDKDGVKHLQQLIKKQYLSKYPCLKKIADGVYNDKKAFDADIKDVLLETYNLDFQPISEEFILNKNRLGEIYLFEIHNKDWNLKDGKNKSGSKNLHTMYFESLFVDKTTFKLNNEGAEVFYRPATNEGKLGTKKDRNGKIIINHKRYATDKILFHCPIGLNKDAGKSYTFNAKINNMLANNPDINIIGVDRGEKHLAYYSVITQKGKILDRGSLNKVEGGDKQEIDYAKKLEETAKNREQARKDWQAVEGIKDLKRGYISQVVRKLADLAIEHNAIIVFEDLNMRFKQIRGGIEKSVYQQLEKALIDKLSFLVMKGEADPEKAGHLLKAYQLVAPFESFQSMGKQTGIIFYTQANYTSKIDPITGWRPNLYLKYTSAEKAKADILKFSKIEFVNNRFELTYDIKNFVLDKKVVLSNKTKWTVCSSVERFRWNRRLESNQGNYEHYENLTENLSSLFKDFGFEIEQNIIRQVEQLATKGNEQFFRSFIFYVNLIFQIRNTDAKAKDQNKEDFILSPVEPFFDSRTPEKFGENLPENGDDNGAFNIARKGIIMLNKISAYKQEVGNVDKIIWKDLFISAAEWDNFTQE

SEQ ID NO: 23 - protein, 1234 aa, Eubacterium ventriosum
MESNNKIFTETIGTSSIAKTMRNSLVPTESTKRNIEKNGIIIDDQLRAEKRQQLKEIMDEYYRTYIDNKLSNVALTRTIDWKELFQAIEDNYKQNTTKTKNELEKKQKEKRTEIYKILSDDEKFKQLFNAKLLTNVLPEFIKNQNIDNEEKQEKISTVELFQRFTSSFTDFFKNRKNVFSKDEISTSICYRVVQENAWIFYQNLLAFEEIEIFDFDFYGLLLNQGGIRFYNDVCGKINYHMNLYGQKHNIKSNKFKMKRMHKQILSIDESTFEVPTMFENDKEVYQVLNEFLSDLASKKILERVEKIGENVSEYEINKIYIQSKNFEKFSSFMCGNWQIINDSLKTYYNEKIKSKGKAKEEKVKKAIKAIEYKSLADINQLVERYNNDELNRKAEEYISAINEKIKDLDVNEIEYDEKINLIENETKSEEIKSKLDSIMEIMHWTKMFIIEEEIEKDVNFYNEIEEIYDELQPLVTIYNRIRNYVTQKPYSEEKIKLNFGIPTLANGWSKTKEYDNNAIIMIRDGKYYLGIFNAKNKPDKKIMEGHQSEENGDYKKMIYRLLPGPNKMLPKVFMSKTGIAEYKPSQYILECYEQNKHIKSDKNFDIKFCRDLIDFFKTSINRHPEWSKFNFKFSETSEYEDISTFYREVEKQGYKIEWTYISEKEIKELDENGQLYLFQIYNKDFSEKSKGKENLHTMYLKNLFSEENLKNIVLKLNGEAEVFFRKSSIKKPIIHKKGSVLVNKTYNENGERKSIPEEQYTEIYKYLNSIGTNELSEKSKKLMEEGKVEYYKANYDIVKDYRYSVDKFFIHLPMTINFKAAGFSPINNIALKNIALKDDMHIIGIDRGERNLIYVSVIDTKGNIVEQRNFNIVNGIDYKEKLKQKELDRDNARKNWKEIGKIKDLKEGYLSLVVHEIAKLVVKYNAIITMEDLNQGFKRGRFKVERQVYQKFETMLINKLNYLVDKDLAVDQEGGLLRGYQLTYIPESLKVLGRQCGYIFYVPAAYTSKIDPTTGFVAIFNYKGMTDKDFVTSFDSIKYDDERGLFAFEFDYENFVTHKVEMARNKWTVYTYGERIKRKFKNGSWDTAEKVDLTYQMRSILEKYEIEYNKGQDILEQIEELDEKAQNGICKEIKYLVKDIVQMRNSLPDNAAEDYDAIISPVINNNGEFFDSTRGDEDKPLDADANGAYCIALKGLYEVMQIKKNWNEETEFPRKELKIRHQDWFDFIQNKRYL

SEQ ID NO: 24 - protein, 1298 aa, Unknown
  Hydrogenovibrio sp. XS5
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 25 - protein, 1323 aa, Unknown
  Parcubacteria bacterium JGI MDM2 000213CP-K14
MTNTKQNSVFQDFTRKYALSKTLRFELRPVGNTREMLDAAGVFEKDRIIKEKYERTKPYFDRLHREFVQESLSDVALTGVAEYLSIFSEWRTDKKDKTKAKQLSDIEKSLRNEVTRFFEETAKIWAEKYSSIKIKKSGIDFLFEEGIFRVLKERYGNEDDAFVEDDAGNRISIFDQWKGFVGYFTKFYETRKNFYKDDGKASRIATRIVDENLKRFCDNIIAFDRLKQVLDLSEVQGHLKDSLTEVFSPRFYDECLLRQGIDRYNTILGGDSMDQNVKGINQYVNKYLQDHKGERLPFLKPLDKQILSEKESFLEGIESDGELLSVLELFQKNAGKKVGIFRSLMEKFVSKNDAFDLSKIYLSKEGFNTISRKWTDNTRDLESSLHKAMKADKPTGLSYDKTEDLYKFPDHIALSYLQEAISAQNQENTVWKERYYKTEENSSGVLFGNESVWEQFLHILSFEISSLYSTETTHTIAEKEDFATALINAERFEEVFSSDSIVRTIQTKVGYDVFEQKMTSVSGPEFGPDEKVFIKNYADSVLRIYQMAKYFSLEKKGVWNEEYELDSFYSEPEHGYLVFHENVREEIVKVYDALRNYLTKKPYSEEKWKLNFENPTLADGWDKNKESDNSAVILRKDGQYYLGVMKKGKTHIFSERNEKEMGIGGGGMSYEKMVYKLFPDPAKMMPKVCFSAKGLEFFNPPADIMEIYEKGTFKKGENFDIGSMRRLIDFYKECLALYPGWKGYDFSNVKESATYVTNIGEFYSDVAKAGYKVEFVAISESYVGEKNEAGELYLFEIHNKDWNLKDSKRKDGAKNLHTLYFESLFSEENVEADFPMKLNGQAELFYRPATATDKLGFKKNADGTDMVGKNGQKVVNHKRYSEDKILFHVPMTMNRGAGNIYRFNDEINELLAGNPAINIIGVDRGEKHLAYYSVINQKGEVLESGSLNTVNGIDYAGKLDERAKGREQARKDWQAVEGIKDLKKGYVSQVVRKLADLAIEYNAIIVLEDLNMRFKQIRGGIEKSAYQQLEKALIDKLSFLVEKGEKDSESAGHLLRAYQLAAPFESFQEIGKQTGIIFYTQASYTSKIDPATGWRPNLYFKYTSAKKAKEDIERFDAIHFRDGYFEFSYDLGKIVNSEKGATIFPERTDWTVGSCVERYRWNRKLNAGKGGYDYYSDLTEDFRSLFTSVGIGEEGDIKAQIAALDSEGNERFFRDFIFFFSLICQIRNTDPDAKDPDKADFILSPVAPFFDSRTPEAFGTKLPKNGDDNGAYNIARKGIIILDKISDYKRANGSCKNLGWGGLYISHAEWDDFARKNALKNSG

SEQ ID NO: 26 - protein, 1260 aa, Porphyromonas cansulci
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 27 - protein, 1310 aa, Sedimentisphaera cyanobacteriorum
MKEFTNQYSLTKTLRFELRPVGETAEKIEDFKSGGLKQTVEKDRERTEAYKQLKEVIDSYHRDFIEQAFARQQTLSEEDFKQTYQLYKEAQKEKDGETLTKQYEHLRKKIAAMFSKATKEWAVMGENNELIGKNKESKLYQWLEKNYRAGRIEKEEFDHNAGLIEYFEKFSTYFVGFDKNRANMYSKEAKATAISFRTINENMVKHFDNCQRLEKIKSKYPDLAEELKDFEEFFKPSYFINCMNQSGIDYYNISAIGGKDEKDQKANMKINLFTQKNHLKGSDKPPFFAKLYKQILSDREKSVVIDEFEKDSELTEALKNVFSKDGLINEEFFTKLKSALENFMLPEYQGQLYIRNAFLTKISANIWGSGSWGIIKDAVTQAAENNFTRKSDKEKYAKKDFYSIAELQQAIDEYIPTLENGVQNASLIEYFRKMNYKPRGSEEDAGLIEEINNNLRQAGIVLNQAELGSGKQREENIEKIKNLLDSVLNLERFLKPLYLEKEKMRPKAANLNKDFCESFDPLYEKLKTFFKLYNKVRNYATKKPYSKDKFKINFDTATLLYGWSLDKETANLSVIFRKREKFYLGIINRYNSQIFNYKIAGSESEKGLERKRSLQQKVLAEEGEDYFEKMVYHLLLGASKTIPKCSTQLKEVKAHFQKSSEDYIIQSKSFAKSLTLTKEIFDLNNLRYNTETGEISSELSDTYPKKFQKGYLTQTGDVSGYKTALHKWIDFCKEFLRCYRNTEIFTFHFKDTKEYESLDEFLKEVDSSGYEISFDKIKASYINEKVNAGELYLFEIYNKDFSEYSKGKPNLHTIYWKSLFETQNLLDKTAKLNGKAEIFFRPRSIKHNDKIIHRAGETLKNKNPLNEKPSSRFDYDITKDRRFTKDKFFLHCPITLNFKQDKPVRFNEQVNLYLKDNPDVNIIGIDRGERHLLYYTLINQNGEILQQGSLNRIGEEESRPTDYHRLLDEREKQRQQARETWKAVEGIKDLKAGYLSRVVHKLAGLMVQNNAIVVLEDLNKGFKRGRFAVEKQVYQNFEKALIQKLNYLVFKEVNSKDAPGHYLKAYQLTAPFISFEKLGTQSGFLFYVRAWNTSKIDPATGFTDQIKPKYKNQKQAKDFMSSFDSVRYNRKENYFEFEADFEKLAQKPKGRTRWTICSYGQERYSYSPKERKFVKHNVTQNLAELFNSEGISFDSGQCFKDEILKVEDASFFKSIIFNLRLLLKLRHTCKNAEIERDFIISPVKGNNSSFFDSRIAEQENITSIPQNADANGAYNIALKGLMNLHNISKDGKAKLIKDEDWIEFVQKRKF

SEQ ID NO: 28 - protein, 1475 aa, Unknown
  Bdellovibrionales bacterium SP5OBV1
MIYNVKLKKLTNSSDVFSLFSRKYSLSKTLKFELKPTAETKQHLQDFIVSDTKRAKEYKELKKIIDEFHKDYIELTLSHKNILDKDKLTDFCKLWSDNNLLDEIKEKHNLKEKTKEEVIKKMEQQFRKDIVEQFKTLNPLLERFFKTADEEQLLGFLKKLDNKLWTKFKEKKEKALLDEYLQEPIKQKNIEKSILFSSELIKYLLPVWLKNSQLTDKEDKQKIIKNFGRFTTYLTGFNENRKNMYSVEEQGTAIAHRIINENLMKFLGNLQAYEKIKSSHPELQQSFKNMKSDFKEEFDYFSLENIEDLFKPEFFNSCLSQKEIDFYNTLLGGKTVEDGKKLQGINEYINLYRQKKKSEDIDIKIKYSNKNLPTMELLYKQILSDRESHSFILAEFENKKELLEALKSFWSVLFEEKTYQDYFHTKKKTSLWNNLHSLLTGRSYYDLEGAYFKSSELNKLSHNLFKDYRIITEALNENYDKIKVTLESYKKVLKKEDKKEFEKNIKSLKGFQTEWKKILSFEKNKEELKFSDEILASYFEFPIDKKTKKKNQKDFYSLQEIKDHIELYSKESDELKEDLDNLKKKLKDYKKDNIISSWFKYQFESKQNLDFFLQRNHEKENEDKREQNKNSLFSHIENHYQKIQNLSLDEKEFKKEEVEDIKFFLDLILHVLHLMKPFYLEGKASNLLDKGINNEIEVLYKKLKPIEKLYNQTRNYIAKKKSRYNKVKINFEDSTLLDGWDLNKEKDNLAVLLRKKDAVIGWKYYLGVMNKTTKDLFDYHIKLDDSEKVKQKKEELQDLILHRENDGNFYEKMNYKLLPDPSKMLPKVFFSKKNLDSFKPSEEIVRIRDNKTYAKNGGQDFSKADCHKFIDFYKESLKKHKEWNDFFKFNFSPTSQYNDISDFFQEVKNQGYNLQFDKIKSSYIEEKVKTGELFLFEIYSKDFSTKSKNRKNSKDNLHTIYFKGLFEKENLKDTVLKLNGKAEIFYRKATKKFNITHKKNTELENKNKNNPNKHSIFNYDLIKDKRFTEDKFFFHFPISLNFNSKGMKSYLFNQEVLKCLKGNKEVNIIGIDRGERHLAYYTIINQKREVLSQGSFNKMESSYKDNSGKEVKIEKDYHELLESKEKERDKSRKEWNKIENIKELKSGYLSYLVHKISKLMIEHKAIVIFEDLNLGFKRGRFKFEKQVYQKLEKALIDKLNYLVFKDKKSSELGGYLNAYQLTAPFESFQRMGKQTGYLFYVPAYYTSKVCPLTGFINLIYPKYKNVKESQQFFEKFDRIYFDKNKNYFVFEYQDKKVNPSKKTESIETLWKVCTHGEERYKWDVKDRKMIEVNVTENLKKLFEEHKIEYRQIADLKSTITKQEKKDFFSKLIDGLKITLQLRHINPDSKDEKEKDFILSPVADESGRFFDSRKAKEGEPKNADANGAYHIALKGLRTLENITSDKKDKLKLQAITNKDWFSFLKENSNKKIPKVG

SEQ ID NO: 29 - protein, 1061 aa, Unknown
  Candidate division WWE3 bacterium CG10_big_fil_rev_8_21_14_0_10_35_32
MNKADSVFSNFTNQYSLSKTLKFELKPHPLTKSLVEVVNKDKEIDRLSTKEMKPMLDALHEKFITESLEKMIFSISDLEQLERHLDDLKVLRQKLKNLNNNKDKKKKEIDSIQKEIKNLEDKKSGKIPKLQQKLRQVIVDQINEIGDEWKEIYTKEGIKFEKDNGNKKKQGYEMLTTKGVLGILKSEYSQYPEKIKAISEFDQFFTYFKGLNDNRANYYTDEQKATGVANRIINVNLNIFLKNKNDFSLFLEKLPQLKDYETSFALSNFQKCLAQADIEAYNEKVGEVKKLVNLEYNQKVADKKDLLKGLNKLQKQIGCKIKQEREQLERGESIYPKYLEKVGLGFHITKTSDDKYQIWESLDYLNDQLIPQVKKLKDNYESFFENWQDYKLNEIWFRRESLNTISGRWFGGNNWFVLTKALAYFGTGKMNKGEYKPDQFISLLELKEAMEALENGVDFDIKKPNKKTKEGQTEAIKDEITKQYFYKPENLFRDKYKDIYKNKTLFVAFLTIWRYELKHRFKEIEFYLDDFNIQKKTPFDKGNKEHVKTVFNLMQEGYLPILQMTKYHSLEKKSEIVPGYNTENKFYETLNDFWEDNPINQYRSAFEATLTQKPYSEDKIKLNFDCGSLLGGWSPTYEAYGSLIFKKDNRFYLVLIKGTGLSDEEMKGLYQSIDRKNSIERLIYNAQKLDFKNCPRWFINSIGDSKAPAVKKYNLPVESIWEDYQIYRKLDKNGKDKYLEDNKNFRFRLIDYYKHCLPFHESLKEFKGNFSWKDTKDYNNLNEFYTDAVNSCYKISWENINFDALKRLVRNERIYLFQIYNKDFEIDTEIGEKKYGESYNTKEKLRKAQGNPKGVVNLHTQLFFELLKPENIKYLKLMGGGEIFFREPSKEKSYRFDTGRREVLKAKRYYEEKYFLHFPVEIKKTKESSESFNQKIYKTLTQNHKPCKIIGIDRGEKHLLYYSVISINESGGTKIEDQGSLNRVKTKSPVDEKKLECEYDEKGKLQKVDLVSTGKKVNYVDYHLLLDYYEKKRNLARKSWEVIGKIKDLKEGYLSQVIYKI

SEQ ID NO: 30 - protein, 1313 aa, Unknown
  Candidatus Woesearchaeota archaeon CG10_big_fil_rev_8_21_14_0_10_37_12
MSKLHFATLLTGAIMGVVRIDDKLKKLFWDLYSLRKTLRFELKPQGKTLEHIENKGLISQDEQRAESYKKMKKTINGFHKHFIEIAMSQVKLTKLNEFADLYNASPERKKEDGFKKAFEKIQADLRKDIAKAFNVGEAKEIFSKIDKKELVTELLEKWIRTQEEDLYFDERFKTFTTYFGGFHENRKNMYSDKEQSTAIAYRLIHENLPKFLDNIKVFERIKEISELYKNFPALYKEIKEYLDIEQIDEAFELYYYNKILTQKQIDAYNLIIGGMTAQEGKKKIQGLNEYINLYNQKQDKKDRIPTLKQLYKQILSDKESVSFLPDEFEDDAEETASQKVLNAIEEYYQANLVSFHPSGKDEAENVLEKIKELLTSLKDYDLNKIYLRNDKAITDISKTLFGDWNIIKAALEFAFLQTLETGKKGLSKKQEKEKEKYLEQSYFSIAEIEKALFAYKNEAEVLKDLKENTTPIADYFHTHFLAKKKEESDKEFDMIANIQAKYSCIKGILNTDYPKENRLHQDKKTIGDIKAFLDSLMELLHFVKQISLPSDTPLEKDEIFYSQFQPWYDQLQLLIPLYNKVRNYASQKPYSIEKFKLNFQSSTLAQGWDLNKEHNKNYSLIFKENDSFFLGIINIDDDKDVLRKDKRPEIFVDDSPLKKMVYKDLGDIKRQLPRIAFSDKAKKDVEDIGWNQNIEAIKKEFDKFQDAKKNDKDLWKNEFDRNKLSELIDYYKGVLKNHSERYEDTYKLEYKSTESYKNLGEFFDHIASQTYKVTFVGIDKNYINQLVDQGKLYLFQIYNKDFSKYSKGKPNLHTLYWKALFDPENLKNVMYKLNGGAELFYRKKSIKDLTIHKSNEPIKNKNPNNPKKESFFEYDIVKDRRYSVDKFQFHVPITLNFKATEKKYINNTVLGFLKDNPDVNIIGIDRGERHLLYLTLIDQKGKILEQRSLNTIVNNRVNIKTDYHKLLHNREKERDKARKDWGTIETIKELKDGYISIIVHEISRMMVEHNAIVVMEDLNFGFKRGRFKVEKQIYQKFEKKLIDKLNYLVFKDKANNETGGLYKALQLTSKFESFTKLGKQSGFLFYVPAWNTSKIDPTTGFVNLFNTRYENVDKAKTFFNNFENIRYNTDKDNFEFEVKKYSLFNSKVDDTIQDWVICTHGERIKTFRNSEKNNQWDNKEIVLTEEFKNLFKEYNINYKRDLKNQINSQTNKDFFEQLLNLFKYTVQMRNSITNSETDYLISPVINSEGKFYDSRNAGDELPKDADANGAYHIAKKGLWVLQQINAHEGEDWKKLKLAISNKEWLQFVQARAK

SEQ ID NO: 31 - protein, 1285 aa, Unknown
  Candidatus Roizmanbacteria bacterium GW2011_GWA2_37_7
MKSFDSFTNLYSLSKTLKFEMRPVGNTQKMLDNAGVFEKDKLIQKKYGKTKPYFDRLHREFIEEALTGVELIGLDENFRTLVDWQKDKKNNVAMKAYENSLQRLRTEIGKIFNLKAEDWVKNKYPILGLKNKNTDILFEEAVFGILKARYGEEKDTFIEVEEIDKTGKSKINQISIFDSWKGFTGYFKKFFETRKNFYKNDGTSTAIATRIIDQNLKRFIDNLSIVESVRQKVDLAETEKSFSISLSQFFSIDFYNKCLLQDGIDYYNKIIGGETLKNGEKLIGLNELINQYRQNNKDQKIPFFKLLDKQILSEKILFLDEIKNDTELIEALSQFAKTAEEKTKIVKKLFADFVENNSKYDLAQIYISQEAFNTISNKWTSETETFAKYLFEAMKSGKLAKYEKKDNSYKFPDFIALSQMKSALLSISLEGHFWKEKYYKISKFQEKTNWEQFLAIFLYEFNSLFSDKINTKDGETKQVGYYLFAKDLHNLILSEQIDIPKDSKVTIKDFADSVLTIYQMAKYFAVEKKRAWLAEYELDSFYTQPDTGYLQFYDNAYEDIVQVYNKLRNYLTKKPYSEEKWKLNFENSTLANGWDKNKESDNSAVILQKGGKYYLGLITKGHNKIFDDRFQEKFIVGIEGGKYEKIVYKFFPDQAKMFPKVCFSAKGLEFFRPSEEILRIYNNAEFKKGETYSIDSMQKLIDFYKDCLTKYEGWACYTFRHLKPTEEYQNNIGEFFRDVAEDGYRIDFQGISDQYIHEKNEKGELHLFEIHNKDWNLDKARDGKSKTTQKNLHTLYFESLFSNDNVVQNFPIKLNGQAEIFYRPKTEKDKLESKKDKKGNKVIDHKRYSENKIFFHVPLTLNRTKNDSYRFNAQINNFLANNKDINIIGVDRGEKHLVYYSVITQASDILESGSLNELNGVNYAEKLGKKAENREQARRDWQDVQGIKDLKKGYISQVVRKLADLAIKHNAIIILEDLNMRFKQVRGGIEKSIYQQLEKALIDKLSFLVDKGEKNPEQAGHLLKAYQLSAPFETFQKMGKQTGIIFYTQASYTSKSDPVTGWRPHLYLKYFSAKKAKDDIAKFTKIEFVNDRFELTYDIKDFQQAKEYPNKTVWKVCSNVERFRWDKNLNQNKGGYTHYTNITENIQELFTKYGIDITKDLLTQISTIDEKQNTSFFRDFIFYFNLICQIRNTDDSEIAKKNGKDDFILSPVEPFFDSRKDNGNKLPENGDDNGAYNIARKGIVILNKISQYSEKNENCEKMKWGDLYVSNIDWDNFVTQANARH

SEQ ID NO: 32 - protein, 1291 aa, Unknown
  Nitrospinae bacterium RIFCSPLOWO2_02_FULL_39_110
MENKELFNSFTKKCQLSKTLRFELVPQGETEKFIKKKGLLKQDKDRADDYRKAKKLIDEYHKDFIEHALSGKQLKKLQAYYDEFTALLGKPAKERDTRVLSNISEALRKEIAGWLKDNPIKDEKKLIKEIVPNFLKSKDRNDDAALVLKFKGFTTYFGGFNENRRNMYSAEEKSTAIAYRIVHENLPKFISNMKTFQRLTENHKIDFLEVETQMNDELDGNKLKDVFSIDYFNQCLTQSGIDRYNTILGGKTDSRGAQIQGVNIKINLFGQKNNLKSKDAPVLARLYKQILSDRTSASFLPEKFETAKELIASIREFYEKAINGFIKNNERKNVIDEIESLFNKKLNPTDCDLSQIYIRNIFISAVSQQIFGSYSVITSALNAFIDKTYKTKKEKEKQQKKSYFGIAEIEAALKAYLQETEIENKEKREEVLKHANPVLQYLNSLTTNIEINNKKEKANVIVRFRQQHEAVQSVLNKEYGNNSELIQDKNAVAAIKDYLDSCQHILHFVKPLFVVPPKKENAELPDKDAFYSEFDELFEQLKSVTPLYNMVRNFLTQKPYSTEKIKLNFENVSLLSGWDVNKETDNTSVIFRKDSLYYLGIMDKKHNKVFHDIPASSDNDFYEKMNYKLLPGANKMLPKVIFSDRWIEYFNPSKELIEKYKKGTHKKGEIFSLSDCHALIDFFKTSIAKHEDWKQFNFQFSPTKTYQDINEFYREVEHQGYKISFQNVSAKYIHQLVSEGKLFLFKIYNKDFSPYSKGKPNLHTLYWKAVFDKENLKDVVVKLNGEAEVFYRPQSIPKKSKIVHKKNKPIVNKNPNNPKKQSLFKEYDIVKDKRYTEDKYHFHVPITLNFKEAKVPSENDRKKAFAYTFNKGANETLKENFPNIIGIDRGERHLAYYSLINSKGEILEQGSFNVIKNEFNGTKHETDYYASLVEKEKARDAARKSWDTIGKIKELKEGYLSQVVHKIARMMIEHNAIIVLEDLTMKFKDIRKKVERQVYQKLEKMLIDKLNYLVFKDREAYEPGGVLNAYQLAAPFTSFKDMGKQTGFIFYVPAANTSKIDYATGFMNFIYPKIEDAVEFFQKFEYIRFNPDKNYFEFAARYNNFVKDDEAKLPDEYNEPWIICTHGEQRFQYQPSYKSFKEVNVTQELQDLFSKHAIMPYAHGNDLSPQIKDKGNTSFYKGLIALLRLVLQMRYTDGKGRDFILSPVANDEGVFFNSEKAKETEPNDADANGAYHIALKGLLLLKRIKGEFKKEEKTGKKKTQKENWIDLSISNKEWYEFAQKKEYRKDH

SEQ ID NO: 33 - protein, 1271 aa, Unknown
  Planctomycetes bacterium GWC2_39_26
MNSSNDAKKDFSSFTNKYQLSKTLRFELIPQGKTREHIETKGLLQRDEHRAESYKKVKKIIDEYHKVFIEKAMKDFRLEGLENFKLLYLKKEKEDKDKKELQEVMKGLRKQIAACFSNNKDESVAAIYKNLFAKELIKKDLKDFVKEEDKELIEEFEDFTTYFTGFHENRKNIYTHEEKSTAIAYRLIHENLPSFLDNLGVYQSIKEDHPDLDFSPMEKELYEVLNGMRVKGIEEVFTLDFFNNMLTQKGIDAYNAILGGKSEKGQRKIKGVNEYIHEHRQLKGLTLRQLPNLKPLYKQILSDRESISFLPEYFEKDQELLDSIKAFYQNGLCSWEKDGKTQDVLQEIENLLLPLNEFDLEKIFIKNDISLTGISQKIFGDWSFIGDALGSYYEHKNPLPKKLTKTYEEKKEKWVTETTHFSIADIQRALDLYKTKNEEAKEKITASILCSFFGSMGEVQVAGEPKAPNLLQRIEESYGPVTDLLNTPYPENRKLAQDKAEVSKIKNFLDTVMDLLHFIKPLHPRENIQEKDVKFYNAYSTLFDQLNLITDLYNKTRNYLTKKPYSTEKIKLNFENSTLLDGWDANKETDNTSVLLRKNGFFYLAIMDKKHNRIFNQIPKLNNGEASYEKMNYKLLPLINQQLPRVFFAKSRIEYFNPSQEILKNYENDTHKKGETFNINDCHALIDFFKASLERHEDWKNFNFKFSPTTSYEDLSGFYREVEQQGYKVTFQNVPENYINRMVEEGKLYLFQIYNKDFSPFSKGKPNLHTLYWKMLFAEENLKNVVYKLNGQAEVFFRKKSLNYDEKILKTGHHAEKLKDRFNYPIISNRRFAFDKFQFHVPITLNFKATGNGNINQDVLTYLKNNPNVNIIGIDRGERHLLYVTLIDQQRNIKQQFSLNEIVNRYNGKEYATNYHAKLDAKEKDRDVARKSWGVIENIRELKEGYLSQVVHQIAGMMVEHNAVVVMEDLNFGFKRGRQKVEKQVYQKFEKMLIDKLNYMAFKNKEAAETGGVLNALQLSNKFDSFQKLGKQSGFIFYVPAWHTSKIDPATGFVDFFKPKYENIEKAKEFFGNFKSICFNRGKNWFEFDFDYNKFTPKAEDTRTQWTVCTTPEVRYVWNRNKETANGRGGYDEYLITEKMKSLFDGEDIRIRYQNGSDIKREIVGQSSGQFFKELTRLLGVTLSLRYNNGKKGDAEKDFILSPVANNKGEFFNSLKADASQPQNADANGAYHIALKGLWVLHQINEANDLKKVKLAISNKEWLQFVQRYES

SEQ ID NO: 34 - protein, 1289 aa, Unknown
  Unclassified Actinobacteria Nt197P3bin131
MKKESFERFVGVYPMSKTLKNRLIPVGRTEEFIRQRDIISQDEQRARDYEFIKERIDSFHAELIEDVLGDQNNCLDWIGLAEAIAAHKQPKSENKVESAARVTAEQATMRKAIINLFLARPDFKLLFKKELISEILPNYFQDDTEVVEVLSKFKGFTTYFTGFFENRKNVYSADELSTSIAHRIVHVNFGRYLENLSVLRRIEQEYPSALKNAEDELKTIFPDFEFKDLFILQNFNKVLSQIGIDRYNELIGGRTLEGNKKVRGINEHVNLAQQQDKADGKKQKLKLQLLHKQILSDRNTRSFIYDPIENDSEVYSAVRVMSDSLNMPNADFGGASSFERALELLQHISNYDLSHIFVPAKNLEKLSSQFLGQWSVSGDALDRCAVEKLGPASGMTKRQAESWRKSGCFSLSDICSALETSGQTLDLSLAGIRLGDLKAKAEEAYVKVLQLAEECVSGQRKLLEDENATDTLREYLDALLEFLHVLKLFEAPSEEDRDLDFYVDFDYIVAVMGDIVPLYNRVRNYVTQKPYSQEKFKLNFESPTLCAGWDINKESDNVATLLKKDGLFYLAIMNPEHRKSFIDAPEAATEDIYQKMIYKLLPGPNKMLPKVFFCASNIDFYDPSDKIMRIYEQGTFKKGDTFSLKDCHTLIDFFKDSIDRHPDWSQFGFKFSETSSYKDIAGFYTEVQQQGYKLRFVDVDASYVDKLVEEGKVYLFKIYNKDFSLHVRNREKKSSKNMHTLYLESLFSVQNLEDVVFKLNGEAELFYRRGSIKKPVIHKAGEKLVGRTYQEIDKTTGETITRHMPEKVYLELFKNLNGKLAYKSLCDKAKELLDNKKVESRVAEYDIVKDKRYTKDQYFFHVPITINFKATGSQPFNSNALEYLDGNREVKIIGIDRGERHLLYYTLMDQQGNIIDQNSLNSLNGVDYREKLHEREEERKEARKSWKRISKIADLKEGYLSLAVNKLAKMMIDNNAILVLEDLNFGFKQGRVKIEKQVYQKFEKQLIDKLNYFALKPNDVPINQPGGVLKGYQLAAPFESFEKLGKQSGFIFYVPAWNTSHIDPTTGFCDLFLWNRESTDAAGFFSRMETIRYNAESDYFEFSFDYADYHTRCLSPQTKWTVCSHGDRRWIWTARTTKTGRDYEVDEINVNQELKDIFDSAGINYHDGHDLRGDIAAQETKEFGKKMLRQFKNLLKLRYSTAQSDEEQHDCIISPVMNDNGEFFMSKEGCKNLPENADANGAYNIALKGLQLLREQIEVNEKTGLRRIAYPKKPNEAWLRFVQGKEWME

SEQ ID NO: 35 - DNA, 37 bp, Oryza sativa
tccttctcctgaggagcaagagccatcacaggcaaga

SEQ ID NO: 36 - DNA, 49 bp, Oryza sativa
acgttgagcatatggttgtaacttcagaagtaccaaatcacccacttga

SEQ ID NO: 37 - DNA, 37 bp, Solanum lycopersicum
gctttgatgtgggtgctaaatcaagaaagctacaaaa

SEQ ID NO: 38 - DNA, 37 bp, Solanum lycopersicum
tgtttggcgggataccattgtatatcttgatgatgat

SEQ ID NO: 39 - DNA, 36 bp, Artificial Sequence
  synthetic
gtctaagaactttaaataatttctactgttgtagat

SEQ ID NO: 40 - DNA, 36 bp, Artificial Sequence
  synthetic
gttgaatagccttaaagaatttctactagtgtagat

SEQ ID NO: 41 - DNA, 36 bp, Artificial Sequence
  synthetic
gtctaggtactctctttaatttctactattgtagat

SEQ ID NO: 42 - DNA, 36 bp, Artificial Sequence
  synthetic
gtctaagccttagcttaaatttctactgttgtagat

SEQ ID NO: 43 - DNA, 36 bp, Artificial Sequence
  synthetic
ctctagcaggcctggcaaatttctactgttgtagat

SEQ ID NO: 44 - DNA, 36 bp, Artificial Sequence
  synthetic
gtctaatagacctattaaatttctacttttgtagat

SEQ ID NO: 45 - DNA, 36 bp, Artificial Sequence
  synthetic
gtctaaagcaccaataaaatttctactattgtagat

SEQ ID NO: 46 - DNA, 30 bp, Artificial Sequence
  synthetic
aatagtagaaatttaaatggattactattc

SEQ ID NO: 47 - DNA, 36 bp, Artificial Sequence
  synthetic
atctacaacagtagaaattactataggttacttaac

SEQ ID NO: 48 - DNA, 36 bp, Artificial Sequence
  synthetic
atctacgacagtagaaattttataaggcctttagac

SEQ ID NO: 49 - DNA, 36 bp, Artificial Sequence
  synthetic
atctacaaaagtagaaattttatatatggattaaac

SEQ ID NO: 50 - DNA, 31 bp, Artificial Sequence
  synthetic
caaaagtagaaattttatagaggtatttggc

SEQ ID NO: 51 - DNA, 36 bp, Artificial Sequence
  synthetic
atctacaacagtagaaatcttataggtgtattcggg

SEQ ID NO: 52 - DNA, 36 bp, Artificial Sequence
  synthetic
atctacaatagtagaaatttaaaaacgtcattagac

SEQ ID NO: 53 - DNA, 36 bp, Artificial Sequence
  synthetic
atctacaaaagtagaaattctttcctctctttagag

SEQ ID NO: 54 - DNA, 34 bp, Oryza sativa
tgtttgaagaagggttatggccaatgcttggccc

SEQ ID NO: 55 - DNA, 24 bp, Artificial Sequence
  synthetic
tgtttgaagaagggttatggcccc

SEQ ID NO: 56 - DNA, 28 bp, Artificial Sequence
  synthetic
tgtttgaagaagggttatggcttggccc

SEQ ID NO: 57 - DNA, 22 bp, Artificial Sequence
  synthetic
tgtttgaagaagggttagcccc

SEQ ID NO: 58 - DNA, 27 bp, Artificial Sequence
  synthetic
tgtttgaagaagggttatggctgcccc

SEQ ID NO: 59 - DNA, 36 bp, Oryza sativa
gctctttctcctgaggagcaagagccatcacaggta

SEQ ID NO: 60 - DNA, 27 bp, Artificial Sequence
  synthetic
gctctttctcctgaggagcaacaggta

SEQ ID NO: 61 - DNA, 28 bp, Artificial Sequence
  synthetic
gctctttctcctgaggagcaagcaggta

SEQ ID NO: 62 - DNA, 34 bp, Oryza sativa
tccttctcctgaggagcaagagccatcacaggta

SEQ ID NO: 63 - DNA, 26 bp, Artificial Sequence
  synthetic
tccttctcctgaggagcaagcaggta

SEQ ID NO: 64 - DNA, 25 bp, Artificial Sequence
  synthetic
tccttctcctgaggagcaacaggta

(19) United States
(12) Patent Application Publication
(10) Pub. No.: US 2025/0340895 A1
(43) Pub. Date: Nov. 6, 2025

(54) GENOME EDITING IN PLANTS USING CAS12A NUCLEASES

(71) Applicant: University of Maryland, College Park, College Park, MD (US)

(72) Inventors: YIPING QI, Potomoc, MD (US); YINGXIAO ZHANG, Raleigh, NC (US); MICAH DAILEY, Durham, NC (US); GEN LI, Greenbelt, MD (US)

(21) Appl. No.: 18/558,934

(22) PCT Filed: May 6, 2022
(86) PCT No.: PCT/US2022/028099

§ 371 Date: Nov. 3, 2023

Related U.S. Application Data
(60) Provisional application No. 63/186,054, filed on May 7, 2021.

(51) Int. Cl. C12N 15/82; C12N 9/12; C12N 9/22; C12N 9/78; C12N 15/11

(52) U.S. Cl. CPC C12N 15/8201; C12N 9/1276; C12N 9/226; C12N 9/78; C12N 15/111; C07K 2319/80; C12N 2310/20; C12N 2800/22; C12Y 207/07049; C12Y 305/04

(57) ABSTRACT

Compositions and methods for modifying genomic DNA sequences of a plant cell are provided. The methods produce double stranded breaks at target sites in a genomic DNA sequence, resulting in mutation, insertion, and/or deletion of DNA sequences at the target site(s) in a genome. The compositions comprise nucleic acid constructs comprising nucleotide sequences that encode a Cas12a protein. The nucleic acid constructs can be used to direct the modification of genomic DNA at a target site. Methods to use these DNA constructs to modify genomic DNA sequences are also provided.

### CROSS REFERENCE TO RELATED APPLICATIONS

This application claims priority to provisional application U.S. Ser. No. 63/186,054, filed May 7, 2021, which hereby is incorporated herein by reference in its entirety.

### SEQUENCE LISTING

The instant application contains a sequence listing which has been submitted in ASCII format by electronic submission and is hereby incorporated by reference in its entirety. Said ASCII copy, created on Apr. 27, 2022, is named P13842W000_ST25.txt and is 286,334 bytes in size.

### TECHNICAL FIELD

The present disclosure relates to compositions and methods for editing genomic sequences in plants.

### BACKGROUND

Clustered Regularly Interspaced Short Palindromic Repeats (CRISPR) is the predominant technology to generate targeted mutagenesis in living organisms. CRISPR and its derived technologies have been widely used for genome editing, transcriptional regulation, epigenetic modification, and genomic region visualization and isolation. CRISPR associated protein 12a (Cas12a) belongs to the Class II Type V CRISPR system and is the second most used CRISPR system in plants. Cas12a has a T-rich protospacer adjacent motif (PAM) requirement and generates staggered DNA double-strand break (DSB) ends distal from the PAM, resulting in large deletions and high editing efficiency at AT-rich genomic regions. Moreover, Cas12a only requires a short CRISPR RNA (crRNA) for target site recognition, making it an ideal platform for multiplexed genetic engineering and ribonucleoprotein (RNP) delivery. There is a need in the art for improved Cas12a systems that can reliably and efficiently generate targeted mutagenesis at ambient temperatures and in diverse plant species.

### SUMMARY

The presently disclosed subject matter relates generally to genome engineering. In certain embodiments, the disclosed subject matter relates to compositions and methods for editing genome sequences in a plant cell.

In certain embodiments, the compositions relate to CRISPR Cas12a nucleases, for example, Eubacterium ventriosum Cas12a (Ev1Cas12a), Hydrogenovibrio sp. XS5 (Hs1Cas12a), or Porphyromonas cansulci (Pc1Cas12a). Applicants have surprisingly found that Ev1Cas12a, Hs1Cas12a, and Pc1Cas12a provide superior editing efficiency in diverse plant cells from among 17 Cas12a nucleases tested. The methods produce double-stranded breaks (DSBs) at a target site in a genomic DNA sequence, resulting in mutation, insertion, and/or deletion of DNA sequences at the target site in a genome. In certain embodiments, the methods may include multiplexed genome editing.

Compositions comprise nucleic acid constructs comprising a nucleotide sequence that encodes a Cas12a protein operably linked to a promoter that is operable in the cells of interest. Particular Cas12a protein sequences are set forth in SEQ ID NOs: 18-34; particular Cas12a protein-encoding nucleotide sequences are set forth in SEQ ID NOs: 1-17. The nucleic acid constructs comprising a sequence that encodes the Cas12a proteins of the disclosure, or the Cas12a proteins of the disclosure themselves, can be used to direct the modification of genomic DNA at genomic loci. Methods to use these DNA constructs to modify genomic DNA sequences are described herein. In certain embodiments, the nucleic acid constructs are vectors for delivery of Cas12a to plant cells. Modified plants and plant cells are also encompassed.

Compositions and methods for modulating the expression of genes are also provided. The methods target protein(s) to sites in a genome to effect an up- or down-regulation of a gene or genes whose expression is regulated by the targeted site in the genome. Compositions comprise a nucleic acid construct comprising a nucleotide sequence that encodes a Cas12a protein with diminished or abolished nuclease activity, optionally fused to a transcriptional activation or repression domain. Methods to use these nucleic acid constructs to modify gene expression are described herein.

While multiple embodiments are disclosed, still other embodiments will become apparent to those skilled in the art from the following detailed description, which shows and describes illustrative embodiments. Accordingly, the figures and detailed description are to be regarded as illustrative in nature and not restrictive.

### BRIEF DESCRIPTION OF THE FIGURES

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 shows a phylogenetic tree of Cas12a nucleases by Clustal Omega. Amino acid sequences of Cas12a ortholog candidates were acquired from Joint Genome Institute Microbial Genomes and Microbiomes (JGI IMG/M) database by using LbCas12a, FnCas12a. ErCas12a and Mb2Cas12a as the blast queries. Homology of the Cas12a nucleases to the queries was analyzed via neighbor-joining phylogenetic tree in cladogram on the European Bioinformatics Institute (EBI) server.

FIG. 2A-F shows screening of ten novel Cas12a orthologs in rice protoplasts. Targeted mutagenesis efficiencies (percentage) of ten novel Cas12a orthologs at six target sites with TTTV and VTTV PAMs in rice protoplasts. WT, protoplasts transformed with water. LbCas12a and Mb2Cas12a are used as controls. Editing efficiencies are measured using Restriction Fragment Length Polymorphism (RFLP) analysis. Data are presented as mean values±SEM. n=3 biologically independent samples.

FIG. 3A-D shows screening of seven novel Cas12a orthologs in rice protoplasts. Targeted mutagenesis efficiencies (percentage) of 7 novel Cas12a orthologs at four target sites with TTTV and VTTV PAMs in rice protoplasts. WT, protoplasts transformed with water. Data are presented as mean values±SEM. n=3 biologically independent samples.

FIG. 4A-G shows genome editing of three novel Cas12a orthologs in rice protoplasts. Total mutation and deletion efficiencies (percentage) of three novel Cas12a orthologs at seven target sites with TTTV and VTTV PAMs at 32° C. and 22° C. in rice protoplasts. WT, protoplasts transformed with water. LbCas12a and Mb2Cas12a are used as controls. Editing efficiencies are measured using next-generation sequencing (NGS) of PCR amplicons. Data are presented as mean values±SEM. n=3 biologically independent samples.

FIG. 5A-G shows deletion size profiles of EvCas12a in rice protoplasts at 32° C. and 25° C. Frequencies (in percentage shown in the y-axis) are calculated using the number of reads with N bp deletions divided by the number of all the reads with deletions. Data are presented as mean values±SEM. n=3 biologically independent samples.

FIG. 6A-G shows deletion size profiles of HsCas12a in rice protoplasts at 32° C. and 25° C. Frequencies (in percentage shown in the y-axis) are calculated using the number of reads with N bp deletions divided by the number of all the reads with deletions. Data are presented as mean values±SEM. n=3 biologically independent samples.

FIG. 7A-G shows deletion size profiles of PclCas12a in rice protoplasts at 32° C. and 25° C. Frequencies (in percentage shown in the y-axis) are calculated using the number of reads with N bp deletions divided by the number of all the reads with deletions. Data are presented as mean values±SEM. n=3 biologically independent samples.

FIG. 8A-D shows deletion position profiles of ExCas12a, HsCas12a, and Pc1Cas12a in rice protoplasts at 32° C. and 25° C. Frequencies (in percentage shown in the y-axis) are calculated using the number of reads with deletions at each nucleotide position divided by the number of all the reads with deletions. PAM sequence (solid line) and protospacer sequence (dashed line) are underlined (SEQ ID NO: 35 and 36). Data are presented as mean values±SEM. n=3 biologically independent samples (n=1 for WT).

FIG. 9A-F shows genome editing of ExCas12a, HsCas12a, and PclCas12a in tomato protoplasts at 32° C. and 25° C. Total mutation and deletion efficiencies (percentage) of the three Cas12a orthologs at six target sites with TTTV PAMs at 32° C. and 22° C. in tomato protoplasts are shown. WT, protoplasts transformed with water. LbCas12a and Mb2Cas12a are used as controls. Editing efficiencies are measured using next-generation sequencing (NGS) of PCR amplicons. Data are presented as mean values±SEM. n=3 biologically independent samples.

FIG. 10A-F shows deletion size profiles of EvCas12a in tomato protoplasts. Frequencies (in percentage shown in the y-axis) are calculated using the number of reads with N bp deletions divided by the number of all the reads with deletions. Data are presented as mean values±SEM. n=3 biologically independent samples.

FIG. 11A-F shows deletion size profiles of HsCas12a in tomato protoplasts. Frequencies (in percentage shown in the y-axis) are calculated using the number of reads with N bp deletions divided by the number of all the reads with deletions. Data are presented as mean values±SEM. n=3 biologically independent samples.

FIG. 12A-F shows deletion size profiles of Pc1Cas12a in tomato protoplasts. Frequencies (in percentage shown in the y-axis) are calculated using the number of reads with N bp deletions divided by the number of all the reads with deletions. Data are presented as mean values±SEM. n=3 biologically independent samples.

FIG. 13A-D shows deletion position profiles of ExCas12a, HsCas12a, and Pe1Cas12a in tomato protoplasts. Frequencies (in percentage shown in the y-axis) are calculated using the number of reads with deletions at each nucleotide position divided by the number of all the reads with deletions. PAM sequence (solid line) and protospacer sequence (dashed line) are underlined (SEQ ID NO: 37 and 38). Data are presented as mean values±SEM. n=3 biologically independent samples (n=1 for WT).

FIG. 14A-E shows genome editing of ExCas12a, HsCas12a, and Pc1Cas12a in rice protoplasts at low temperature. Total mutation and deletion efficiencies (percentage) of two novel Cas12a orthologs at five target sites with TTTV and VTTV PAMs at 28° C., 25° C. and 22° C. in rice protoplasts. WT, protoplasts transformed with water. LbCas12a and Mb2Cas12a are used as controls. Editing efficiencies are measured using next-generation sequencing (NGS) of PCR amplicons. Data are presented as mean values±SEM. n=3 biologically independent samples.

FIG. 15A-F shows genome editing of ExCas12a, HsCas12a, and Pc1Cas12a in tomato protoplasts at low temperature. Total mutation and deletion efficiencies (percentage) of two novel Cas12a orthologs at six target sites with TTTV PAMs at 28° C., 25° C. and 22° C. in tomato protoplasts. WT, protoplasts transformed with water. LbCas12a and Mb2Cas12a are used as controls. Editing efficiencies are measured using next-generation sequencing (NGS) of PCR amplicons. Data are presented as mean values±SEM. n=3 biologically independent samples.

FIG. 16A-C shows genome editing of Hs1Cas12a and Ev1Cas12a in poplar T0 transgenic plants. Indel frequency of Ps1Cas12a and Ev1Cas12a compared with LbCas12a, Mb2Cas12a, and AsCas12a at six target sites in poplar T0 plants is shown.

FIG. 17 shows putative CRISPR RNA sequences for Cas12a orthologs. The CRISPR RNA (crRNA) sequences of 14 Cas12a orthologs of the disclosure are aligned with crRNA of FnCas12a (SEQ ID NOs: 39-53). The sequences in bold are predicted mature crRNAs.

### DETAILED DESCRIPTION

The present disclosure relates to Cas12a-mediated genome editing in plants. Methods and compositions are provided herein for the control of gene expression involving sequence targeting, such as genome perturbation or gene-editing, that relate to the CRISPR-Cas12a system and components thereof. The methods and compositions include nucleic acids to bind target DNA sequences. Also provided are nucleic acids encoding the Cas12a polypeptides, as well as methods of using Cas12a polypeptides to modify chromosomal (i.e., genomic) or organellar DNA sequences of host cells including plant cells. The Cas12a polypeptides interact with specific guide RNAs (gRNAs), which direct the Cas12a endonuclease to a specific target site, at which site the Cas12a endonuclease introduces a double-stranded break that can be repaired by a DNA repair process such that the DNA sequence is modified. The methods disclosed herein can be used to target and modify specific chromosomal sequences and/or introduce exogenous sequences at targeted locations in the genome of plant cells. The methods can further be used to introduce sequences or modify regions within organelles (e.g., chloroplasts and/or mitochondria). Furthermore, the targeting is specific with limited off target effects.

Unless defined otherwise, all technical and scientific terms used herein have the same meaning as commonly understood by one skilled in the art to which embodiments of the disclosure pertain. Many methods and materials similar, modified, or equivalent to those described herein can be used in the practice of the embodiments of the present disclosure without undue experimentation, the preferred materials and methods are described herein.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicates otherwise. The word “or” means any one member of a particular list and also includes any combination of members of that list. Further, all units, prefixes, and symbols may be denoted in their SI accepted form.

Numeric ranges recited within the specification are inclusive of the numbers defining the range and include each integer within the defined range. Throughout this disclosure, various aspects are presented in a range format. It should be understood that the description in range format is merely for convenience and brevity and should not be construed as an inflexible limitation on the scope of the present disclosure or the associated claims. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 11/2, and 43/4. This applies regardless of the breadth of the range.

### Cas12a Endonucleases

The terms “CRISPR-Cas protein”, “CRISPR protein”, “Cas protein”, “Cas effector protein”, “CRISPR enzyme”, and “Cas enzyme” may be used interchangeably herein. Provided herein are Cas12a proteins, and fragments and variants thereof, for use in modifying genomes including plant genomes. The present disclosure encompasses the use of a Cas12a effector protein, derived from a Cas12a locus denoted as subtype V-A. Such effector proteins are also referred to as Cpf1. Cas12b proteins contain a RuvC-like nuclease domain homologous to the corresponding domain of Cas9. However, Cas12a lacks the HNH nuclease domain that is present in all Cas9 proteins, and the RuvC-like domain is contiguous in the Cas12a sequence, in contrast to Cas9 where it contains long inserts including the HNH domain. Accordingly, in particular embodiments, the CRISPR-Cas enzyme comprises only a RuvC-like nuclease domain. Cas12a creates a staggered cut at the target locus, with a 5′ overhang, or a “sticky end” at the PAM distal side of the target sequence. Cas12a creates double strand breaks at the distal end of PAM, in contrast to cleavage at the proximal end of PAM created by Cas9. Cas12a and Cas12b are both Type V CRISPR-Cas proteins that share structure similarity. Unlike Cas9, which generates blunt cuts at the proximal end of PAM, Cas12a and Cas12b generate staggered cuts at the distal end of PAM. Examples of Cas12a polypeptides of the disclosure are set forth in SEQ ID NOs: 18-34 and summarized in Table 1.

TABLE 1

| Cas12a | Nucleotide | Amino acid | |
|---|---|---|---|
| name | sequence | sequence | Organism |
| Ba1Cas12a | SEQ ID NO: 1 | SEQ ID NO: 18 | Brumimicrobium aurantiacum |
| Ca1Cas12a | SEQ ID NO: 2 | SEQ ID NO: 19 | Saccharobesus litoralis |
| | | | (Catenovulum sp. CCB-QB4) |
| Cb1Cas12a | SEQ ID NO: 3 | SEQ ID NO: 20 | Clostridiales bacterium RUG149 |
| Cc1Cas12a | SEQ ID NO: 4 | SEQ ID NO: 21 | Candidatus Campbellbacteria bacterium |
| | | | RIFCSPLOWO2_01_FULL_34_15 |
| Cu1Cas12a | SEQ ID NO: 5 | SEQ ID NO: 22 | Candidatus Uhrbacteria bacterium |
| | | | CG11_big_fil_rev_8_21_14_0_20_41_9 |
| Ev1Cas12a | SEQ ID NO: 6 | SEQ ID NO: 23 | Eubacterium ventriosum |
| Hs1Cas12a | SEQ ID NO: 7 | SEQ ID NO: 24 | Hydrogenovibrio sp. XS5 |
| Pa2Cas12a | SEQ ID NO: 8 | SEQ ID NO: 25 | Parcubacteria bacterium |
| | | | JGI MDM2 000213CP-K14 |
| Pc1Cas12a | SEQ ID NO: 9 | SEQ ID NO: 26 | Porphyromonas cansulci JCM 13913 |
| Sc1Cas12a | SEQ ID NO: 10 | SEQ ID NO: 27 | Sedimentisphaera cyanobacteriorum |
| Bb1Cas12a | SEQ ID NO: 11 | SEQ ID NO: 28 | Bdellovibrionales bacterium SP5OBV1 |
| Cd1Cas12a | SEQ ID NO: 12 | SEQ ID NO: 29 | Candidate division WWE3 bacterium |
| | | | CG10_big_fil_rev_8_21_14_0_10_35_32 |
| CW1Cas12a | SEQ ID NO: 13 | SEQ ID NO: 30 | Candidatus Woesearchaeota archaeon |
| | | | CG10_big_fil_rev_8_21_14_0_10_37_12 |
| Mb1Cas12a | SEQ ID NO: 14 | SEQ ID NO: 31 | Candidatus Roizmanbacteria |
| | | | bacterium GW2011_GWA2_37_7 |
| Nb1Cas12a | SEQ ID NO: 15 | SEQ ID NO: 32 | Nitrospinae bacterium |
| | | | RIFCSPLOWO2_02_FULL_39_110 |
| Pb1Cas12a | SEQ ID NO: 16 | SEQ ID NO: 33 | Planctomycetes bacterium GWC2_39_26 |
| Ua1Cas12a | SEQ ID NO: 17 | SEQ ID NO: 34 | Unclassified Actinobacteria |
| | | | Nt197P3bin131 |

In particular embodiments, the Cas12a protein is from a species selected from Brumimicrobium aurantiacum, Saccharobesus litoralis, Clostridiales bacterium RUG149, Candidatus Campbellbacteria bacterium RIFCSPLOWO2_01_FULL_34_15, Candidatus Uhrbacteria bacterium CG11_big_fil_rev_8_21_14_0_20_41_9, Eubacterium ventriosum, Hydrogenovibrio sp. XS5, Parcubacteria bacterium JGI MDM2 000213CP-K14, Porphyromonas cansulci JCM 13913, Sedimentisphaera cyanobacteriorum, Bdellovibrionales bacterium SP5OBV1, Candidate division WWE3 bacterium CG10_big_fil_rev_8_21_14_0_10_35_32, Candidatus Woesearchaeota archaeon CG10_big_fil_rev_8_21_14_0_10_37_12, Candidatus Roizmanbacteria bacterium GW2011_GWA2_37_7, Nitrospinae bacterium RIFCSPLOWO2_02_FULL_39_110, Planctomycetes bacterium GWC2_39_26, and Unclassified Actinobacteria Nt197P3bin131.

Cas12a polypeptides can be wild type Cas12a polypeptides, modified Cas12a polypeptides, or a fragment of a wild type or modified Cas12a polypeptide. The Cas12a polypeptide can be modified to increase nucleic acid binding affinity and/or specificity, alter an enzymatic activity, improve temperature sensitivity, alter PAM requirements and/or change another property of the protein. For example, nuclease (i.e., DNase, RNase) domains of the Cas12a polypeptide can be modified, deleted, or inactivated. Alternatively, the Cas12a polypeptide can be truncated to remove domains that are not essential for the function of the protein.

In some embodiments, the Cas12a polypeptide can be derived from a wild type Cas12a polypeptide or fragment thereof. In other embodiments, the Cas12a polypeptide can be derived from a modified Cas12a polypeptide. For example, the amino acid sequence of the Cas12a polypeptide can be modified to alter one or more properties (e.g., nuclease activity, affinity, stability, etc.) of the protein. Alternatively, domains of the Cas12a polypeptide not involved in RNA-guided cleavage can be eliminated from the protein such that the modified Cas12a polypeptide is smaller than the wild type Cas12a polypeptide.

In some embodiments, the Cas12a polypeptide can be modified to inactivate the nuclease domain so that it is no longer functional. In some embodiments in which one of the nuclease domains is inactive, the Cas12a polypeptide does not cleave double-stranded DNA. The nuclease domain can be modified using well-known methods, such as site-directed mutagenesis, PCR-mediated mutagenesis, and total gene synthesis, as well as other methods known in the art. Cas12a proteins with inactivated nuclease domains (dCas12a proteins) can be used to modulate gene expression without modifying DNA sequences. In certain embodiments, a dCas12a protein may be targeted to particular regions of a genome such as promoters for a gene or genes of interest through the use of appropriate gRNAs. The dCas12a protein can bind to the desired region of DNA and may interfere with RNA polymerase binding to this region of DNA and/or with the binding of transcription factors to this region of DNA. This technique may be used to up- or down-regulate the expression of one or more genes of interest. In certain other embodiments, the dCas12a protein may be fused to a repressor domain to further downregulate the expression of a gene or genes whose expression is regulated by interactions of RNA polymerase, transcription factors, or other transcriptional regulators with the region of chromosomal DNA targeted by the gRNA. In certain other embodiments, the dCas12a protein may be fused to an activation domain to effect an upregulation of a gene or genes whose expression is regulated by interactions of RNA polymerase, transcription factors, or other transcriptional regulators with the region of chromosomal DNA targeted by the gRNA.

The Cas12a polypeptides disclosed herein can further comprise at least one nuclear localization signal (NLS). In general, an NLS comprises a stretch of basic amino acids. Nuclear localization signals are known in the art (see, e.g., Lange et al., J. Biol. Chem. (2007) 282:5101-5105). The NLS can be located at the N-terminus, the C-terminus, or in an internal location of the Cas12a polypeptide.

The Cas12a polypeptide disclosed herein can further comprise at least one plastid targeting signal peptide, at least one mitochondrial targeting signal peptide, or a signal peptide targeting the Cas12a polypeptide to both plastids and mitochondria. Plastid, mitochondrial, and dual-targeting signal peptide localization signals are known in the art (see, e.g., Nassoury and Morse (2005) Biochim Biophys Acta 1743:5-19: Kunze and Berger (2015) Front Physiol 6:259: Herrmann and Neupert (2003) IUBMB Life 55:219-225: Soll (2002) Curr Opin Plant Biol 5:529-535: Carrie and Small (2013) Biochim Biophys Acta 1833:253-259; Carrie et al. (2009) FEBSJ 276:1187-1195: Silva-Filho (2003) Curr Opin Plant Biol 6:589-595: Peeters and Small (2001) Biochim Biophys Acta 1541:54-63: Murcha et al. (2014) J Exp Bot 65:6301-6335; Mackenzie (2005) Trends Cell Biol 15:548-554; Glaser et al. (1998) Plant Mol Biol 38:311-338). The plastid, mitochondrial, or dual-targeting signal peptide can be located at the N-terminus, the C-terminus, or in an internal location of the Cas12a polypeptide.

In still other embodiments, the Cas12a polypeptide can also comprise at least one marker domain. Non-limiting examples of marker domains include fluorescent proteins, purification tags, and epitope tags. In certain embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellowl), blue fluorescent proteins (e.g. EBFP, EBFP2, Azurite, mKalamal, GFPuv, Sapphire, T-sapphire), cyan fluorescent proteins (e.g. ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, 51, T7, V5, VSV-G, 6×His, biotin carboxyl carrier protein (BCCP), and calmodulin.

In certain embodiments, the Cas12a polypeptide may be part of a protein-RNA complex comprising a guide RNA. The guide RNA interacts with the Cas12a polypeptide to direct the Cas12a polypeptide to a specific target site, wherein the 5′ end of the guide RNA can base pair with a specific protospacer sequence of the nucleotide sequence of interest in the plant genome, whether part of the nuclear, plastid, and/or mitochondrial genome. As used herein, the term “DNA-targeting RNA” refers to a guide RNA that interacts with the Cas12a polypeptide and the target site of the nucleotide sequence of interest in the genome of a cell. A DNA-targeting RNA, or a DNA polynucleotide encoding a DNA-targeting RNA, can comprise: a first segment comprising a nucleotide sequence that is complementary to a sequence in the target DNA, and a second segment that interacts with a Cas12a polypeptide. In certain embodiments, the DNA-targeting RNA comprises the nucleotide sequence set forth in any of SEQ ID NOs: 39-53.

The polynucleotides encoding Cas12a polypeptides disclosed herein can be used to isolate corresponding sequences from other prokaryotic or eukaryotic organisms, or from metagenomically-derived sequences whose native host organism is unclear or unknown. In this manner, methods such as PCR, hybridization, and the like can be used to identify such sequences based on their sequence homology or identity to the sequences set forth herein. Sequences isolated based on their sequence identity to the entire Cas12a sequences set forth herein or to variants and fragments thereof are encompassed by the present disclosure. Such sequences include sequences that are orthologs of the disclosed Cas12a sequences. “Orthologs” is intended to mean genes derived from a common ancestral gene and which are found in different species as a result of speciation. Genes found in different species are considered orthologs when their nucleotide sequences and/or their encoded protein sequences share at least about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode polypeptides having Cas12a endonuclease activity and which share at least about 75% or more sequence identity to the sequences disclosed herein, are encompassed by the present disclosure.

Examples of Ev1Cas12a ortholog species include, for example, Eubacterium rectale (OM05-3AA), Lachnospiraceae bacterium NSJ-46, Eubacterium eligens (ATCC 27750). Lachnospira eligens (2789STDY5834878), Bacteroides galacturonicus (DSM 3978), Lachnospira pectinoschiza (2789STDY5834886), Coprococcus eutactus (2789STDY5608843), Coprococcus sp. AF16-5, Coprococcus sp. BIOML-A2, Coprococcus sp. AF19-8AC, Coprococcus sp. AF16-22, Synergistes jonesii (78-1), Candidatus Methanoplasma termitum Mpt1, Treponema porcinum (ATCC BAA-908), Fibrobacter sp. UWH8, Pseudobutyrivibrio ruminis (CF1b), and Phocaeicola plebeius (AF25-1 LB), Prevotella disiens (FB035-09AN), Prevotella ihumii (Marseille-P3385), and Prevotella stercorea (AF42-9).

Examples of Hs1Cas12a ortholog species include, for example, Sphingobacteriales bacterium EBPR_Bin_354, Smithella sp. SCADC, unclassified Bacteroidales bin 22, Smithella sp. SC_K08D17, Francisella novicida (TCH2015, PA10-7858, Fx1), Bacteroidetes bacterium GWF2_33_38, Planctomycetes bacterium GWC2_39_26, Flavobacterium sp. 316, Prevotella albensis DSM 11370, unclassified Prevotellaceae bin 38, Flavobacterium branchiophilum (FL-15), Prevotellaceae bacterium RUG488, Bacteroidetes oral taxon 274 F0058, Moraxella lacunata (CCUG 57757A), Sulfurimonas crateris (SN118), Phocaeicola plebeius (AF27-1), Moraxella caprae (ERS1429836), Leptospira ryugenii (YH101), Moraxella bovis (CCUG 2133), Nitrospinae bacterium RIFCSPLOWO2_02_FULL_39_110, Porphyromonas cansulci (JCM 13913), Porphyromonas crevioricanis (NCTC 12858), Barnesiella sp. An22, Moraxella bovoculi (23343), Moraxella ovis (NCTC 11019), Prevotella bryantii (B14), Prevotella copri (AF15-25), Moraxella equi (CCUG 4950), Eubacterium coprostanoligenes (ATCC 51222), Sedimentisphaera cyanobacteriorum (L21-RPul-D3), Prevotella sp. bin 35, Moraxella sp. VT-16-12, Candidatus Woesearchaeota archaeon CG10_big_fil_rev_8_21_14_0_10_37_12, Leptospira inadai (sv. Lyme 10), Lachnospiraceae bacterium COE1, Bacteroidales bacterium KA00251, Prevotella sp. RUG176, Butyrivibrio sp. NC3005, Pseudobutyrivibrio xylanivorans (MA3014 v2), Sodaliphilus pleomorphus (Oil-RF-744-WCA-WT-10), Pseudobutyrivibrio ruminis (CF1b), Clostridiales bacterium RUG149, Synergistes jonesii (78-1), Oribacterium sp. NK2B42, Lachnospiraceae bacterium MA2020, Prevotella sp. bin 29, Brumimicrobium aurantiacum (N62), Francisella hispaniensis (CCUG 58020), Ruminococcus sp. AF37-3AC, Bacillus sp. OAE603, Ruminococcus bromii (AF25-7 LB), Ruminococcus sp. AM36-18, Ruminococcus sp. AM28-29 LB, Arcobacter butzleri (L348), Bdellovibrionales bacterium SP5OBV1, Prevotella sp. PMUR, Fibrobacter sp. UWH8, and Prevotella sp. P4-119.

As used herein. Cas12a endonuclease activity refers to CRISPR endonuclease activity wherein, a guide RNA (gRNA) associated with a Cas12a polypeptide causes the Cas12a-gRNA complex to bind to a predetermined nucleotide sequence that is complementary to the gRNA; and wherein Cas12a activity can introduce a double-stranded break at or near the site targeted by the gRNA. In certain embodiments, this double-stranded break may be a staggered DNA double-stranded break. As used herein a “staggered DNA double-stranded break” can result in a double strand break with about 1, about 2, about 3, about 4, about 5, about 6, about 7, about 8, about 9, or about 10 nucleotides of overhang on either the 3′ or 5′ ends following cleavage. In specific embodiments, the Cas12a polypeptide introduces a staggered DNA double-stranded break with a 5′ overhang. The double strand break can occur at or near the sequence to which the DNA-targeting RNA (e.g., guide RNA) sequence is targeted.

Fragments and variants of the Cas12a polynucleotides and Cas12a amino acid sequences encoded thereby that retain Cas12a nuclease activity are encompassed herein. “Cas12a nuclease activity” generally refers to the binding of a predetermined DNA sequence as mediated by a guide RNA. In embodiments wherein the Cas12a nuclease retains a functional RuvC domain. Cas12a nuclease activity can further comprise double-strand break induction. By “fragment” is intended a portion of the polynucleotide or a portion of the amino acid sequence. “Variants” is intended to mean substantially similar sequences. For polynucleotides, a variant comprises a polynucleotide having deletions (i.e., truncations) at the 5′ and/or 3′ end: deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular polynucleotide of the disclosure will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

“Variant” amino acid or protein is intended to mean an amino acid or protein derived from the native amino acid or protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein. Biologically active variants of a native polypeptide will have at least about 80%, 85%, 90%, 91%, 92%, 93%, 94%, 95%, 96%, 97%, 98%, 99% or more sequence identity to the amino acid sequence for the native sequence as determined by sequence alignment programs and parameters described herein. A biologically active variant of a protein of the disclosure may differ from that protein by as few as 1-15 amino acid residues, as few as 1-10, such as 6-10, as few as 5, as few as 4, 3, 2, or even 1 amino acid residue.

Variant sequences may also be identified by analysis of existing databases of sequenced genomes. In this manner, corresponding sequences can be identified and used in the methods of the disclosure.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17: the local alignment algorithm of Smith et al. (1981) Adv. Appl. Math. 2:482: the global alignment algorithm of Needleman and Wunsch (1970) J Mol. Biol. 48:443-453: the search-for-local alignment method of Pearson and Lipman (1988) Proc. Natl. Acad. Sci. 85:2444-2448: the algorithm of Karlin and Altschul (1990) Proc. Natl. Acad. Sci. USA 87:2264-2268, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.): the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244: Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The MUSCLE algorithm for multiple sequence alignment may be used for comparisons of multiple nucleic acid or protein sequences (Edgar (2004) Nucleic Acids Research 32:1792-1797). The BLAST programs of Altschul et al (1990) J Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the disclosure. BLAST protein searches can be performed with the BLASTX program, score=50, wordlength=3, to obtain amino acid sequences homologous to a protein or polypeptide of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See the website at ncbi.nlm.nih.gov. Alignment may also be performed manually by inspection.

The nucleic acid molecules encoding Cas12a polypeptides, or fragments or variants thereof, can be codon optimized for expression in a plant of interest or other cell or organism of interest. A “codon-optimized gene” is a gene having its frequency of codon usage designed to mimic the frequency of preferred codon usage of the host cell. Nucleic acid molecules can be codon optimized, either wholly or in part. Because any one amino acid (except for methionine and tryptophan) is encoded by a number of codons, the sequence of the nucleic acid molecule may be changed without changing the encoded amino acid. Codon optimization is when one or more codons are altered at the nucleic acid level such that the amino acids are not changed but expression in a particular host organism is increased. Those having ordinary skill in the art will recognize that codon tables and other references providing preference information for a wide range of organisms are available in the art (see, e.g., Zhang et al. (1991) Gene 105:61-72: Murray et al. (1989) Nucl. Acids Res. 17:477-508). Methodology for optimizing a nucleotide sequence for expression in a plant is provided, for example, in U.S. Pat. No. 6,015,891, and the references cited therein.

### Fusion Proteins

Fusion proteins are provided herein comprising a Cas12a polypeptide, or a fragment or variant thereof, and an effector domain. The Cas12a polypeptide can be directed to a target site by a guide RNA, at which site the effector domain can modify or effect the targeted nucleic acid sequence. The effector domain can be, for example, a cleavage domain, an epigenetic modification domain, a transcriptional activation domain, a transcriptional repressor domain, a deaminase domain, or a reverse transcriptase. The fusion protein can further comprise at least one additional domain chosen from a nuclear localization signal, plastid signal peptide, mitochondrial signal peptide, signal peptide capable of protein trafficking to multiple subcellular locations, a cell-penetrating domain, or a marker domain, any of which can be located at the N-terminus, C-terminus, or an internal location of the fusion protein. The Cas12a polypeptide can be located at the N-terminus, the C-terminus, or in an internal location of the fusion protein. The Cas12a polypeptide can be directly fused to the effector domain, or can be fused with a linker. In specific embodiments, the linker sequence fusing the Cas12a polypeptide with the effector domain can be at least 1, 2, 3, 4, 5, 6, 7, 8, 9, 10, 15, 20, 25, 30, 40, or 50 amino acids in length. For example, the linker can range from 1-5, 1-10, 1-20, 1-50, 2-3, 3-10, 3-20, 5-20, or 10-50 amino acids in length.

In some embodiments, the Cas12a polypeptide of the fusion protein can be derived from a wild type Cas12a protein. The Cas12a-derived protein can be a modified variant or a fragment. In some embodiments, the Cas12a polypeptide can be modified to contain a nuclease domain (e.g. a RuvC or RuvC-like domain) with reduced or eliminated nuclease activity. For example, the Cas12a-derived polypeptide can be modified such that the nuclease domain is deleted or mutated such that it is no longer functional (i.e., the nuclease activity is absent). A Cas12a polypeptide having a mutation in its nuclease active site, and therefore, no longer comprising nuclease activity, is commonly referred to as deadCas12a (e.g., dCas12a).

The nuclease domain can be inactivated by one or more deletion mutations, insertion mutations, and/or substitution mutations using known methods, such as site-directed mutagenesis, PCR-mediated mutagenesis, and total gene synthesis, as well as other methods known in the art. In an exemplary embodiment, the Cas12a polypeptide of the fusion protein is modified by mutating the RuvC-like domain such that the Cas12a polypeptide has no nuclease activity.

The fusion protein also comprises an effector domain located at the N-terminus, the C-terminus, or in an internal location of the fusion protein. In some embodiments, the effector domain is a cleavage domain. As used herein, a “cleavage domain” refers to a domain that cleaves DNA. The cleavage domain can be obtained from any endonuclease or exonuclease. Non-limiting examples of endonucleases from which a cleavage domain can be derived include, but are not limited to, restriction endonucleases and homing endonucleases. See, for example, New England Biolabs Catalog or Belfort et al. (1997) Nucleic Acids Res. 25:3379-3388. Additional enzymes that cleave DNA are known (e.g., 51 Nuclease: mung bean nuclease: pancreatic DNase I: micrococcal nuclease: yeast HO endonuclease). See also Linn et al. (eds.) Nucleases, Cold Spring Harbor Laboratory Press, 1993. One or more of these enzymes (or functional fragments thereof) can be used as a source of cleavage domains.

In some embodiments, the cleavage domain can be derived from a type II-S endonuclease. Type II-S endonucleases cleave DNA at sites that are typically several base pairs away from the recognition site and, as such, have separable recognition and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MbolI, and SapI.

In certain embodiments, the type II-S cleavage can be modified to facilitate dimerization of two different cleavage domains (each of which is attached to a Cas12a polypeptide or fragment thereof). In embodiments wherein the effector domain is a cleavage domain the Cas12a polypeptide can be modified as discussed herein such that its endonuclease activity is eliminated. For example, the Cas12a polypeptide can be modified by mutating the RuvC-like domain such that the polypeptide no longer exhibits endonuclease activity.

In other embodiments, the effector domain of the fusion protein can be an epigenetic modification domain. In general, epigenetic modification domains alter histone structure and/or chromosomal structure without altering the DNA sequence. Changes in histone and/or chromatin structure can lead to changes in gene expression. Examples of epigenetic modification include, without limit, acetylation or methylation of lysine residues in histone proteins, and methylation of cytosine residues in DNA. Non-limiting examples of suitable epigenetic modification domains include histone acetyltransferase domains, histone deacetylase domains, histone methyltransferase domains, histone demethylase domains. DNA methyltransferase domains, and DNA demethylase domains.

In embodiments in which the effector domain is a histone acetyltransferase (HAT) domain, the HAT domain can be derived from EP300 (i.e., E1A binding protein p300), CREBBP (i.e., CREB-binding protein), CDY1, CDY2, CDYL1, CLOCK, ELP3, ESA1, GCN5 (KAT2A), HAT1, KAT2B, KAT5, MYST1, MYST2, MYST3, MYST4, NCOA1, NCOA2, NCOA3, NCOAT, P/CAF, Tip60, TAFII250, or TF3C4. In embodiments wherein the effector domain is an epigenetic modification domain, the Cas12a polypeptide can be modified as discussed herein such that its endonuclease activity is eliminated. For example, the Cas12a polypeptide can be modified by mutating the RuvC-like domain such that the polypeptide no longer possesses nuclease activity.

In some embodiments, the effector domain of the fusion protein can be a transcriptional activation domain. In general, a transcriptional activation domain interacts with transcriptional control elements and/or transcriptional regulatory proteins (i.e., transcription factors, RNA polymerases, etc.) to increase and/or activate transcription of one or more genes. In some embodiments, the transcriptional activation domain can be, without limit, a herpes simplex virus VP16 activation domain, VP64 (which is a tetrameric derivative of VP16), a NFκB p65 activation domain, p53 activation domains 1 and 2, a CREB (CAMP response element binding protein) activation domain, an E2A activation domain, and an NFAT (nuclear factor of activated T-cells) activation domain. In other embodiments, the transcriptional activation domain can be Gal4, Gcn4, MLL, Rtg3, Gln3, Oaf1, Pip2, Pdr1, Pdr3, Pho4, and Leu3. The transcriptional activation domain may be wild type, or it may be a modified version of the original transcriptional activation domain. In some embodiments, the effector domain of the fusion protein is a VP16 or VP64 transcriptional activation domain. In an exemplary embodiment, the transcriptional activation domain is TV or VPR. In embodiments wherein the effector domain is a transcriptional activation domain, the Cas12a polypeptide can be modified as discussed herein such that its endonuclease activity is eliminated. For example, the Cas12a polypeptide can be modified by mutating the RuvC-like domain such that the polypeptide no longer possesses nuclease activity.

In still other embodiments, the effector domain of the fusion protein can be a transcriptional repressor domain. In general, a transcriptional repressor domain interacts with transcriptional control elements and/or transcriptional regulatory proteins (i.e., transcription factors. RNA polymerases, etc.) to decrease and/or terminate transcription of one or more genes. Non-limiting examples of suitable transcriptional repressor domains include inducible cAMP early repressor (ICER) domains. Kruppel-associated box A (KRAB-A) repressor domains. YY1 glycine rich repressor domains. Spl-like repressors, E(spl) repressors. IκB repressor, and MeCP2. In embodiments wherein the effector domain is a transcriptional repressor domain, the Cas12a polypeptide can be modified as discussed herein such that its endonuclease activity is eliminated. For example, the Cas12a polypeptide can be modified by mutating the RuvC-like domain such that the polypeptide no longer possesses nuclease activity.

In some embodiments, the effector domain of the fusion protein can be a nucleotide deaminase or a catalytic domain thereof. The nucleotide deaminase may be an adenosine deaminase or a cytidine deaminase. In general, a Cas12a fused with a deaminase domain can target a sequence in the genome of a plant through the direction of a guide RNA to perform base editing, including the introduction of C to T or A to G substitutions. In some embodiments, the adenosine deaminase can be, without limit, a member of the enzyme family known as adenosine deaminases that act on RNA (ADARs), a member of the enzyme family known as adenosine deaminases that act on tRNA (ADATs), or an adenosine deaminase domain-containing (ADAD) family member. In some embodiments, the cytidine deaminase can be, without limit, a member of the enzyme family known as apolipoprotein B mRNA-editing complex (APOBEC) family deaminase, an activation-induced deaminase (AID), or a cytidine deaminase 1 (CDA1). In embodiments wherein the effector domain is a deaminase domain, the Cas12a polypeptide can be modified as discussed herein such that its endonuclease activity is eliminated. For example, the Cas12apolypeptide can be modified by mutating the RuvC-like domain such that the polypeptide no longer possesses nuclease activity. In some embodiments, the Cas12a polypeptide has nickase activity.

In some embodiments, the effector domain of the fusion protein can be a reverse transcriptase for prime editing. Prime editing of a target sequence enables the incorporation of a nucleotide change including a single-nucleotide change (e.g., any transition or any transversion), an insertion of one or more nucleotides, or a deletion of one or more nucleotides. A Cas12a fused with a reverse transcriptase is guided to a specific DNA sequence by a modified guide RNA, named a pegRNA. The pegRNA is altered (relative to a standard guide RNA) to comprise an extended portion that provides a DNA synthesis template sequence which encodes a single strand DNA flap, which is homologous to a strand of the targeted endogenous DNA sequence to be edited, but which contains the desired one or more nucleotide changes and which, following synthesis by the reverse transcriptase, becomes incorporated into the target DNA molecule. Prime editing is disclosed in, for example, PCT Publication WO/2020/191248, the entire contents of which is hereby incorporated by reference. In embodiments wherein the effector domain is a reverse transcriptase, the Cas12a polypeptide can be modified as discussed herein such that its endonuclease activity is eliminated. For example, the Cas12a polypeptide can be modified by mutating the RuvC-like domain such that the polypeptide no longer possesses nuclease activity. In some embodiments, the Cas12a polypeptide has nickase activity.

In some embodiments, the fusion protein further comprises at least one additional domain. Non-limiting examples of suitable additional domains include nuclear localization signals, cell-penetrating or translocation domains, and marker domains.

When the effector domain of the fusion protein is a cleavage domain, a dimer comprising at least one fusion protein can form. The dimer can be a homodimer or a heterodimer. In some embodiments, the heterodimer comprises two different fusion proteins. In other embodiments, the heterodimer comprises one fusion protein and an additional protein.

The dimer can be a homodimer in which the two fusion protein monomers are identical with respect to the primary amino acid sequence. In one embodiment where the dimer is a homodimer, the Cas12a polypeptide can be modified such that the endonuclease activity is eliminated. In certain embodiments wherein the Cas12a polypeptide is modified such that endonuclease activity is eliminated, each fusion protein monomer can comprise an identical Cas12a polypeptide and an identical cleavage domain. The cleavage domain can be any cleavage domain, such as any of the exemplary cleavage domains provided herein. In such embodiments, specific guide RNAs would direct the fusion protein monomers to different but closely adjacent sites such that, upon dimer formation, the nuclease domains of the two monomers would create a double stranded break in the target DNA.

The dimer can also be a heterodimer of two different fusion proteins. For example, the Cas12a polypeptide of each fusion protein can be derived from a different Cas12a polypeptide or from an orthologous Cas12a polypeptide. For example, each fusion protein can comprise a Cas12a polypeptide derived from a different source. In these embodiments, each fusion protein would recognize a different target site (i.e., specified by the protospacer and/or PAM sequence). For example, the guide RNAs could position the heterodimer to different but closely adjacent sites such that their nuclease domains produce an effective double stranded break in the target DNA.

Alternatively, two fusion proteins of a heterodimer can have different effector domains. In embodiments in which the effector domain is a cleavage domain, each fusion protein can contain a different modified cleavage domain. In these embodiments, the Cas12a polypeptide(s) can be modified such that their endonuclease activities are eliminated. The two fusion proteins forming a heterodimer can differ in both the Cas12a polypeptide domain and the effector domain.

In any of the above-described embodiments, the homodimer or heterodimer can comprise at least one additional domain chosen from nuclear localization signals (NLSs), plastid signal peptides, mitochondrial signal peptides, signal peptides capable of trafficking proteins to multiple subcellular locations, cell-penetrating, translocation domains and marker domains, as detailed above. In any of the above-described embodiments, one or both of the Cas12a polypeptides can be modified such that endonuclease activity of the polypeptide is eliminated or modified.

The heterodimer can also comprise one fusion protein and an additional protein. For example, the additional protein can be a nuclease. In one embodiment, the nuclease is a zinc finger nuclease. A zinc finger nuclease comprises a zinc finger DNA binding domain and a cleavage domain. A zinc finger recognizes and binds three (3) nucleotides. A zinc finger DNA binding domain can comprise from about three zinc fingers to about seven zinc fingers. The zinc finger DNA binding domain can be derived from a naturally occurring protein or it can be engineered. See, for example, Beerli et al. (2002) Nat. Biotechnol. 20:135-141: Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637: Choo et al. (2000) Curr. Opin. Struct. Biol. 10:411-416; Zhang et al. (2000) J. Biol. Chem. 275 (43): 33850-33860: Doyon et al. (2008) Nat. Biotechnol. 26:702-708; and Santiago et al. (2008) Proc. Natl. Acad. Sci. USA 105:5809-5814. The cleavage domain of the zinc finger nuclease can be any cleavage domain detailed herein. In some embodiments, the zinc finger nuclease can comprise at least one additional domain chosen from nuclear localization signals, plastid signal peptides, mitochondrial signal peptides, signal peptides capable of trafficking proteins to multiple subcellular locations, cell-penetrating or translocation domains, which are detailed herein.

In certain embodiments, any of the fusion proteins detailed above or a dimer comprising at least one fusion protein may be part of a protein-RNA complex comprising at least one guide RNA. A guide RNA interacts with the Cas12a polypeptide of the fusion protein to direct the fusion protein to a specific target site, wherein the 5′ end of the guide RNA base pairs with a specific protospacer sequence.

### Nucleic Acids Encoding Cas12a Polypeptides or Fusion Proteins

Nucleic acids encoding any of the Cas12a polypeptides or fusion proteins described herein are provided. The nucleic acid can be RNA or DNA. Examples of polynucleotides that encode Cas12a polypeptides are set forth in SEQ ID NOs: 1-17. In one embodiment, the nucleic acid encoding the Cas12a polypeptide or fusion protein is mRNA. The mRNA can be 5′ capped and/or 3′ polyadenylated. In another embodiment, the nucleic acid encoding the Cas12a polypeptide or fusion protein is DNA. The DNA can be present in a vector.

Nucleic acids encoding the Cas12a polypeptide or fusion proteins can be codon optimized for efficient translation into protein in the plant cell of interest. Programs for codon optimization are available in the art (e.g., OPTIMIZER at genomes. urv.es/OPTIMIZER: OptimumGene™ from GenScript at genscript.com/codon_opt.html).

In certain embodiments, DNA encoding the Cas12a polypeptide or fusion protein can be operably linked to at least one promoter sequence. The DNA coding sequence can be operably linked to a promoter control sequence for expression in a host cell of interest. In some embodiments, the host cell is a plant cell. “Operably linked” is intended to mean a functional linkage between two or more elements. For example, an operable linkage between a promoter and a coding region of interest (e.g., region coding for a Cas12a polypeptide or guide RNA) is a functional link that allows for expression of the coding region of interest. Operably linked elements may be contiguous or non-contiguous. When used to refer to the joining of two protein coding regions, “operably linked” is intended to indicate that the coding regions are in the same reading frame.

The promoter sequence can be constitutive, regulated, growth stage-specific, or tissue-specific. It is recognized that different applications can be enhanced by the use of different promoters in the nucleic acid molecules to modulate the timing, location and/or level of expression of the Cas12a polypeptide and/or guide RNA. Such nucleic acid molecules may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

In some embodiments, the nucleic acid molecules provided herein can be combined with constitutive, tissue-preferred, developmentally-preferred or other promoters for expression in plants. Examples of constitutive promoters functional in plant cells include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. If low level expression is desired, weak promoter(s) may be used. Weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

Examples of inducible promoters are the Adhl promoter which is inducible by hypoxia or cold stress, the Hsp70) promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the ERE promoter which is estrogen induced, and the Axigl promoter which is auxin induced and tapetum specific but also active in callus (PCT US01/22169).

Examples of promoters under developmental control in plants include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding a Cas12a polypeptide and/or guide RNA comprise a cell type specific promoter. A “cell type specific” promoter is a promoter that primarily drives expression in certain cell types in one or more organs. Some examples of plant cells in which cell type specific promoters functional in plants may be primarily active include, for example, BETL cells, vascular cells in roots, leaves, stalk cells, and stem cells. The nucleic acid molecules can also include cell type preferred promoters. A “cell type preferred” promoter is a promoter that primarily drives expression mostly, but not necessarily entirely or solely in certain cell types in one or more organs. Some examples of plant cells in which cell type preferred promoters functional in plants may be preferentially active include, for example, BETL cells, vascular cells in roots, leaves, stalk cells, and stem cells. The nucleic acid molecules described herein can also comprise seed-preferred promoters. In some embodiments, the seed-preferred promoters have expression in embryo sac, early embryo, early endosperm, aleurone, and/or basal endosperm transfer cell layer (BETL).

Examples of seed-preferred promoters include, but are not limited to, 27 kD gamma zein promoter and waxy promoter, Boronat, A. et al. (1986) Plant Sci. 47:95-102: Reina, M. et al. Nucl. Acids Res. 18 (21): 6426; and Kloesgen, R. B. et al. (1986) Mol. Gen. Genet. 203:237-244. Promoters that express in the embryo, pericarp, and endosperm are disclosed in U.S. Pat. No. 6,225,529 and PCT publication WO 00/12733. The disclosures for each of these are incorporated herein by reference in their entirety.

Promoters that can drive gene expression in a plant seed-preferred manner with expression in the embryo sac, early embryo, early endosperm, aleurone and/or basal endosperm transfer cell layer (BETL) can be used in the compositions and methods disclosed herein. Such promoters include, but are not limited to, promoters that are naturally linked to Zea mays early endosperm 5 gene, Zea mays early endosperm 1 gene, Zea mays early endosperm 2 gene, GRMZM2G124663, GRMZM2G006585, GRMZM2G120008, GRMZM2G157806, GRMZM2G176390, GRMZM2G472234, GRMZM2G138727. Zea mays CLAVATAI, Zea mays MRP1, Oryza sativa PR602, Oryza sativa PR9a, Zea mays BET1, Zea mays BETL-2, Zea mays BETL-3, Zea mays BETL-4, Zea mays BETL-9, Zea mays BETL-10, Zea mays MEGI. Zea mays TCCRI, Zea mays ASPI, Oryza sativa ASPI, Triticum durum PR60, Triticum durum PR91, Triticum durum GL7, AT3G10590, AT4G18870, AT4G21080, AT5G23650, AT3G05860, AT5G42910, AT2G26320, AT3G03260, AT5G26630, AtIPT4, AtIPT8, AtLEC2, LFAH12. Additional such promoters are described in U.S. Pat. Nos. 7,803,990, 8,049,000, 7,745,697, 7,119,251, 7,964,770, 7,847,160, 7,700,836, U.S. Patent Application Publication Nos. 20100313301, 20090049571, 20090089897, 20100281569, 20100281570, 20120066795, 20040003427: PCT Publication Nos. WO/1999/050427, WO/2010/129999, WO/2009/094704, WO/2010/019996 and WO/2010/147825, each of which is herein incorporated by reference in its entirety for all purposes. Functional variants or functional fragments of the promoters described herein can also be operably linked to the nucleic acids disclosed herein.

Chemical-regulated promoters can be used to modulate the expression of a gene through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14 (2): 247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of an expression construct within a particular tissue. In certain embodiments, the tissue-preferred promoters may be active in plant tissue. Tissue-preferred promoters are known in the art. See, for example, Yamamoto et al. (1997) Plant 12 (2): 255-265; Kawamata et al. (1997) Plant Cell Physiol. 38 (7): 792-803: Hansen et al. (1997) Mol. Gen Genet. 254 (3): 337-343; Russell et al. (1997) Transgenic Res. 6 (2): 157-168: Rinehart et al. (1996) Plant Physiol. 112 (3): 1331-1341: Van Camp et al. (1996) Plant Physiol. 112 (2): 525-535: Canevascini et al. (1996) Plant Physiol. 112 (2):513-524: Yamamoto et al. (1994) Plant Cell Physiol. 35 (5):773-778; Lam (1994) Results Probl. Cell Differ. 20:181-196; Orozco et al. (1993) Plant Mol Biol. 23 (6):1129-1138: Matsuoka et al. (1993) Proc Natl. Acad. Sci. USA 90 (20):9586-9590; and Guevara-Garcia et al. (1993) Plant J. 4 (3):495-505. Such promoters can be modified, if necessary, for weak expression.

Leaf-preferred promoters are known in the art. See, for example, Yamamoto et al. (1997) Plant J. 12 (2):255-265: Kwon et al. (1994) Plant Physiol. 105:357-67; Yamamoto et al. (1994) Plant Cell Physiol. 35 (5): 773-778: Gotor et al. (1993) Plant J. 3:509-18; Orozco et al. (1993) Plant Mol. Biol. 23 (6): 1129-1138; and Matsuoka et al. (1993) Proc. Natl. Acad. Sci. USA 90 (20): 9586-9590. In addition, the promoters of cab and rubisco can also be used. See, for example. Simpson et al. (1958) EMBO J 4:2723-2729 and Timko et al. (1988) Nature 318:57-58.

Root-preferred promoters are known and can be selected from the many available from the literature or isolated de novo from various compatible species. See, for example, Hire et al. (1992) Plant Mol. Biol. 20 (2):207-218 (soy bean root-specific glutamine synthetase gene); Keller and Baumgartner (1991) Plant Cell 3 (10):1051-1061 (root-specific control element in the GRP 1.8 gene of French bean): Sanger et al. (1990) Plant Mol. Biol. 14 (3): 433-443 (root-specific promoter of the mannopine synthase (MAS) gene of Agrobacterium tumefaciens); and Miao et al. (1991) Plant Cell 3 (1): 11-22 (full-length cDNA clone encoding cytosolic glutamine synthetase (GS), which is expressed in roots and root nodules of soy bean). See also Bogusz et al. (1990) Plant Cell 2 (7): 633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a B-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and roID root-inducing genes of Agrobacterium rhizogenes (see Plant Science (Limerick) 79 (1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8 (2):343-350). The TR1′ gene, fused to nptII (neomycin phosphotransferase II) showed similar characteristics. Additional root-preferred promoters include the VIENOD-GRP3 gene promoter (Kuster et al. (1995) Plant Mol. Biol. 29 (4):759-772); and roM promoter (Capana et al. (1994) Plant Mol. Biol. 25 (4):681-691. See also U.S. Pat. Nos. 5,837,876; 5,750,386; 5,633,363; 5,459,252; 5,401,836; 5,110,732; and 5,023,179. The phaseolin gene (Murai et al. (1983) Science 23:476-482 and Sengopta-Gopalen et al. (1988) PNAS 82:3320-3324. The promoter sequence can be wild type or it can be modified for more efficient or efficacious expression.

The nucleic acid sequences encoding the Cas12a polypeptide or fusion protein can be operably linked to a promoter sequence that is recognized by a phage RNA polymerase for in vitro mRNA synthesis. In such embodiments, the in vitro-transcribed RNA can be purified for use in the methods of genome modification described herein. For example, the promoter sequence can be a T7, T3, or SP6 promoter sequence or a variation of a T7, T3, or SP6 promoter sequence. In some embodiments, the sequence encoding the Cas12a polypeptide or fusion protein can be operably linked to a promoter sequence for in vitro expression of the Cas12a polypeptide or fusion protein in plant cells. In such embodiments, the expressed protein can be purified for use in the methods of genome modification described herein.

In certain embodiments, the DNA encoding the Cas12a polypeptide or fusion protein also can be linked to a polyadenylation signal (e.g., SV40 poly A signal and other signals functional in the cells of interest) and/or at least one transcriptional termination sequence. Additionally, the sequence encoding the Cas12a polypeptide or fusion protein also can be linked to a sequence encoding at least one nuclear localization signal, at least one plastid signal peptide, at least one mitochondrial signal peptide, at least one signal peptide capable of trafficking proteins to multiple subcellular locations, at least one cell-penetrating domain, and/or at least one marker domain, described elsewhere herein.

The DNA encoding the Cas12a polypeptide or fusion protein can be present in a vector. Suitable vectors include plasmid vectors, phagemids, cosmids, artificial/mini-chromosomes, transposons, and viral vectors (e.g., lentiviral vectors, adeno-associated viral vectors, etc.). In one embodiment, the DNA encoding the Cas12a polypeptide or fusion protein is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

In some embodiments, the expression vector comprising the sequence encoding the Cas12a polypeptide or fusion protein can further comprise a sequence encoding a guide RNA. The sequence encoding the guide RNA can be operably linked to at least one transcriptional control sequence for expression of the guide RNA in the plant or plant cell of interest. For example, DNA encoding the guide RNA can be operably linked to a promoter sequence that is recognized by RNA polymerase III (Pol III). Examples of suitable Pol III promoters include, but are not limited to, mammalian U6, U3, H1, and 7SL RNA promoters and rice U6 and U3 promoters.

### Methods for Modifying a Nucleotide Sequence in a Plant Genome

Methods are provided herein for modifying a nucleotide sequence of a plant genome. Non-limiting examples of genomes include cellular, nuclear, organellar, and plasmid genomes. The methods comprise introducing into a genome host (e.g., a cell or organelle) one or more DNA-targeting polynucleotides such as a DNA-targeting RNA (“guide RNA,” “gRNA,” “CRISPR RNA.” or “crRNA”) or a DNA polynucleotide encoding a DNA-targeting RNA, wherein the DNA-targeting polynucleotide comprises: (a) a first segment comprising a nucleotide sequence that is complementary to a sequence in the target DNA; and (b) a second segment that interacts with a Cas12a polypeptide and also introducing to the genome host a Cas12a polypeptide, or a polynucleotide encoding a Cas12a polypeptide, wherein the a Cas12a polypeptide comprises: (a) a polynucleotide-binding portion that interacts with the gRNA or other DNA-targeting polynucleotide; and (b) an activity portion that exhibits site-directed enzymatic activity. The genome host can then be cultured under conditions in which the Cas12a polypeptide is expressed and cleaves the nucleotide sequence that is targeted by the gRNA. Finally, a genome host comprising the modified nucleotide sequence can be selected.

The methods disclosed herein comprise introducing into a genome host at least one Cas12a polypeptide or a nucleic acid encoding at least one Cas12a polypeptide, as described herein. In some embodiments, the Cas12a polypeptide can be introduced into the genome host as an isolated protein. In such embodiments, the Cas12a polypeptide can further comprise at least one cell-penetrating domain, which facilitates cellular uptake of the protein. In some embodiments, the Cas12a polypeptide can be introduced into the genome host as a nucleoprotein in complex with a guide polynucleotide (for instance, as a ribonucleoprotein in complex with a guide RNA). In other embodiments, the Cas12a polypeptide can be introduced into the genome host as an mRNA molecule that encodes the Cas12a polypeptide. In still other embodiments, the Cas12a polypeptide can be introduced into the genome host as a DNA molecule comprising an open reading frame that encodes the Cas12a polypeptide. In general, DNA sequences encoding the Cas12a polypeptide or fusion protein described herein are operably linked to a promoter sequence that will function in the genome host. The DNA sequence can be linear, or the DNA sequence can be part of a vector. In still other embodiments, the Cas12a polypeptide or fusion protein can be introduced into the genome host as an RNA-protein complex comprising the guide RNA or a fusion protein and the guide RNA.

In certain embodiments, mRNA encoding the Cas12a polypeptide may be targeted to an organelle (e.g., plastid or mitochondria). In certain embodiments, mRNA encoding one or more guide RNAs may be targeted to an organelle (e.g., plastid or mitochondria). In certain embodiments, mRNA encoding the Cas12a polypeptide and one or more guide RNAs may be targeted to an organelle (e.g., plastid or mitochondria). Methods for targeting mRNA to organelles are known in the art (see, e.g., U.S. Patent Application 2011/0296551; U.S. Patent Application 2011/0321187; Gómez and Pallás (2010) PLOS One 5:e12269), and are incorporated herein by reference.

In certain embodiments, DNA encoding the Cas12a polypeptide can further comprise a sequence encoding a guide RNA. In general, each of the sequences encoding the Cas12a polypeptide and the guide RNA is operably linked to one or more appropriate promoter control sequences that allow expression of the Cas12a polypeptide and the guide RNA, respectively, in the genome host. The DNA sequence encoding the Cas12a polypeptide and the guide RNA can further comprise additional expression control, regulatory, and/or processing sequence(s). The DNA sequence encoding the Cas12a polypeptide and the guide RNA can be linear or can be part of a vector.

Methods described herein further can also comprise introducing into a genome host at least one guide RNA or DNA encoding at least one polynucleotide such as a guide RNA. A guide RNA interacts with the Cas12a polypeptide to direct the Cas12a polypeptide to a specific target site, at which site the guide RNA base pairs with a specific DNA sequence in the targeted site. Guide RNAs can comprise three regions: a first region that is complementary to the target site in the targeted DNA sequence, a second region that forms a stem loop structure, and a third region that remains essentially single-stranded. The first region of each guide RNA is different such that each guide RNA guides a Cas12a polypeptide to a specific target site. The second and third regions of each guide RNA can be the same in all guide RNAs.

One region of the guide RNA is complementary to a sequence (i.e., protospacer sequence) at the target site in the targeted DNA such that the first region of the guide RNA can base pair with the target site. In various embodiments, the first region of the guide RNA can comprise from about 8 nucleotides to more than about 30 nucleotides. For example, the region of base pairing between the first region of the guide RNA and the target site in the nucleotide sequence can be about 8, about 9, about 10, about 11, about 12, about 13, about 14, about 15, about 16, about 17, about 18, about 19, about 20, about 22, about 23, about 24, about 25, about 27, about 30 or more than 30 nucleotides in length. In an exemplary embodiment, the first region of the guide RNA is about 23, 24, or 25 nucleotides in length. The guide RNA also can comprise a second region that forms a secondary structure. In some embodiments, the secondary structure comprises a stem or hairpin. The length of the stem can vary. For example, the stem can range from about 5, to about 6, to about 10, to about 15, to about 20, to about 25 base pairs in length. The stem can comprise one or more bulges of 1 to about 10 nucleotides. The overall length of the second region can range from about 14 to about 25 nucleotides in length. In certain embodiments, the loop is about 3, 4, or 5 nucleotides in length and the stem comprises about 5, 6, 7, 8, 9, or 10 base pairs.

The guide RNA can also comprise a third region that remains essentially single-stranded. Thus, the third region has no complementarity to any nucleotide sequence in the cell of interest and has no complementarity to the rest of the guide RNA. The length of the third region can vary. In general, the third region is more than about 4 nucleotides in length. For example, the length of the third region can range from about 5 to about 60 nucleotides in length. The combined length of the second and third regions (also called the universal or scaffold region) of the guide RNA can range from about 30 to about 120 nucleotides in length. In one aspect, the combined length of the second and third regions of the guide RNA range from about 40 to about 45 nucleotides in length.

In some embodiments, the guide RNA comprises a single molecule comprising all three regions. In other embodiments, the guide RNA can comprise two separate molecules. The first RNA molecule can comprise the first region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 25 nucleotides) that base pairs to the other sequence to form a functional guide RNA.

In certain embodiments, the guide RNA can be introduced into the genome host as an RNA molecule. The RNA molecule can be transcribed in vitro. Alternatively, the RNA molecule can be chemically synthesized. In other embodiments, the guide RNA can be introduced into the genome host as a DNA molecule. In such cases, the DNA encoding the guide RNA can be operably linked to one or more promoter sequences for expression of the guide RNA in the genome host. For example, the RNA coding sequence can be operably linked to a promoter sequence that is recognized by RNA polymerase III (Pol III).

The DNA molecule encoding the guide RNA can be linear or circular. In some embodiments, the DNA sequence encoding the guide RNA can be part of a vector. Suitable vectors include plasmid vectors, phagemids, cosmids, artificial/mini-chromosomes, transposons, and viral vectors. In an exemplary embodiment, the DNA encoding the guide RNA is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences. Kozak sequences, polyadenylation sequences, transcriptional termination sequences, etc.), selectable marker sequences (e.g., antibiotic resistance genes), origins of replication, and the like.

In embodiments in which both the Cas12a polypeptide and the guide RNA are introduced into the genome host as DNA molecules, each can be part of a separate molecule (e.g., one vector containing Cas12a polypeptide or fusion protein coding sequence and a second vector containing guide RNA coding sequence) or both can be part of the same molecule (e.g., one vector containing coding (and regulatory) sequence for both the Cas12a polypeptide or fusion protein and the guide RNA).

Cas12a proteins are RNA guided nucleases. Its cleavage relies on a guide RNA comprising a guide sequence, where the guide sequence hybridizes with the target nucleotide sequence to form a DNA/RNA heteroduplex. A Cas12a polypeptide in conjunction with a guide RNA is directed to a target site in a genome host, wherein the Cas12a polypeptide introduces a double-stranded break in the targeted DNA. The target site has no sequence limitation except that the sequence is immediately preceded (upstream) by a consensus sequence. This consensus sequence is also known as a protospacer adjacent motif (PAM). It is well-known in the art that a suitable PAM sequence must be located at the correct location relative to the targeted DNA sequence to allow the Cas12a nuclease to produce the desired double-stranded break. Cas12a PAM sequences are T-rich sequences. In some embodiments, the PAM sequence is 5′ TTTV 3′ or 5′ VTTV 3′, wherein V is A, C, or G. As detailed herein, the first region of the guide RNA is complementary to the protospacer of the target sequence. Typically, the first region of the guide RNA is about 19 to 21 nucleotides in length.

The target site can be in the coding region of a gene, in an intron of a gene, in a control region of a gene, in a non-coding region between genes, etc. The gene can be a protein coding gene or an RNA coding gene. The gene can be any gene of interest as described herein.

In some embodiments, the methods disclosed herein further comprise introducing at least one donor polynucleotide into a genome host. A donor polynucleotide comprises at least one donor sequence. In some aspects, a donor sequence of the donor polynucleotide corresponds to an endogenous or native sequence found in the targeted DNA. For example, the donor sequence can be essentially identical to a portion of the DNA sequence at or near the targeted site, but which comprises at least one nucleotide change. Thus, the donor sequence can comprise a modified version of the wild type sequence at the targeted site such that, upon integration or exchange with the native sequence, the sequence at the targeted location comprises at least one nucleotide change. For example, the change can be an insertion of one or more nucleotides, a deletion of one or more nucleotides, a substitution of one or more nucleotides, or combinations thereof. As a consequence of the integration of the modified sequence, the genome host can produce a modified gene product from the targeted chromosomal sequence.

The donor sequence of the donor polynucleotide can alternatively correspond to an exogenous sequence. As used herein, an “exogenous” sequence refers to a sequence that is not native to the genome host, or a sequence whose native location in the genome host is in a different location. For example, the exogenous sequence can comprise a protein coding sequence, which can be operably linked to an exogenous promoter control sequence such that, upon integration into the genome, the genome host is able to express the protein coded by the integrated sequence. For example, the donor sequence can be any gene of interest, such as those encoding agronomically important traits as described elsewhere herein. Alternatively, the exogenous sequence can be integrated into the targeted DNA sequence such that its expression is regulated by an endogenous promoter control sequence. In other iterations, the exogenous sequence can be a transcriptional control sequence, another expression control sequence, or an RNA coding sequence. Integration of an exogenous sequence into a targeted DNA sequence is termed a “knock in.” The donor sequence can vary in length from several nucleotides to hundreds of nucleotides to hundreds of thousands of nucleotides.

In some embodiments, the donor sequence in the donor polynucleotide is flanked by an upstream sequence and a downstream sequence, which have substantial sequence identity to sequences located upstream and downstream, respectively, of the targeted site. Because of these sequence similarities, the upstream and downstream sequences of the donor polynucleotide permit homologous recombination between the donor polynucleotide and the targeted sequence such that the donor sequence can be integrated into (or exchanged with) the targeted DNA sequence.

The upstream sequence, as used herein, refers to a nucleic acid sequence that shares substantial sequence identity with a DNA sequence upstream of the targeted site. Similarly, the downstream sequence refers to a nucleic acid sequence that shares substantial sequence identity with a DNA sequence downstream of the targeted site. As used herein, the phrase “substantial sequence identity” refers to sequences having at least about 75% sequence identity. Thus, the upstream and downstream sequences in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with sequence upstream or downstream to the targeted site. In an exemplary embodiment, the upstream and downstream sequences in the donor polynucleotide can have about 95% or 100% sequence identity with nucleotide sequences upstream or downstream to the targeted site. In one embodiment, the upstream sequence shares substantial sequence identity with a nucleotide sequence located immediately upstream of the targeted site (i.e., adjacent to the targeted site). In other embodiments, the upstream sequence shares substantial sequence identity with a nucleotide sequence that is located within about one hundred (100) nucleotides upstream from the targeted site. Thus, for example, the upstream sequence can share substantial sequence identity with a nucleotide sequence that is located about 1 to about 20, about 21 to about 40, about 41 to about 60, about 61 to about 80, or about 81 to about 100 nucleotides upstream from the targeted site. In one embodiment, the downstream sequence shares substantial sequence identity with a nucleotide sequence located immediately downstream of the targeted site (i.e., adjacent to the targeted site). In other embodiments, the downstream sequence shares substantial sequence identity with a nucleotide sequence that is located within about one hundred (100) nucleotides downstream from the targeted site. Thus, for example, the downstream sequence can share substantial sequence identity with a nucleotide sequence that is located about 1 to about 20, about 21 to about 40, about 41 to about 60, about 61 to about 80, or about 81 to about 100 nucleotides downstream from the targeted site.

Each upstream or downstream sequence can range in length from about 20 nucleotides to about 5000 nucleotides. In some embodiments, upstream and downstream sequences can comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 nucleotides. In exemplary embodiments, upstream and downstream sequences can range in length from about 50 to about 1500 nucleotides.

Donor polynucleotides comprising the upstream and downstream sequences with sequence similarity to the targeted nucleotide sequence can be linear or circular. In embodiments in which the donor polynucleotide is circular, it can be part of a vector. For example, the vector can be a plasmid vector.

In certain embodiments, the donor polynucleotide can additionally comprise at least one targeted cleavage site that is recognized by the Cas12a polypeptide. The targeted cleavage site added to the donor polynucleotide can be placed upstream or downstream or both upstream and downstream of the donor sequence. For example, the donor sequence can be flanked by targeted cleavage sites such that, upon cleavage by the Cas12a polypeptide, the donor sequence is flanked by overhangs that are compatible with those in the nucleotide sequence generated upon cleavage by the Cas12a polypeptide. Accordingly, the donor sequence can be ligated with the cleaved nucleotide sequence during repair of the double stranded break by a non-homologous repair process. Generally, donor polynucleotides comprising the targeted cleavage site(s) will be circular (e.g., can be part of a plasmid vector).

The donor polynucleotide can be a linear molecule comprising a short donor sequence with optional short overhangs that are compatible with the overhangs generated by the Cas12a polypeptide. In such embodiments, the donor sequence can be ligated directly with the cleaved chromosomal sequence during repair of the double-stranded break. In some instances, the donor sequence can be less than about 1,000, less than about 500, less than about 250, or less than about 100 nucleotides. In certain cases, the donor polynucleotide can be a linear molecule comprising a short donor sequence with blunt ends. In other iterations, the donor polynucleotide can be a linear molecule comprising a short donor sequence with 5′ and/or 3′ overhangs. The overhangs can comprise 1, 2, 3, 4, or 5 nucleotides.

In some embodiments, the donor polynucleotide will be DNA. The DNA may be single-stranded or double-stranded and/or linear or circular. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. In certain embodiments, the donor polynucleotide comprising the donor sequence can be part of a plasmid vector. In any of these situations, the donor polynucleotide comprising the donor sequence can further comprise at least one additional sequence.

In some embodiments, the method can comprise introducing one Cas12a polypeptide (or encoding nucleic acid) and one guide RNA (or encoding DNA) into a genome host, wherein the Cas12a polypeptide introduces one double-stranded break in the targeted DNA. In embodiments in which an optional donor polynucleotide is not present, the double-stranded break in the nucleotide sequence can be repaired by a non-homologous end-joining (NHEJ) repair process. Because NHEJ is error-prone, deletions of at least one nucleotide, insertions of at least one nucleotide, substitutions of at least one nucleotide, or combinations thereof can occur during the repair of the break. Accordingly, the targeted nucleotide sequence can be modified or inactivated. For example, a single nucleotide change (SNP) can give rise to an altered protein product, or a shift in the reading frame of a coding sequence can inactivate or “knock out” the sequence such that no protein product is made. In embodiments in which the optional donor polynucleotide is present, the donor sequence in the donor polynucleotide can be exchanged with or integrated into the nucleotide sequence at the targeted site during repair of the double-stranded break. For example, in embodiments in which the donor sequence is flanked by upstream and downstream sequences having substantial sequence identity with upstream and downstream sequences, respectively, of the targeted site in the nucleotide sequence, the donor sequence can be exchanged with or integrated into the nucleotide sequence at the targeted site during repair mediated by homology-directed repair process. Alternatively, in embodiments in which the donor sequence is flanked by compatible overhangs (or the compatible overhangs are generated in situ by the Cas12a polypeptide) the donor sequence can be ligated directly with the cleaved nucleotide sequence by a non-homologous repair process during repair of the double-stranded break. Exchange or integration of the donor sequence into the nucleotide sequence modifies the targeted nucleotide sequence or introduces an exogenous sequence into the targeted nucleotide sequence.

The methods disclosed herein can also comprise introducing one or more Cas12a polypeptides (or encoding nucleic acids) and two guide polynucleotides (or encoding DNAs) into a genome host, wherein the Cas12a polypeptides introduce two double-stranded breaks in the targeted nucleotide sequence. The two breaks can be within several base pairs, within tens of base pairs, or can be separated by many thousands of base pairs. In embodiments in which an optional donor polynucleotide is not present, the resultant double-stranded breaks can be repaired by a non-homologous repair process such that the sequence between the two cleavage sites is lost and/or deletions of at least one nucleotide, insertions of at least one nucleotide, substitutions of at least one nucleotide, or combinations thereof can occur during the repair of the break(s). In embodiments in which an optional donor polynucleotide is present, the donor sequence in the donor polynucleotide can be exchanged with or integrated into the targeted nucleotide sequence during repair of the double-stranded breaks by either a homology-based repair process (e.g., in embodiments in which the donor sequence is flanked by upstream and downstream sequences having substantial sequence identity with upstream and downstream sequences, respectively, of the targeted sites in the nucleotide sequence) or a non-homologous repair process (e.g., in embodiments in which the donor sequence is flanked by compatible overhangs).

Plant cells possess nuclear, plastid, and mitochondrial genomes. The compositions and methods of the present disclosure may be used to modify the sequence of the nuclear, plastid, and/or mitochondrial genome, or may be used to modulate the expression of a gene or genes encoded by the nuclear, plastid, and/or mitochondrial genome. Accordingly, “chromosome” or “chromosomal” is generally refers to the nuclear, plastid, or mitochondrial genomic DNA. “Genome” as it applies to plant cells encompasses not only chromosomal DNA found within the nucleus, but organelle DNA found within subcellular components (e.g., mitochondria or plastids) of the cell. Any nucleotide sequence of interest in a plant cell, organelle, or embryo can be modified using the methods described herein. In specific embodiments, the methods disclosed herein are used to modify a nucleotide sequence encoding an agronomically important trait, such as a plant hormone, plant defense protein, a nutrient transport protein, a biotic association protein, a desirable input trait, a desirable output trait, a stress resistance gene, a disease/pathogen resistance gene, a male sterility, a developmental gene, a regulatory gene, a gene involved in photosynthesis, a DNA repair gene, a transcriptional regulatory gene or any other polynucleotide and/or polypeptide of interest. Agronomically important traits such as oil, starch, and protein content can also be modified. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

The Cas12a polypeptide (or encoding nucleic acid), the guide RNA(s) (or encoding DNA), and the optional donor polynucleotide(s) can be introduced into a plant cell, organelle, or plant embryo by a variety of means, including transformation. Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio Technology 6:923-926 (soy bean); Finer and McMullen (1991) In Vitro Cell Dev. Biol. 27P:175-182 (soy bean); Singh et al. (1998) Theor. Appl. Genet. 96:319-324 (soy bean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855:5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae): De Wet et al. (1985) in The Experimental Manipulation of Ovule Tissues, ed. Chapman et al. (Longman, New York), pp. 197-209 (pollen): Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750) (maize via Agrobacterium tumefaciens); all of which are herein incorporated by reference. Site-specific genome editing of plant cells by biolistic introduction of a ribonucleoprotein comprising a nuclease and suitable guide RNA has been demonstrated (Svitashev et al (2016) Nat Commun 7:13274); these methods are herein incorporated by reference. “Stable transformation” is intended to mean that the nucleotide construct introduced into a plant integrates into the genome of the plant and is capable of being inherited by the progeny thereof. The nucleotide construct may be integrated into the nuclear, plastid, or mitochondrial genome of the plant. Methods for plastid transformation are known in the art (see, e.g., Chloroplast Biotechnology: Methods and Protocols (2014) Pal Maliga, ed. and U.S. Patent Application 2011/0321187), and methods for plant mitochondrial transformation have been described in the art (see, e.g., U.S. Patent Application 2011/0296551), herein incorporated by reference.

The cells that have been transformed may be grown into plants (i.e., cultured) in accordance with conventional ways. See, for example, McCormick et al. (1986) Plant Cell Reports 5:81-84. In this manner, the present disclosure provides transformed seed having a nucleic acid modification stably incorporated into their genome.

“Introduced” in the context of inserting a nucleic acid fragment (e.g., a recombinant DNA construct) into a cell, means “transfection” or “transformation” or “transduction” and includes reference to the incorporation of a nucleic acid fragment into a plant cell where the nucleic acid fragment may be incorporated into the genome of the cell (e.g., nuclear chromosome, plasmid, plastid chromosome or mitochondrial chromosome), converted into an autonomous replicon, or transiently expressed (e.g., transfected mRNA).

The methods and compositions of the present disclosure may be used for transformation of any plant species, including, but not limited to, monocots and dicots (i.e., monocotyledonous and dicotyledonous, respectively). Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soy bean (Glycine max), tobacco (Nicotiana tabacum), potato (Solanum tuberosum), peanuts (Arachis hypogaea), cotton (Gossypium barbadense. Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), eucalyptus (Eucalyptus spp.), oats (Avena sativa), barley (Hordeum vulgare), vegetables, ornamentals, and conifers. In certain embodiments, the plant is rice (Oryza sativa), tomato (Solanum lycopersicum), or poplar (Populus spp.).

The Cas12a polypeptides (or encoding nucleic acid), the guide RNA(s) (or DNAs encoding the guide RNA), and the optional donor polynucleotide(s) can be introduced into the plant cell, organelle, or plant embryo simultaneously or sequentially. The ratio of the Cas12a polypeptides (or encoding nucleic acid) to the guide RNA(s) (or encoding DNA) generally will be about stoichiometric such that the two components can form an RNA-protein complex with the target DNA. In one embodiment, DNA encoding a Cas12a polypeptide and DNA encoding a guide RNA are delivered together within the plasmid vector.

A “subject plant or plant cell” is one in which genetic alteration, such as a mutation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell. Thus, the expression levels are higher or lower than those in the control plant depending on the methods of the disclosure.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

While the present disclosure is described in terms of transformed plants, it is recognized that transformed organisms of the disclosure also include plant cells, plant protoplasts, plant cell tissue cultures from which plants can be regenerated, plant calli, plant clumps, and plant cells that are intact in plants or parts of plants such as embryos, pollen, ovules, flowers, glumes, panicles, leaves, stems, roots, root tips, anthers, pistils and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

The methods disclosed herein can be used for insertion of heterologous genes and/or modification of native plant gene expression to achieve desirable plant traits. Such traits include, for example, disease resistance, herbicide tolerance, drought tolerance, salt tolerance, insect resistance, resistance against parasitic weeds, improved plant nutritional value, improved forage digestibility, increased grain yield, cytoplasmic male sterility, altered fruit ripening, increased storage life of plants or plant parts, reduced allergen production, and increased or decreased lignin content. Genes capable of conferring these desirable traits are disclosed in U.S. Patent Application 2016/0208243, herein incorporated by reference.

Derivatives of coding sequences can be made using the methods disclosed herein to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

The methods disclosed herein can be used to modify herbicide resistance traits including genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the GAT gene: see, for example, U.S. Publication No. 20040082770 and WO 03/092360); or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron. Additional herbicide resistance traits are described for example in U.S. Patent Application 2016/0208243, herein incorporated by reference.

Sterility genes can also be modified and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development. Additional sterility traits are described for example in U.S. Patent Application 2016/0208243, herein incorporated by reference.

The quality of grain can be altered by modifying genes encoding traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be altered by modifying a gene or that could increase for example, starch for ethanol production, or provide expression of proteins. Another important commercial use of modified plants is the production of polymers and bioplastics such as described in U.S. Pat. No. 5,602,321. Genes such as B-Ketothiolase, PHBase (polyhydroxy butyrate synthase), and acetoacetyl-CoA reductase (see Schubert et al. (1988).J. Bacteriol. 170:5837-5847) facilitate expression of polyhydroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

### Methods for Modulating Gene Expression

The methods disclosed herein further encompass modification of a nucleotide sequence or regulating expression of a nucleotide sequence in a genome host. The methods can comprise introducing into the genome host at least one fusion protein or nucleic acid encoding at least one fusion protein, wherein the fusion protein comprises a Cas12a polypeptide or a fragment or variant thereof and an effector domain, and (b) at least one guide RNA or DNA encoding the guide RNA, wherein the guide RNA guides the Cas12a polypeptide of the fusion protein to a target site in the targeted DNA and the effector domain of the fusion protein modifies the chromosomal sequence or regulates expression of one or more genes in near the targeted DNA sequence.

Fusion proteins comprising a Cas12a polypeptide or a fragment or variant thereof and an effector domain are described herein. In general, the fusion proteins disclosed herein can further comprise at least one nuclear localization signal, plastid signal peptide, mitochondrial signal peptide, or signal peptide capable of trafficking proteins to multiple subcellular locations. Nucleic acids encoding fusion proteins are described herein. In some embodiments, the fusion protein can be introduced into the genome host as an isolated protein (which can further comprise a cell-penetrating domain). Furthermore, the isolated fusion protein can be part of a protein-RNA complex comprising the guide RNA. In other embodiments, the fusion protein can be introduced into the genome host as a RNA molecule (which can be capped and/or polyadenylated). In still other embodiments, the fusion protein can be introduced into the genome host as a DNA molecule. For example, the fusion protein and the guide RNA can be introduced into the genome host as discrete DNA molecules or as part of the same DNA molecule. Such DNA molecules can be plasmid vectors.

In some embodiments, the method further comprises introducing into the genome host at least one donor polynucleotide as described elsewhere herein. Means for introducing molecules into genome hosts such as cells, as well as means for culturing cells (including cells comprising organelles) are described herein.

In certain embodiments in which the effector domain of the fusion protein is a cleavage domain, the method can comprise introducing into the genome host one fusion protein (or nucleic acid encoding one fusion protein) and two guide RNAs (or DNA encoding two guide RNAs). The two guide RNAs direct the fusion protein to two different target sites in the chromosomal sequence, wherein the fusion protein dimerizes (e.g., forms a homodimer) such that the two cleavage domains can introduce a double stranded break into the targeted DNA sequence. In embodiments in which the optional donor polynucleotide is not present, the double-stranded break in the targeted DNA sequence can be repaired by a non-homologous end-joining (NHEJ) repair process. Because NHEJ is error-prone, deletions of at least one nucleotide. insertions of at least one nucleotide, substitutions of at least one nucleotide, or combinations thereof can occur during the repair of the break. Accordingly, the targeted chromosomal sequence can be modified or inactivated. For example, a single nucleotide change (SNP) can give rise to an altered protein product, or a shift in the reading frame of a coding sequence can inactivate or “knock out” the sequence such that no protein product is made. In embodiments in which the optional donor polynucleotide is present, the donor sequence in the donor polynucleotide can be exchanged with or integrated into the targeted DNA sequence at the targeted site during repair of the double-stranded break. For example, in embodiments in which the donor sequence is flanked by upstream and downstream sequences having substantial sequence identity with upstream and downstream sequences, respectively, of the targeted site in the targeted DNA sequence, the donor sequence can be exchanged with or integrated into the targeted DNA sequence at the targeted site during repair mediated by homology-directed repair process. Alternatively, in embodiments in which the donor sequence is flanked by compatible overhangs (or the compatible overhangs are generated in situ by the Cas12a polypeptide) the donor sequence can be ligated directly with the cleaved targeted DNA sequence by a non-homologous repair process during repair of the double-stranded break. Exchange or integration of the donor sequence into the targeted DNA sequence modifies the targeted DNA sequence or introduces an exogenous sequence into the targeted DNA sequence.

In other embodiments in which the effector domain of the fusion protein is a cleavage domain, the method can comprise introducing into the genome host two different fusion proteins (or nucleic acid encoding two different fusion proteins) and two guide RNAs (or DNA encoding two guide RNAs). The fusion proteins can differ as detailed elsewhere herein. Each guide RNA directs a fusion protein to a specific target site in the targeted DNA sequence, wherein the fusion proteins can dimerize (e.g., form a heterodimer) such that the two cleavage domains can introduce a double stranded break into the targeted DNA sequence. In embodiments in which the optional donor polynucleotide is not present, the resultant double-stranded breaks can be repaired by a non-homologous repair process such that deletions of at least one nucleotide, insertions of at least one nucleotide, substitutions of at least one nucleotide, or combinations thereof can occur during the repair of the break. In embodiments in which the optional donor polynucleotide is present, the donor sequence in the donor polynucleotide can be exchanged with or integrated into the chromosomal sequence during repair of the double-stranded break by either a homology-based repair process (e.g., in embodiments in which the donor sequence is flanked by upstream and downstream sequences having substantial sequence identity with upstream and downstream sequences, respectively, of the targeted sites in the chromosomal sequence) or a non-homologous repair process (e.g., in embodiments in which the donor sequence is flanked by compatible overhangs).

In certain embodiments in which the effector domain of the fusion protein is a transcriptional activation domain or a transcriptional repressor domain, the method can comprise introducing into the genome host one fusion protein (or nucleic acid encoding one fusion protein) and one guide RNA (or DNA encoding one guide RNA). The guide RNA directs the fusion protein to a specific targeted DNA sequence, wherein the transcriptional activation domain or a transcriptional repressor domain activates or represses expression, respectively, of a gene or genes located near the targeted DNA sequence. That is, transcription may be affected for genes in close proximity to the targeted DNA sequence or may be affected for genes located at further distance from the targeted DNA sequence. It is well-known in the art that gene transcription can be regulated by distantly located sequences that may be located thousands of bases away from the transcription start site or even on a separate chromosome (Harmston and Lenhard (2013) Nucleic Acids Res 41:7185-7199).

In alternative embodiments in which the effector domain of the fusion protein is an epigenetic modification domain, the method can comprise introducing into the genome host one fusion protein (or nucleic acid encoding one fusion protein) and one guide RNA (or DNA encoding one guide RNA). The guide RNA directs the fusion protein to a specific targeted DNA sequence, wherein the epigenetic modification domain modifies the structure of the targeted DNA sequence. Epigenetic modifications include acetylation, methylation of histone proteins and/or nucleotide methylation. In some instances, structural modification of the chromosomal sequence leads to changes in expression of the chromosomal sequence.

### Plants Comprising a Genetic Modification

Provided herein are plants, plant cells, plant organelles, and plant embryos comprising at least one nucleotide sequence that has been modified using a Cas12a polypeptide-mediated or fusion protein-mediated process as described herein. Also provided are plants, plant cells, plant organelles, and plant embryos comprising at least one DNA or RNA molecule encoding Cas12a polypeptide or fusion protein targeted to a chromosomal sequence of interest or a fusion protein, at least one guide RNA, and optionally one or more donor polynucleotide(s). The genetically modified plants disclosed herein can be heterozygous for the modified nucleotide sequence or homozygous for the modified nucleotide sequence. Plant cells comprising one or more genetic modifications in organellar DNA may be heteroplasmic or homoplasmic.

The modified chromosomal sequence of the plants, plant cells, plant organelles, and plant embryos may be modified such that it is inactivated, has up-regulated or down-regulated expression, or produces an altered protein product, or comprises an integrated sequence. The modified chromosomal sequence may be inactivated such that the sequence is not transcribed and/or a functional protein product is not produced. Thus, a genetically modified plant comprising an inactivated chromosomal sequence may be termed a “knock out” or a “conditional knock out.” The inactivated chromosomal sequence can include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional protein is not produced. The inactivated chromosomal sequence comprises no exogenously introduced sequence. Also included herein are genetically modified plants in which two, three, four, five, six, seven, eight, nine, or ten or more chromosomal sequences are inactivated.

The modified chromosomal sequence can also be altered such that it codes for a variant protein product. For example, a genetically modified plant comprising a modified chromosomal sequence can comprise a targeted point mutation(s) or other modification such that an altered protein product is produced. In one embodiment, the chromosomal sequence can be modified such that at least one nucleotide is changed and the expressed protein comprises one changed amino acid residue (missense mutation). In another embodiment, the chromosomal sequence can be modified to comprise more than one missense mutation such that more than one amino acid is changed. Additionally, the chromosomal sequence can be modified to have a three nucleotide deletion or insertion such that the expressed protein comprises a single amino acid deletion or insertion. The altered or variant protein can have altered properties or activities compared to the wild type protein, such as altered substrate specificity, altered enzyme activity, altered kinetic rates, etc.

In some embodiments, the genetically modified plant can comprise at least one chromosomally integrated nucleotide sequence. A genetically modified eukaryote comprising an integrated sequence may be termed a “knock in” or a “conditional knock in.” The nucleotide sequence that is an integrated sequence can, for example, encode an orthologous protein, an endogenous protein, or combinations of both. In one embodiment, a sequence encoding an orthologous protein or an endogenous protein can be integrated into a nuclear or organellar chromosomal sequence encoding a protein such that the chromosomal sequence is inactivated, but the exogenous sequence is expressed. In such a case, the sequence encoding the orthologous protein or endogenous protein may be operably linked to a promoter control sequence. Alternatively, a sequence encoding an orthologous protein or an endogenous protein may be integrated into a nuclear or organellar chromosomal sequence without affecting expression of a chromosomal sequence. The present disclosure also encompasses genetically modified plants in which two, three, four, five, six, seven, eight, nine, or ten or more sequences, including sequences encoding protein(s), are integrated into the genome. Any gene of interest as disclosed herein can be introduced and/or integrated into the chromosomal sequence of the plant nucleus or organelle. In particular embodiments, genes that increase plant growth or yield are integrated into the chromosome. The chromosomally integrated sequence encoding a protein can encode the wild type form of a protein of interest or can encode a protein comprising at least one modification such that an altered version of the protein is produced.

In certain embodiments, the genetically modified plant can comprise at least one modified chromosomal sequence encoding a protein such that the expression pattern of the protein is altered. For example, regulatory regions controlling the expression of the protein, such as a promoter or a transcription factor binding site, can be altered such that the protein is over-expressed, or the tissue-specific or temporal expression of the protein is altered, or a combination thereof. Alternatively, the expression pattern of the protein can be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art.

The following numbered embodiments also form part of the present disclosure:
1. A method of modifying a nucleotide sequence at a target site in the genome of a plant cell, the method comprising: introducing into the plant cell (i) a Cas12a polypeptide, or a polynucleotide encoding the Cas12a polypeptide, wherein the Cas12a polypeptide has at least 80%, at least 90%, at least 95%, at least 98%, or at least 99% sequence identity to the amino acid sequence set forth in any of SEQ ID NOs: 18-34; and (ii) a DNA-targeting RNA, or a DNA polynucleotide encoding a DNA-targeting RNA, wherein the DNA-targeting RNA is capable of forming a complex with the Cas12a polypeptide and directing the complex to the target site.
2. The method of embodiment 1, wherein the polynucleotide encoding the Cas12a polypeptide has at least 80%, at least 90%, at least 95%, at least 98%, or at least 99% sequence identity to the nucleotide sequence set forth in any of SEQ ID NOs: 1-17.
3. The method of embodiment 1 or embodiment 2, wherein the Cas12a polypeptide comprises the amino acid sequence set forth in any of SEQ ID NOs: 18-34.
4. The method of any one of embodiments 1-3, wherein the polynucleotide encoding the Cas12a polypeptide comprises the nucleotide sequence set forth in any of SEQ ID NOs: 1-17.
5. The method of any one of embodiments 1-4, wherein the Cas12a polypeptide introduces a double-strand break (DSB) at the target site to produce the modified nucleotide sequence.
6. The method of embodiment 5, wherein the DSB at the target site comprises a staggered DSB.
7. The method of any one of embodiments 1-6, wherein the modified nucleotide sequence comprises insertion of heterologous DNA into the genome of the plant cell, deletion of a nucleotide sequence from the genome of the plant cell, or mutation of at least one nucleotide in the genome of the plant cell.
8. The method of any one of embodiments 1-7, wherein the plant cell is from a monocotyledonous or a dicotyledonous species.
9. The method of any one of embodiments 1-8, wherein the plant cell is from Oryza sativa. Solanum lycopersicum, or Populus sp.
10. The method of any one of embodiments 1-9, wherein expression of the Cas12a polypeptide is under the control of an inducible promoter, a constitutive promoter, a cell type-specific promoter, or a developmentally preferred promoter.
11. The method of any one of embodiments 1-10, wherein the DNA-targeting RNA comprises: (a) a first segment comprising a nucleotide sequence that is complementary to a sequence in the target DNA; and (b) a second segment that interacts with the Cas12a polypeptide.
12. The method of any one of embodiments 1-11, wherein the target site is located immediately 3′ of a PAM site in the genome of the plant cell.
13. The method of embodiment 12, wherein the PAM site comprises TTTV.
14. The method of any one of embodiments 1-13, wherein the introducing is at a temperature from about 22° C. to about 32° C. or about 22° C. to about 25° C., optionally wherein the temperature is about 22° C. or about 25° C.
15. The method of any one of embodiments 1-14, wherein the polynucleotide sequence encoding the Cas12a polypeptide is codon-optimized for expression in a plant cell.
16. The method of any one of embodiments 1-15, wherein the Cas12a polypeptide comprises one or more mutations that reduce or eliminate the nuclease activity of the Cas12a poly peptide.
17. The method of any one of embodiments 1-16, wherein the Cas12a polypeptide has nickase activity.
18. The method of any one of embodiments 1-17, wherein the Cas12a polypeptide is fused to a deaminase domain, and wherein the modified nucleotide sequence comprises a base edit at the target site.
19. The method of any one of embodiments 1-8, wherein the Cas12a polypeptide is fused to a reverse transcriptase domain, and wherein the DNA-targeting RNA is a prime editing guide RNA (pegRNA).
20. The method of any one of embodiments 1-19, wherein polynucleotide encoding the Cas12a polypeptide is present in a vector.
21. The method of any one of embodiments 1-20, further comprising regenerating a plant from the plant cell.
22. A plant cell produced by the method of any one of embodiments 1-20.
23. A plant comprising the plant cell of embodiment 22.
24. A method of modulating the expression of a target gene in a plant cell, the method comprising: introducing into the plant cell (i) a Cas12a polypeptide, or a polynucleotide encoding the Cas12a polypeptide, wherein the Cas12a polypeptide has at least 80%, at least 90%, at least 95%, at least 98%, or at least 99% sequence identity to the amino acid sequence set forth in any of SEQ ID NOs: 18-34, and wherein the Cas12a polypeptide comprises one or more mutations that reduce or eliminate the nuclease activity of the Cas12a polypeptide; and (ii) a DNA-targeting RNA, or a DNA polynucleotide encoding a DNA-targeting RNA, wherein the DNA-targeting RNA is capable of forming a complex with the Cas12a polypeptide and directing the complex to the target site.
25. The method of embodiment 24, wherein the polynucleotide encoding the Cas12a polypeptide has at least 80%, at least 90%, at least 95%, at least 98%, or at least 99% sequence identity to the nucleotide sequence set forth in any of SEQ ID NOs: 1-17.
26. The method of embodiment 24 or embodiment 25, wherein the Cas12a polypeptide comprises the amino acid sequence set forth in any of SEQ ID NOs: 18-34.
27. The method of any one of embodiments 24-26, wherein the polynucleotide encoding the Cas12a polypeptide comprises the nucleotide sequence set forth in any of SEQ ID NOs: 1-17.
28. The method of any one of embodiments 24-27, wherein the target gene is upregulated or downregulated.
29. The method of any one of embodiments 24-28, wherein the Cas12a polypeptide is fused to a transcriptional activation domain or a transcriptional repression domain.
30. The method of any one of embodiments 24-29, wherein the plant cell is from a monocotyledonous or a dicotyledonous species.
31. The method of any one of embodiments 24-30, wherein the plant cell is from Oryza sativa. Solanum lycopersicum, or Populus sp.
32. The method of any one of embodiments 24-31, wherein expression of the Cas12a polypeptide is under the control of an inducible promoter, a constitutive promoter, a cell type-specific promoter, or a developmentally preferred promoter.
33. The method of any one of embodiments 24-32, wherein the DNA-targeting RNA comprises: (a) a first segment comprising a nucleotide sequence that is complementary to a sequence in the target DNA; and (b) a second segment that interacts with the Cas12a polypeptide.
34. The method of any one of embodiments 24-33, wherein the target site is located immediately 3′ of a PAM site in the genome of the plant cell.
35. The method of embodiment 34, wherein the PAM site comprises TTTV.
36. The method of any one of embodiments 24-35, wherein the introducing is at a temperature from about 22° C. to about 32° C. or about 22° C. to about 25° C., optionally wherein the temperature is about 22° C. or about 25° C.
37. The method of any one of embodiments 24-36, wherein the polynucleotide sequence encoding the Cas12a polypeptide is codon-optimized for expression in a plant cell.
38. The method of any one of embodiments 24-37, wherein the polynucleotide encoding the Cas12a polypeptide is present in a vector.
39. The method of any one of embodiments 24-38, further comprising regenerating a plant from the plant cell.
40. A plant cell produced by the method of any one of embodiments 24-39.
41. A plant comprising the plant cell of embodiment 40.
42. A nucleic acid construct comprising a polynucleotide encoding a Cas12a polypeptide, wherein the Cas12a polypeptide has at least 80%, at least 90%, at least 95%, at least 98%, or at least 99% sequence identity to the amino acid sequence set forth in any of SEQ ID NOs: 18-34, wherein the polynucleotide is operably linked to a heterologous promoter that is operable in a plant cell.
43. The nucleic acid construct of embodiment 42, wherein the polynucleotide encoding the Cas12a polypeptide has at least 80%, at least 90%, at least 95%, at least 98%, or at least 99% sequence identity to the nucleotide sequence set forth in any of SEQ ID NOs: 1-17.
44. The nucleic acid construct of embodiment 42 or embodiment 43, wherein the Cas12a polypeptide comprises the amino acid sequence set forth in any of SEQ ID NOs: 18-34.
45. The nucleic acid construct of any one of embodiments 42-44, wherein the polynucleotide encoding the Cas12a polypeptide comprises the nucleotide sequence set forth in any of SEQ ID NOs: 1-17.
46. The nucleic acid construct of any one of embodiments 42-45, wherein the heterologous promoter is an inducible promoter, a constitutive promoter, a cell type-specific promoter, or a developmentally preferred promoter.
47. The nucleic acid construct of any one of embodiments 42-46, wherein the polynucleotide encoding the Cas12a polypeptide is codon-optimized for expression in a plant cell.
48. The nucleic acid construct of any one of embodiments 42-47, wherein the Cas12a polypeptide has reduced or eliminated nuclease activity.
49. The nucleic acid construct of any one of embodiments 42-48, wherein the Cas12a polypeptide has nickase activity.
50. The nucleic acid construct of any one of embodiments 42-49, wherein the polynucleotide encodes a Cas12a polypeptide fused to a transcriptional activation domain, a transcriptional repression domain, a deaminase domain, or a reverse transcriptase domain.
51. A plant or a plant cell comprising the nucleic acid construct of any one of embodiments 42-50.
52. A plant or a plant cell comprising: (i) a Cas12a polypeptide, or a polynucleotide encoding the Cas12a polypeptide, wherein the Cas12a polypeptide has at least 80%, at least 90%, at least 95%, at least 98%, or at least 99% sequence identity to the amino acid sequence set forth in any of SEQ ID NOs: 18-34; and (ii) a DNA-targeting RNA, or a DNA polynucleotide encoding a DNA-targeting RNA, wherein the DNA-targeting RNA is capable of forming a complex with the Cas12a polypeptide and directing the complex to a target site.
53. The plant or plant cell of embodiment 52, wherein the polynucleotide encoding the Cas12a polypeptide has at least 80%, at least 90%, at least 95%, at least 98%, or at least 99% sequence identity to the nucleotide sequence set forth in any of SEQ ID NOs: 1-17.
54. The plant or plant cell of embodiment 52 or embodiment 53, wherein the Cas12a polypeptide comprises the amino acid sequence set forth in any of SEQ ID NOs: 18-34.
55. The plant or plant cell of any one of embodiments 52-54, wherein the polynucleotide encoding the Cas12a polypeptide comprises the nucleotide sequence set forth in any of SEQ ID NOs: 1-17.
56. The plant or plant cell of any one of embodiments 52-55, wherein the plant is a monocotyledonous or a dicotyledonous plant.
57. The plant or plant cell of any one of embodiments 52-56, wherein the plant is Oryza sativa. Solanum lycopersicum, or Populus sp.
58. The plant or plant cell of any one of embodiments 52-57, wherein expression of the Cas12a polypeptide is under the control of an inducible promoter, a constitutive promoter, a cell type-specific promoter, or a developmentally preferred promoter.
59. The plant or plant cell of any one of embodiments 52-58, wherein the polynucleotide sequence encoding the Cas12a polypeptide is codon-optimized for expression in a plant cell.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Identification and Selection of Novel Cas12a Orthologs

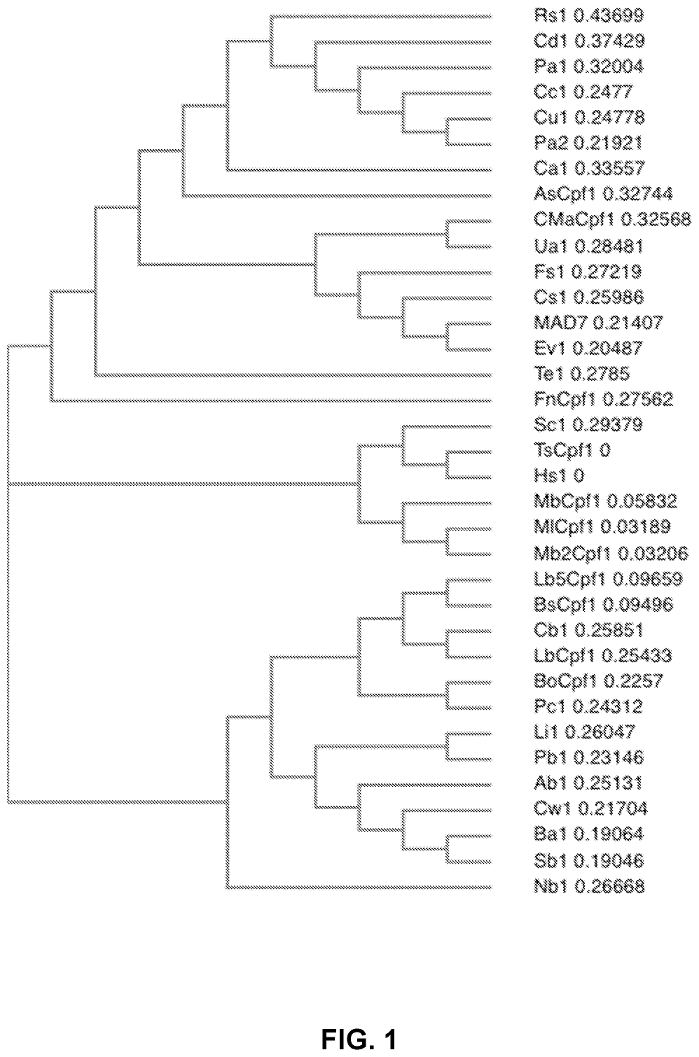

Cas12a ortholog candidates were identified using LbCas12a, FnCas12a, ErCas12a, Mb2Cas12a as queries to BLAST against the Joint Genome Institute Microbial Genomes and Microbiomes database. Since genome editing in plants is mostly conducted at ambient temperatures, identifying Cas12a nucleases that are more tolerant to lower temperatures was of interest. Hence, Cas12a nucleases originating from bacteria with ambient living temperatures and capability to reproduce in soil were selected. The Cas12a nucleases all have estimated length of more than 1000 amino acids and less than 80% sequence homology to queries. Seventeen Cas12a orthologs were identified and selected to test in plants: Ba1Cas12a, Ca1Cas12a, Cb1Cas12a, Cc1Cas12a, Cu1Cas12a, Ev1Cas12a, Hs1Cas12a, Pa2Cas12a, Pc1Cas12a, Sc1Cas12a, Bb1Cas12a, Cd1Cas12a, Cw1Cas12a, Mb1Cas12a, NbCas12a, PbCas12a, and UaCas12a (FIG. 1).

### Example 2: Screening of Cas12a Orthologs in Rice Protoplasts

To test the editing efficiency of the Cas12a orthologs, rice protoplast transformations targeting six sites with TTTV and TTV PAMs were conducted. All seventeen Cas12a genes were synthesized with rice codon optimization and cloned into pYPQ230 at the NotI and NcoI sites to construct the Cas12a entry clones. Four crRNAs were used to target six sites in the rice genome: L9 targeting OsEPFL9 with the TTTV PAM; CC1 targeting Os02g46610 with the TTV PAM and Os04g50120 with the TTTV PAM: GG1 targeting Os12g24050 with the TTV PAM and Os01g23900 with the TTTV PAM: CG2 targeting Os07g40790 with the TTV PAM and Os07g40404 with the TTTV PAM. The crRNAs were clones into pYPQ141-ZmUbi-RZ-Fn at the BsmBI site to construct the crRNA entry clones. Cas12a entry clones and crRNA entry clones were assembled with the destination vector pYPQ203 by LR reactions to form the final T-DNA vectors.

Rice protoplasts were isolated and transformed as previously described (Tang et al. (2016) Mol Plant. 9 (7): 1088-91). Briefly, rice seedlings (Japonica cultivar Kitaake) were grown in dark for 14 days and leaves were cut into 0.5-1.0 mm strips and placed in the enzyme solution. After incubation at 28° C. for 8 hours in dark, rice protoplasts were filtered through the 75 μm cell strainer and washed with W5 buffer twice. Protoplasts were resuspended in the MMG buffer, and the concentration were adjusted to 2×10⁶ cells/mL. 200 μL protoplasts were mixed with 30 μg (30 μL) plasmid, and then mixed with 230 μL polyethylene glycol (PEG). The mixture was set at room temperature for 30 minutes and then 900 μL W5 were added to stop the transformation. Protoplasts were collected and resuspended in 1.5 mL W5 buffer and incubated at 32° C. or 25° C. in dark for 2 days. Protoplasts transformed with water were used as the wild type (WT) control for all genome editing experiments.

Protoplast were lysed and target sites were amplified using the Phire Plant Direct PCR Kit (Thermo Scientific). PCR products were digested with corresponding enzymes and run on 2% agarose gel for Restriction Fragment Length Polymorphism (RFLP) analysis. Editing efficiencies were calculated with ImageJ (imagej.nih.gov/ij/). For high-throughput next generation sequencing (NGS), target sites were amplified with barcoded primers. Pooled amplicons were subjected for sequencing using the HiSeq2500 platform. Editing efficiencies, deletion size and deletion position profiles were analyzed using CRISPRMatch.

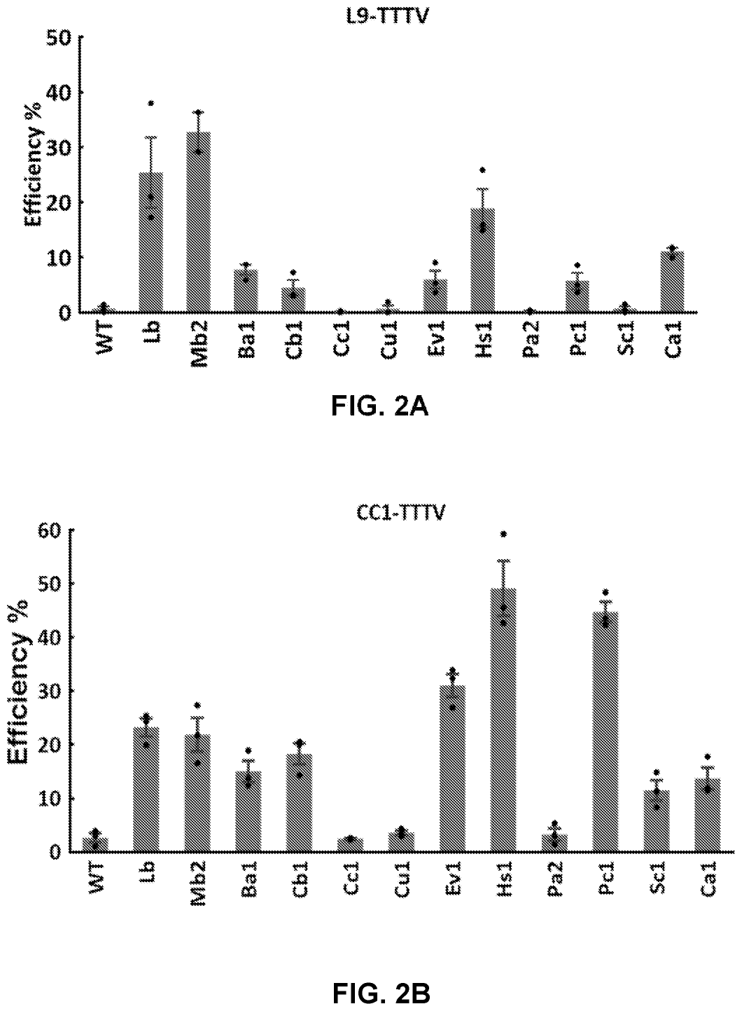

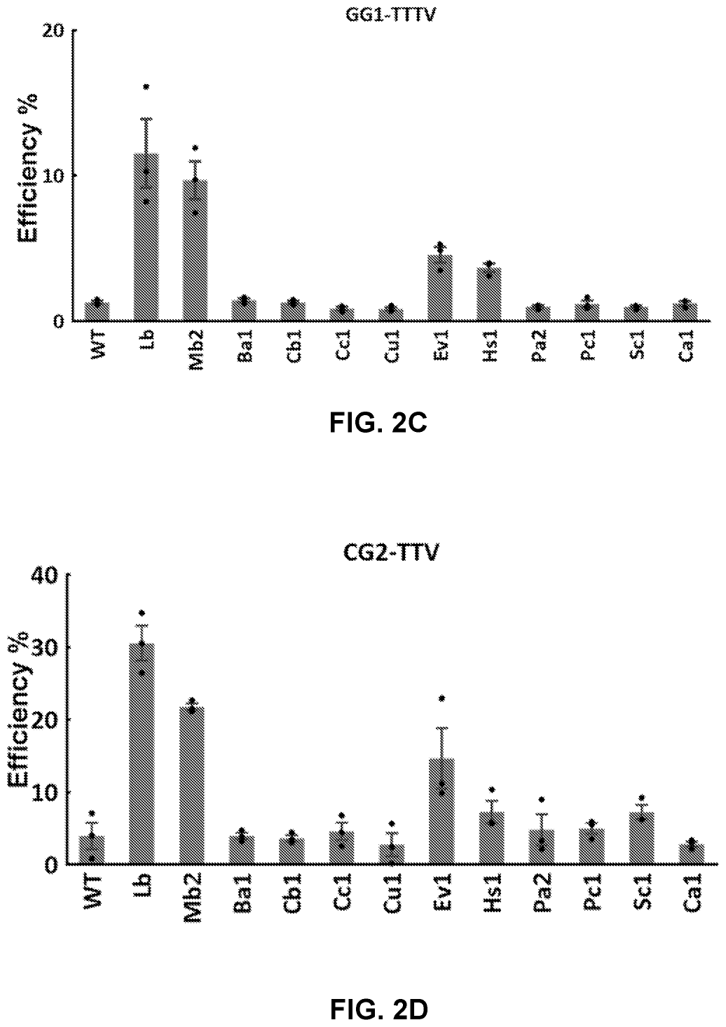

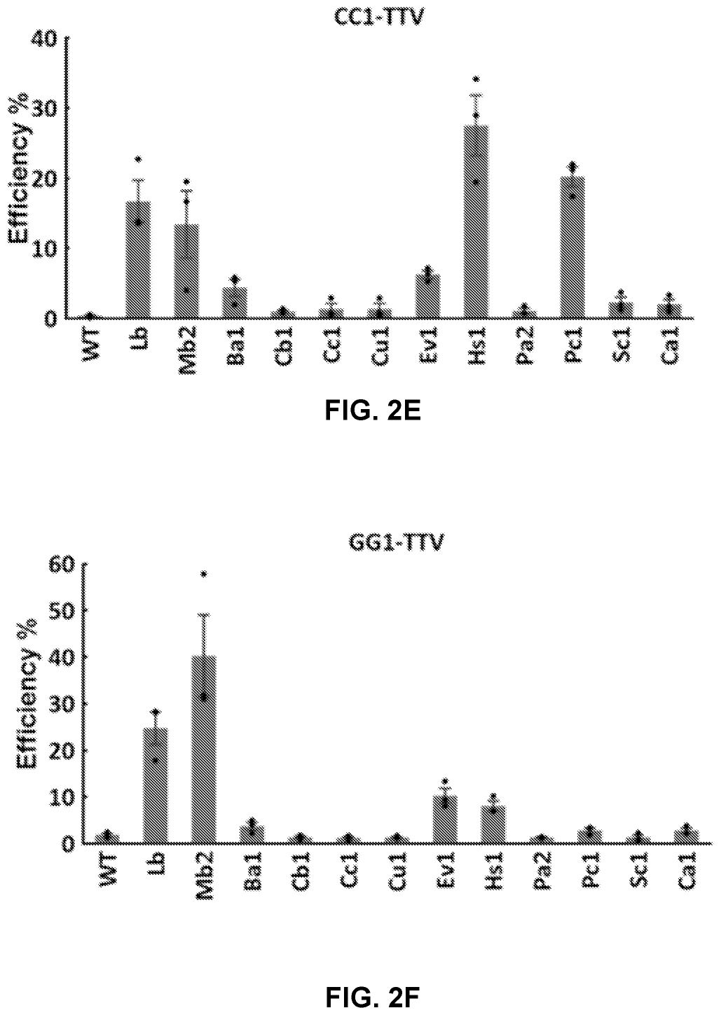

Screening of ten Cas12a orthologs showed they had variable genome editing activities across these six target sites. Seven of these Cas12a nucleases (BaCas12a, CbCas12a, Ev1Cas12a, Hs1Cas12a, PcCas12a, Sc1Cas12a and Ca1Cas12a) showed comparable or higher genome editing efficiencies than LbCas12a and Mb2Cas12a at the CC1-TTTV target site (FIG. 2A-F). At this CC1-TTTV site, the average editing efficiencies for Ev1Cas12a, HsCas12a, and PcCas12a reached 31.05%, 49.17% and 44.76%, respectively, which were higher than LbCas12a and Mb2Cas12a. These three Cas12a nucleases (Ev1Cas12a, HsCas12a, and PcCas12a) were able to reliably edit both TTTV and TTV PAM sites using the RFLP analysis.

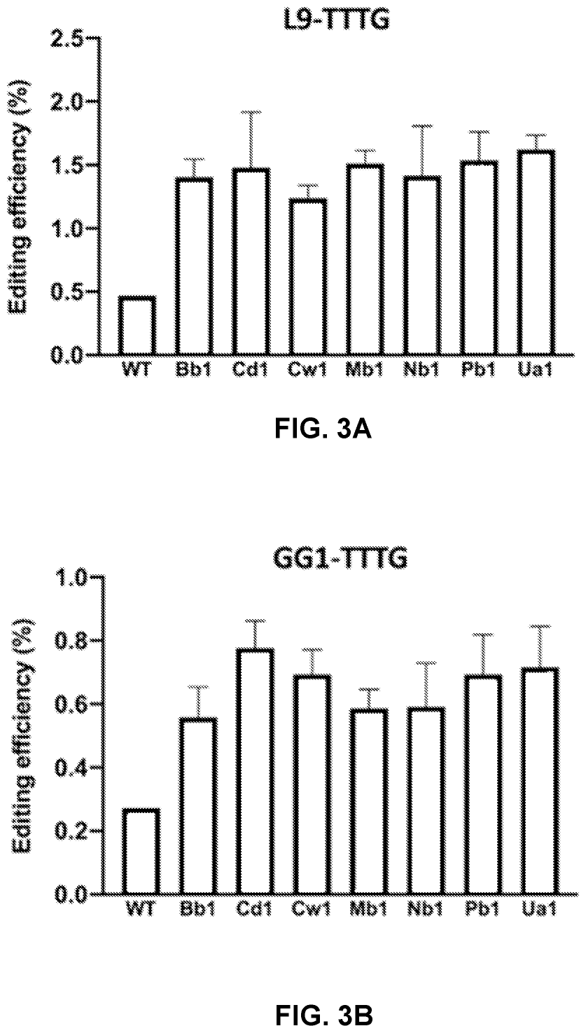

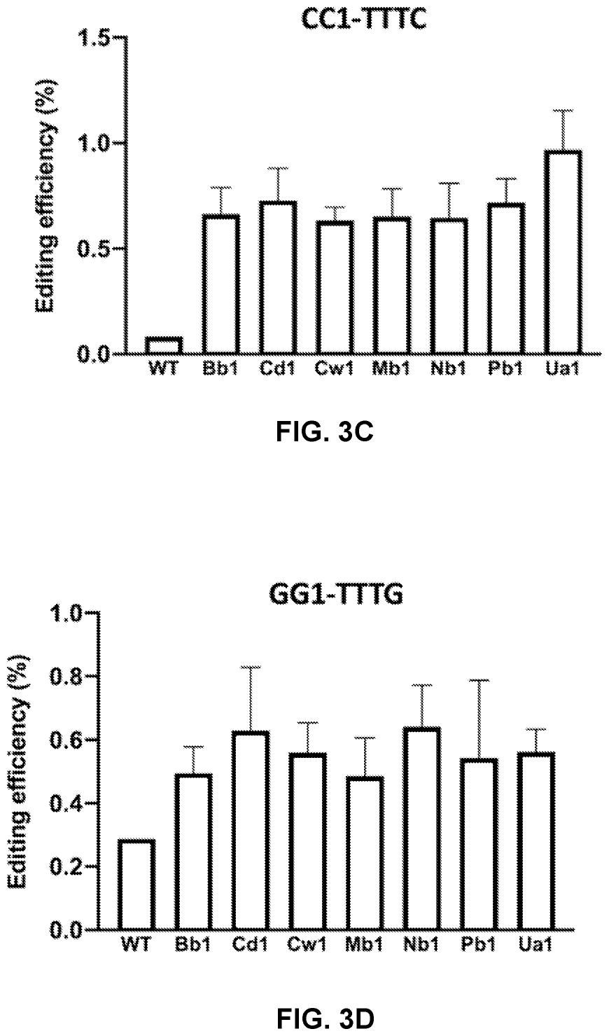

The remaining seven Cas12a orthologs were tested at four target sites in rice protoplast at 32° C. All seven Cas12a orthologs enabled genome editing at four target sites with editing efficiency of 0.5%-2% (FIG. 3A-D). Deletion size for these seven Cas12a orthologs is mainly 1 bp.

Low genome editing activities in certain Cas12a orthologs may be attributed to the crRNA of FnCas12a being used in these experiments. The predicted crRNAs for some of the Cas12a orthologs are distinct from the crRNA of FnCas12a (SEQ ID NOs: 39-53). Genome editing capability and efficiency may be further optimized when each of the Cas12a orthologs is coupled with their respective crRNAs.

Taken together, EvlCas12a, HsCas12a, and PcCas12a were selected for further studies.

### Example 3: Genome Editing in Rice and Tomato Protoplasts

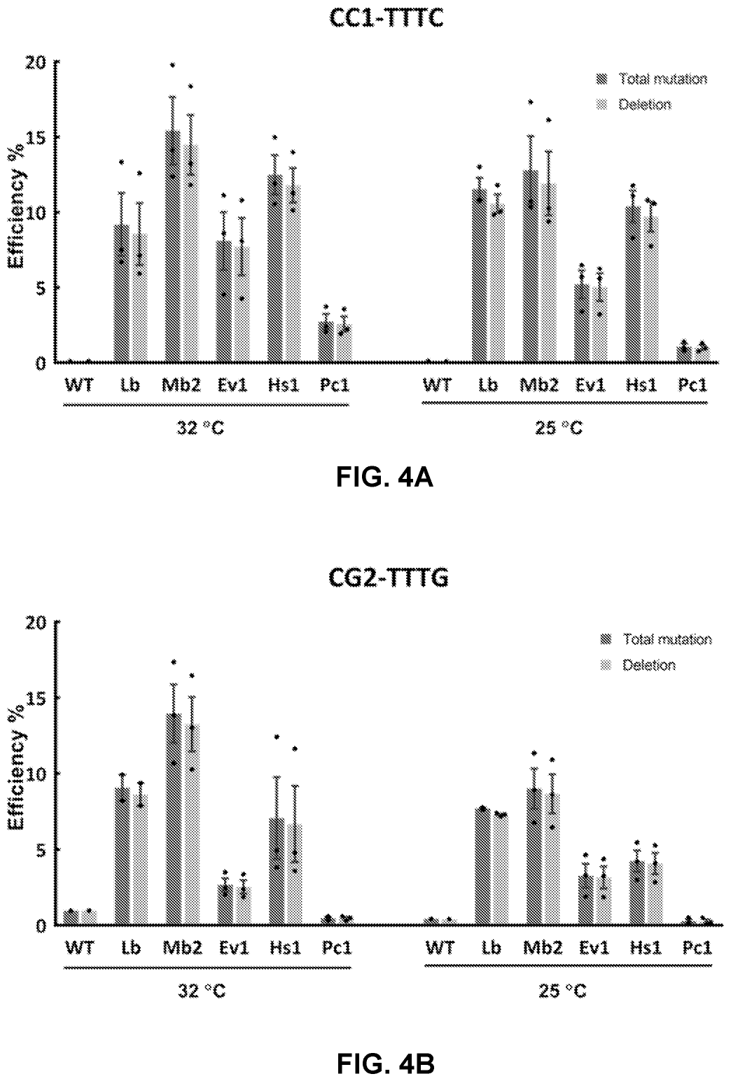

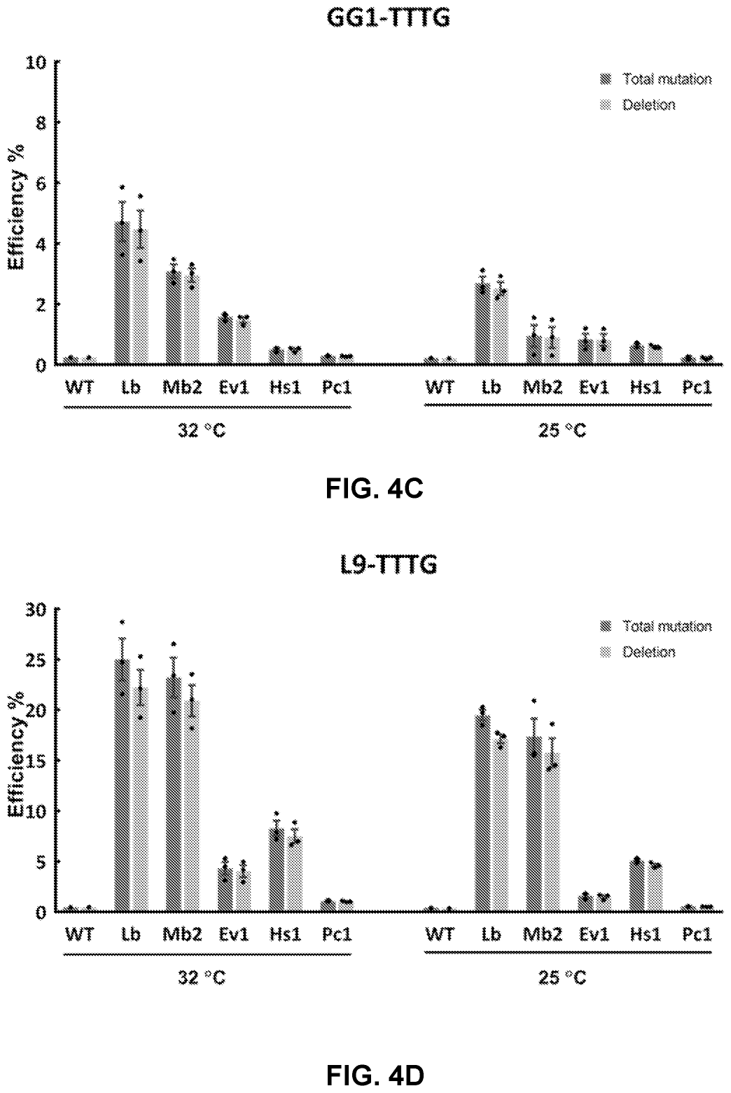

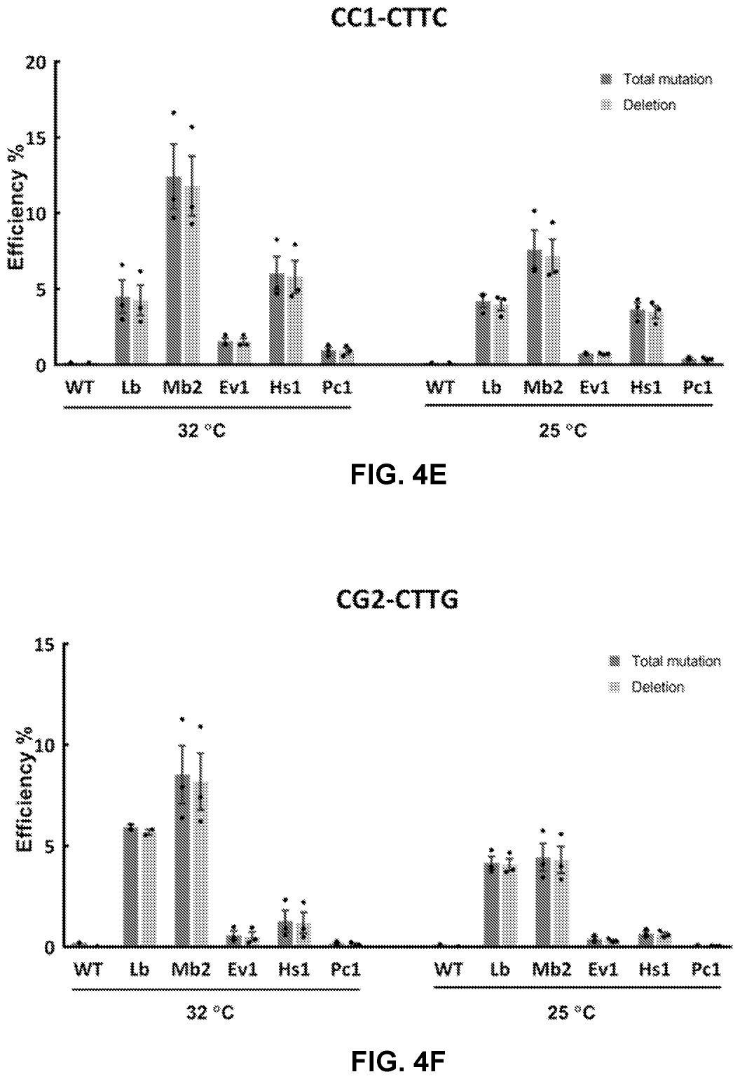

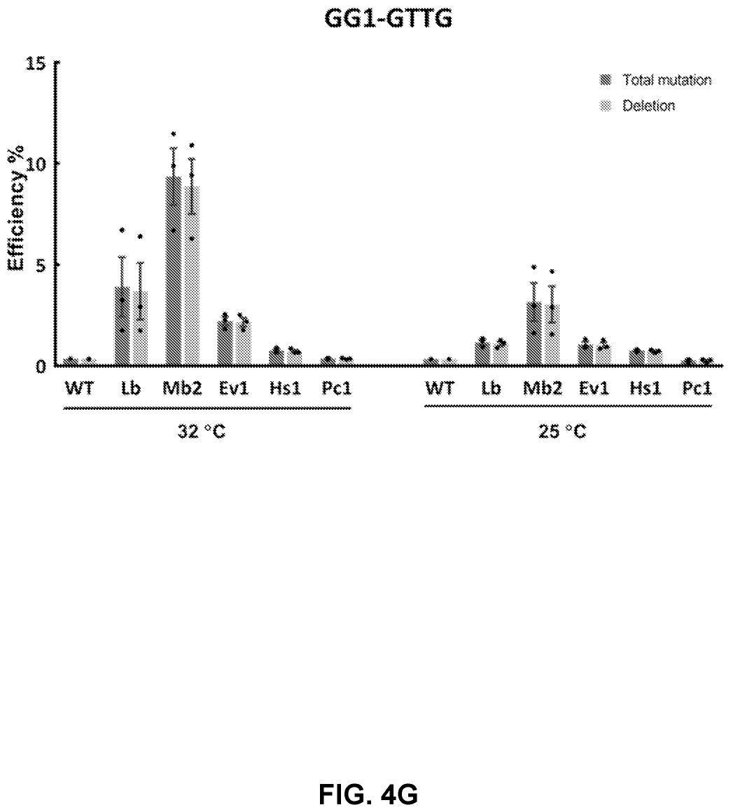

To further investigate the editing activity of Ev1Cas12a, HsCas12a, and PcCas12a, another round of rice protoplast transformations targeting seven sites at both 32° C. and 25° C. were conducted. PCR amplicon sequencing revealed that all three Cas12a orthologs showed slightly higher editing efficiency at 32° C. than at 25° C. (FIG. 4A-F), indicating they are not very sensitive to low temperatures. Ev1Cas12a and HsCas12a were able to edit most of the target sites, while Pc1Cas12a showed lower activities for most of the target sites. In general, these three Cas12a orthologs have a preference to edit TTTV PAM sites.

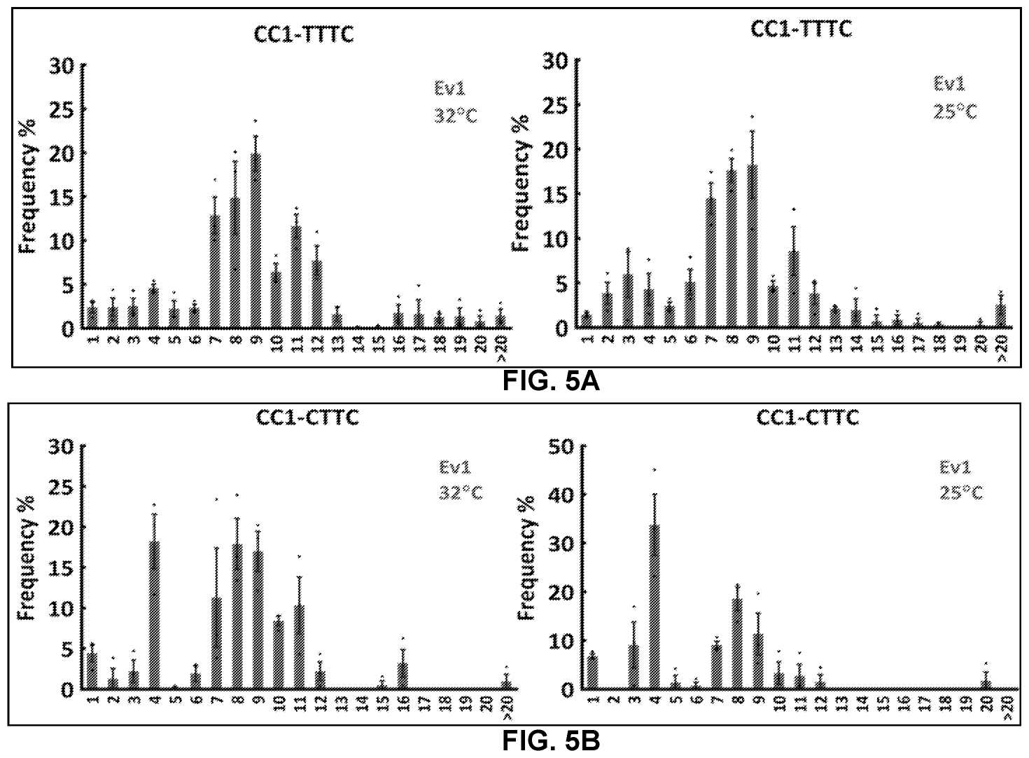

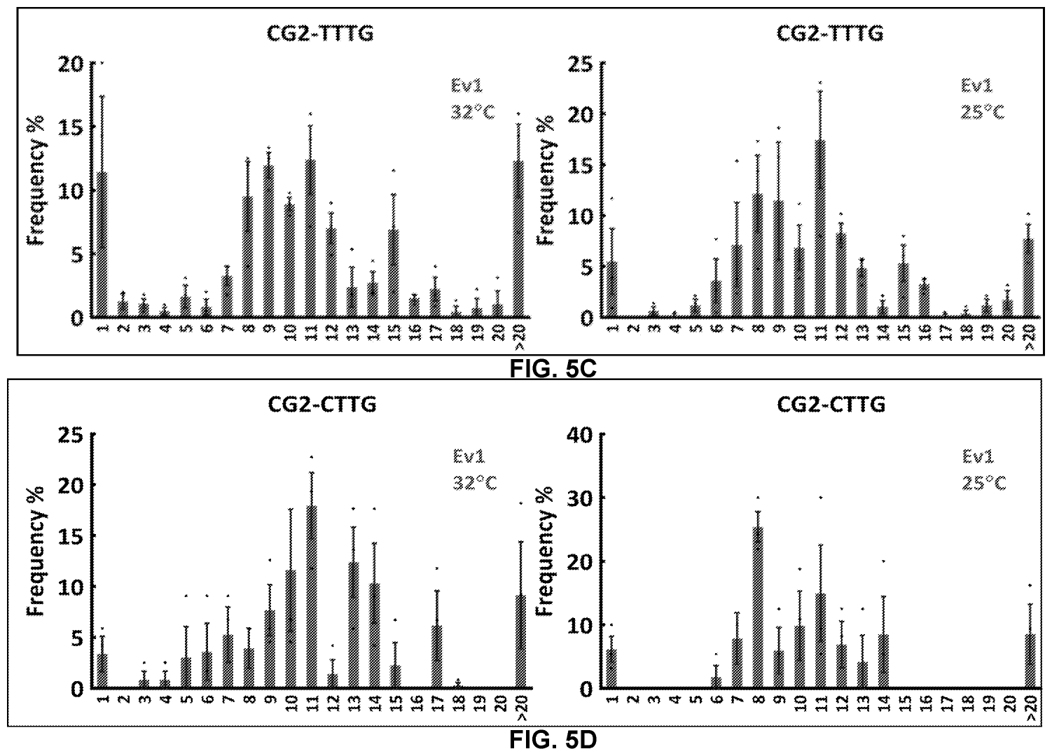

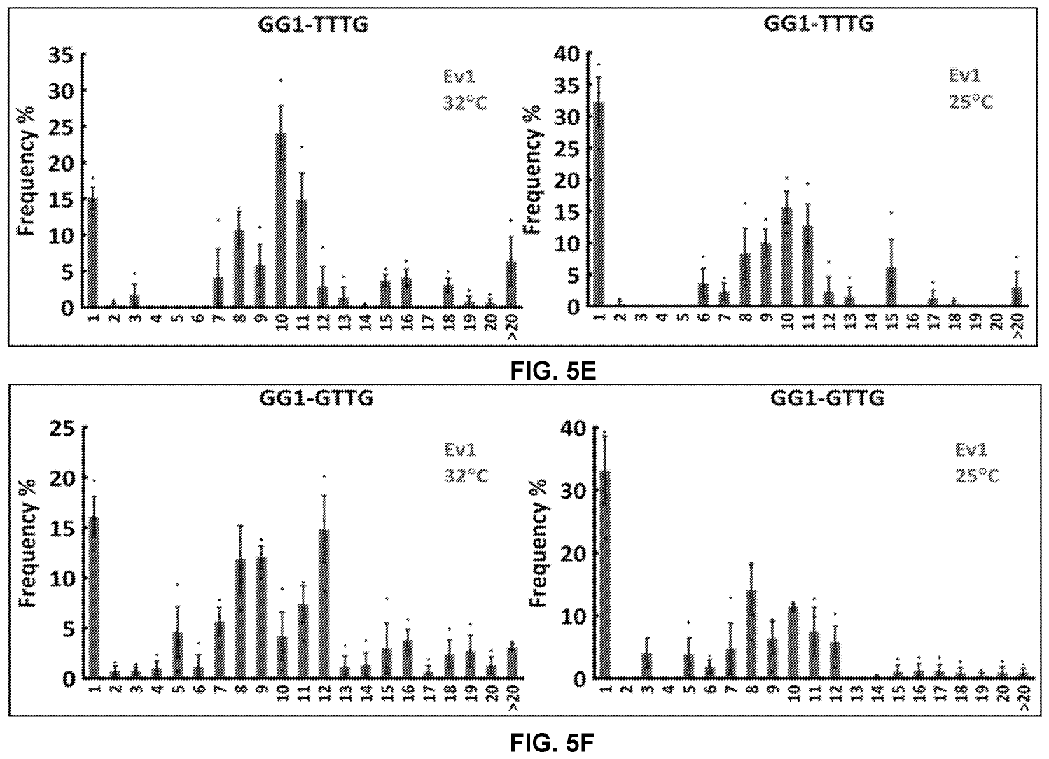

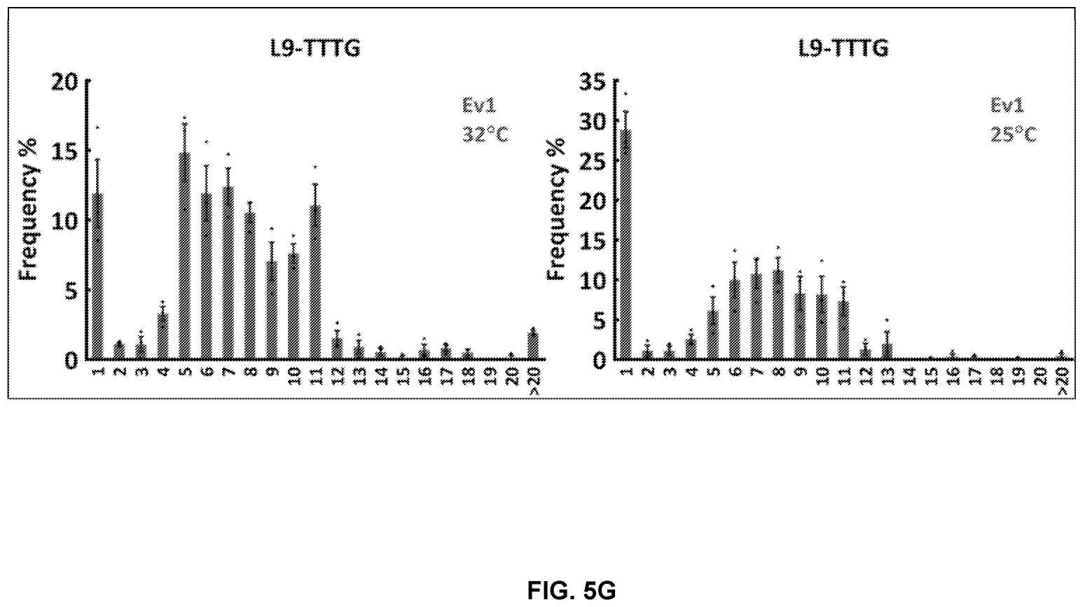

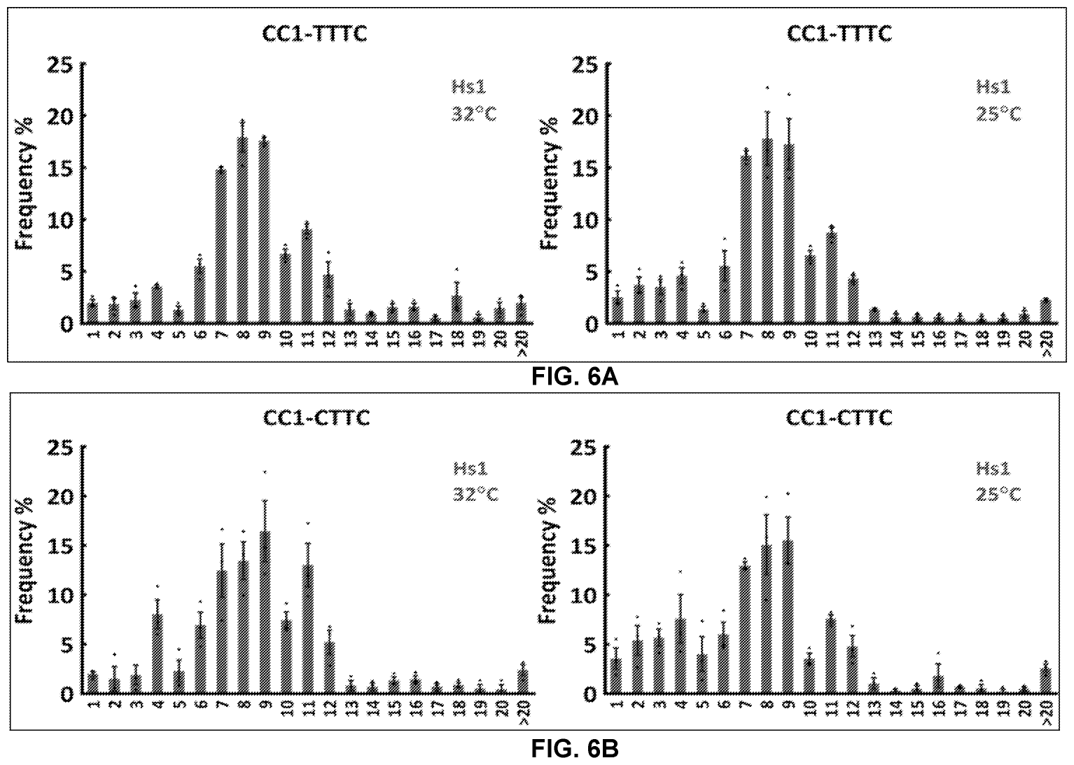

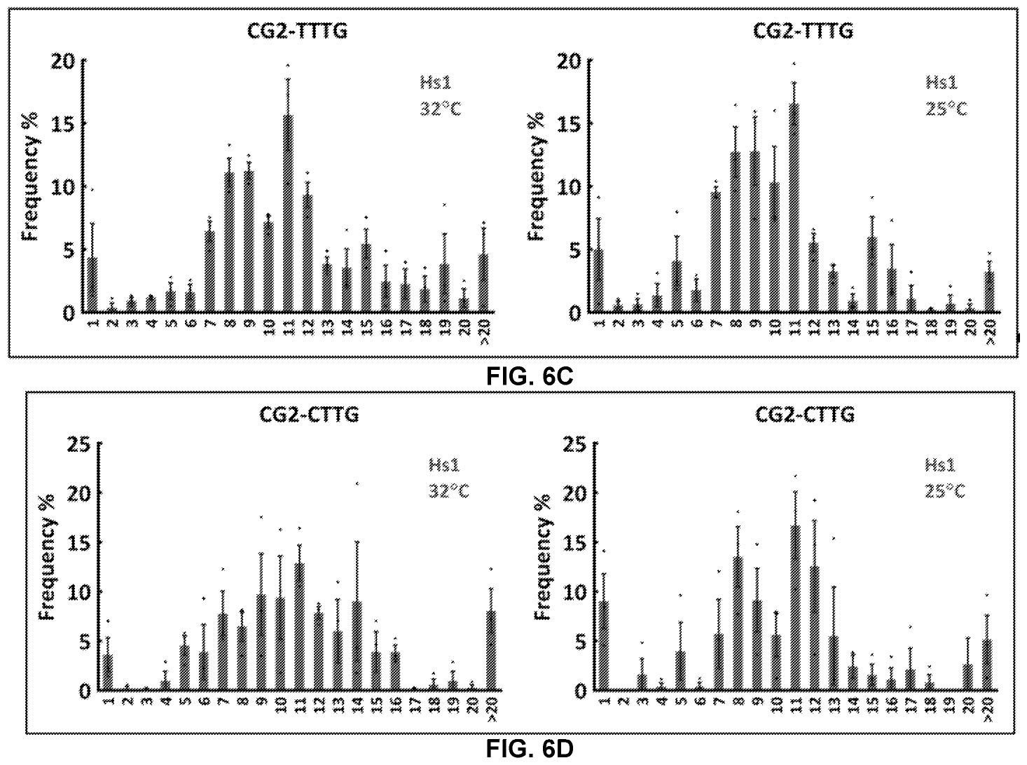

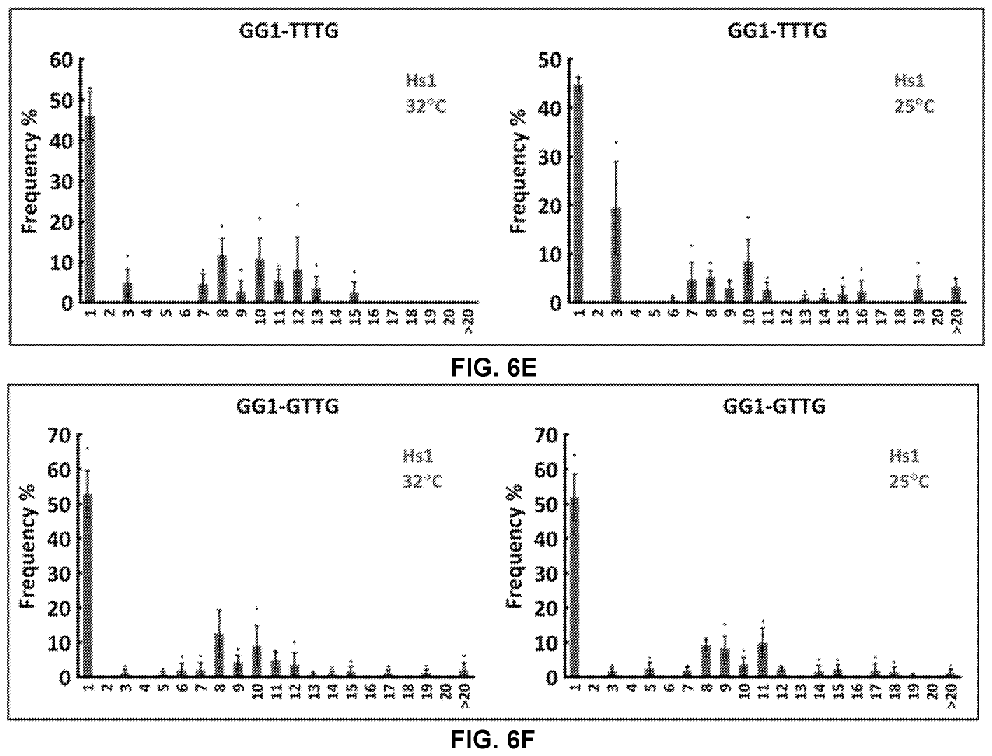

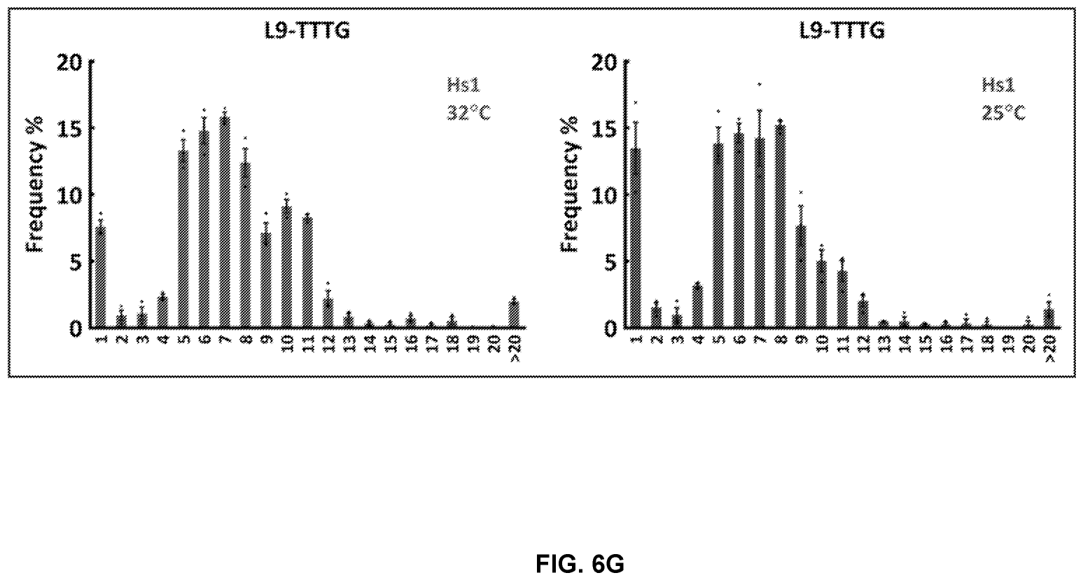

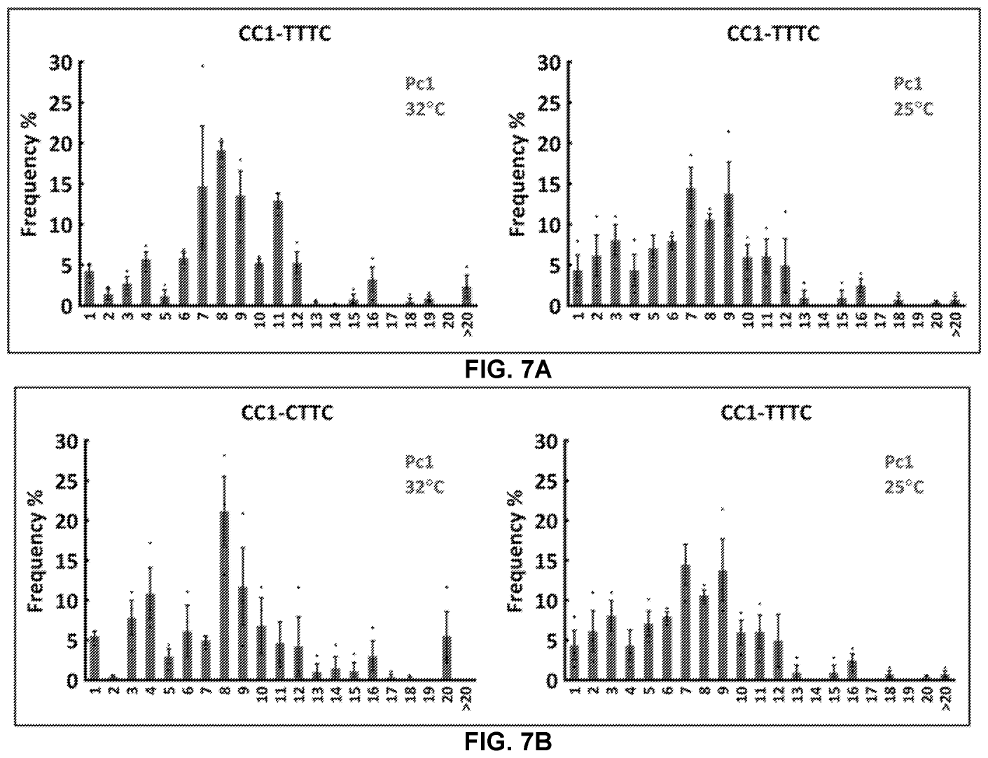

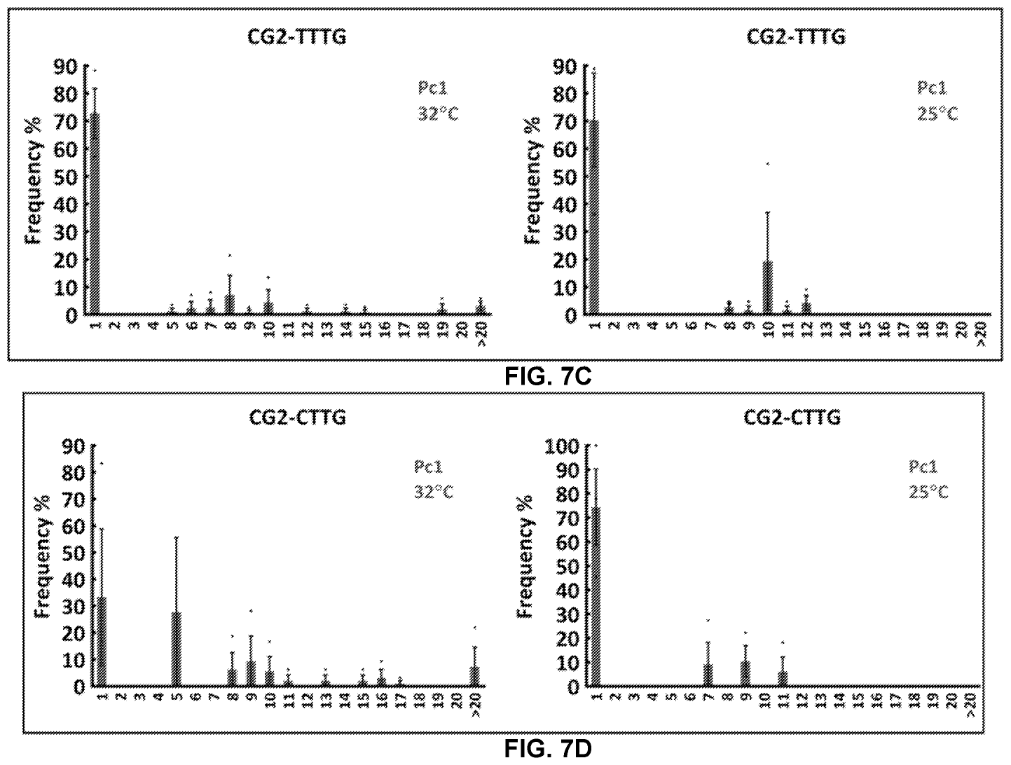

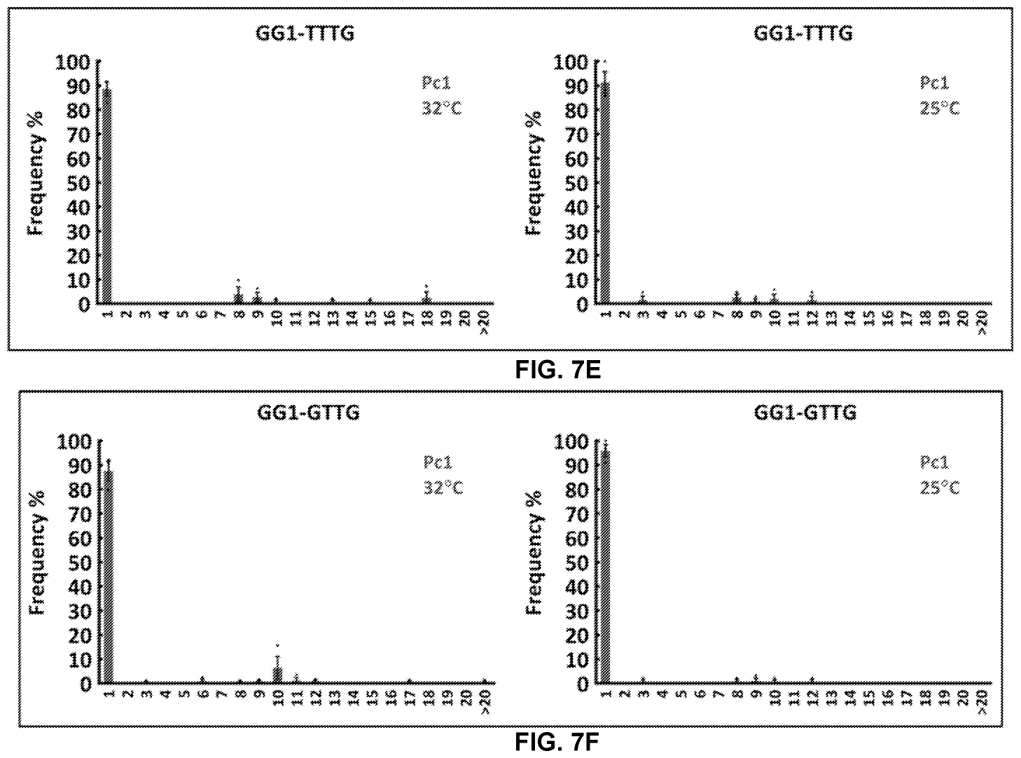

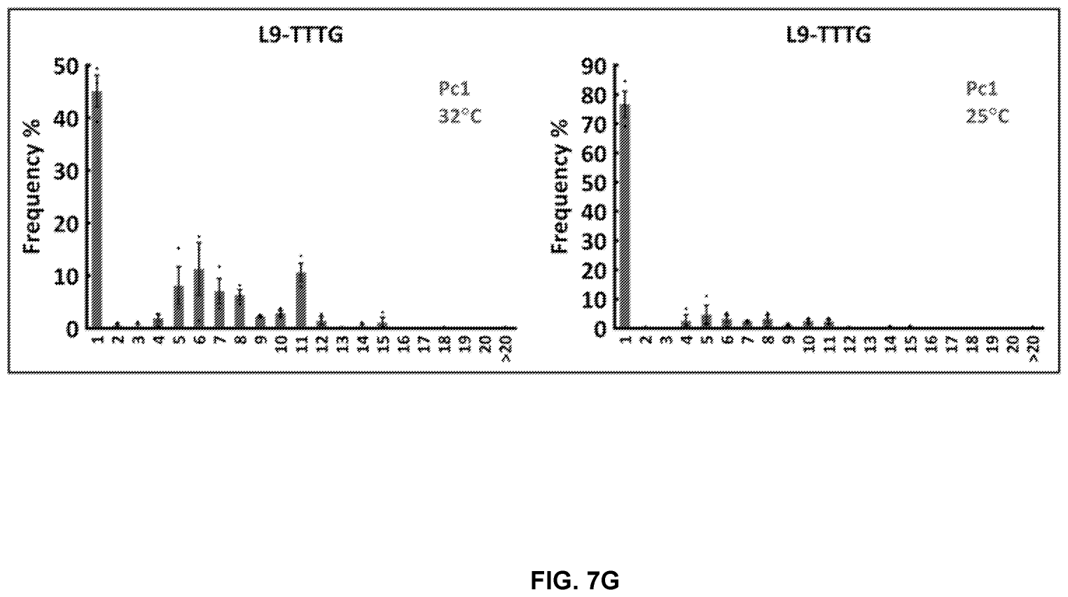

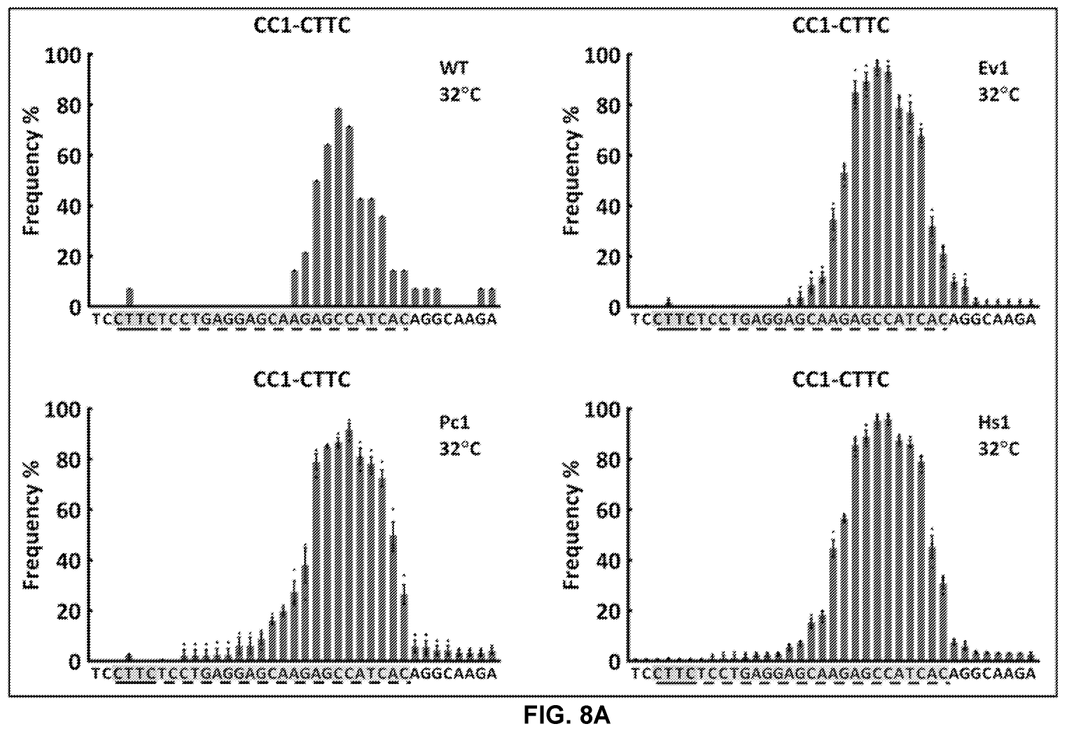

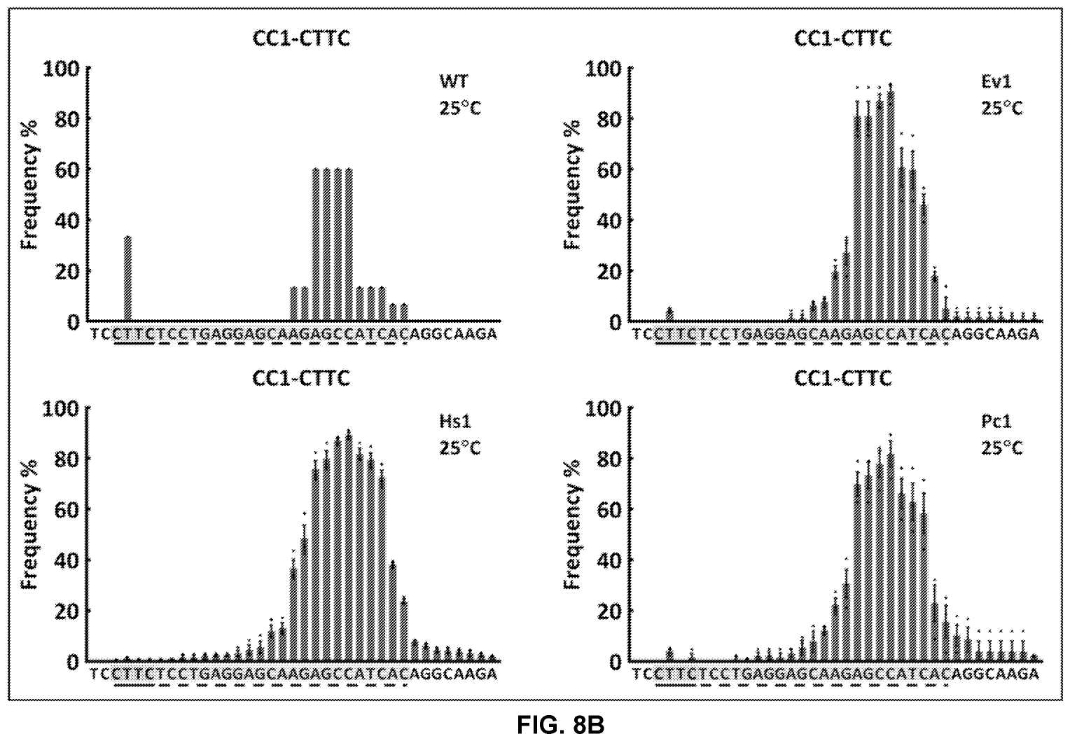

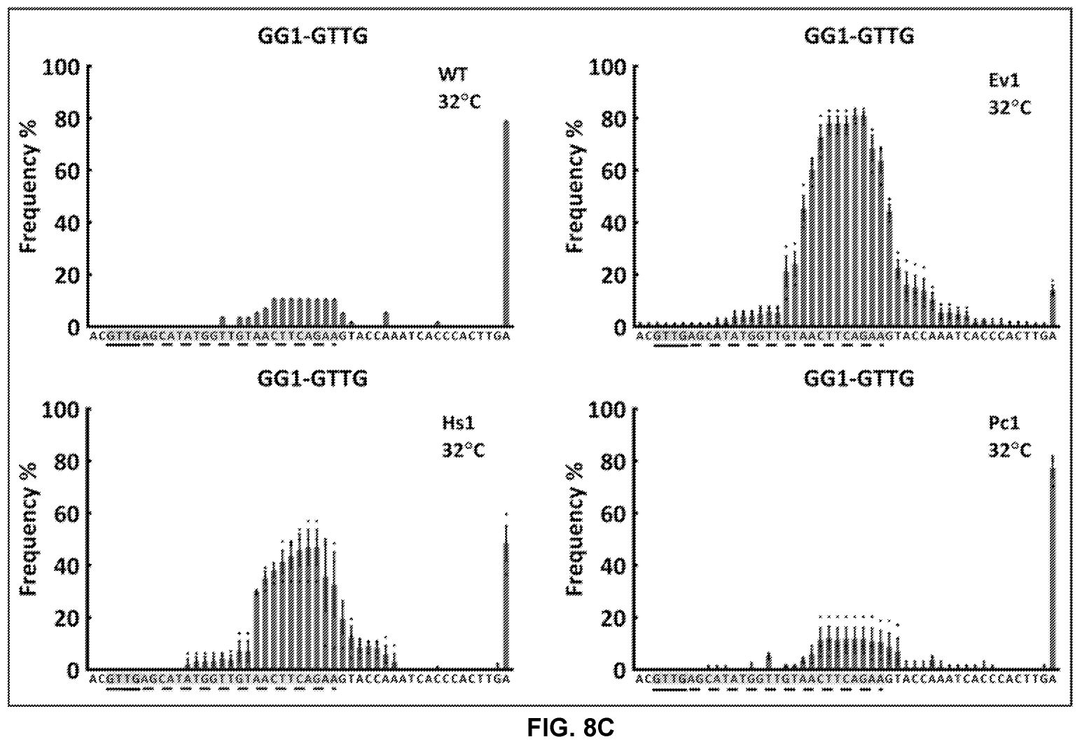

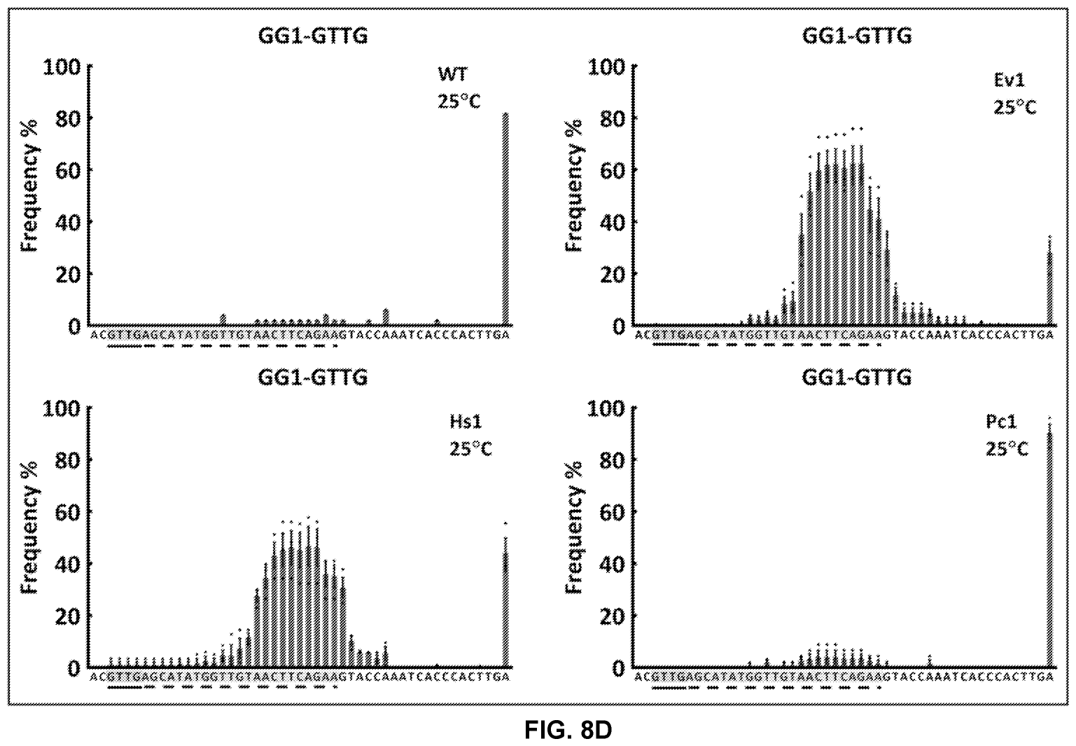

The analysis of NGS data revealed the editing profiles of the three Cas12a orthologs. As expected, the majority of edits were deletions (FIG. 5A-G. FIG. 6A-G, and FIG. 7A-G). Deletion sizes ranged from 4-12 bp for all three Cas12a orthologs (FIG. 8A-B), which is consistent with other Cas12a nucleases previously described. Deletion size was not significantly affected by temperature. The three Cas12a orthologs also showed similar deletion position profiles, which are not affected by temperature. These data suggest that these Cas12a nucleases cleave targets in a similar fashion, creating staggered DNA DSBs at the PAM-distal sites.

To test whether Ev1Cas12a, HsCas12a, and PcCas12a can generate targeted mutations in dicot crops, experiments were conducted in tomatoes. Multiplexed genome editing was conducted by targeting six sites with TTTV PAMs in tomatoes. The same Cas12a entry clones for rice genome editing were used. A multiplexed crRNA entry clone was generated containing six crRNAs targeting five genes involved in lycopene synthesis and catabolismo including SGR1 (two crRNAs), lycopene ε-cyclase (LCY-E), beta-lycopene cyclase (Ble), lycopene β-cyclase1 (LCY-B1), and LCY-B2. A tandem ribozyme multiplexing system was used. The 2×35S promoter was used for crRNA expression. Cas12a entry clones and the multiplexed crRNA entry clone were assembled with the destination vector pMDC32 by LR reactions to form the final T-DNA vectors. These constructs were assessed in tomato protoplasts.

Tomato seedlings (M82) were grown with 16/8 light/dark photoperiod for 7-9 days until the cotyledons were fully expanded. Cotyledons were cut off and placed in the same enzyme solution used for rice. After incubation at 28° C. for 8 hours in dark, tomato protoplasts were filtered through the 75 μm cell strainer and collected by centrifugation. 6 mL 0.55 M sucrose solution was added to the protoplasts followed by 2 mL W5 on top. After centrifuging for 30 min at 200 g. protoplasts were collected at the interface. Protoplasts were moved to new tubes and washed with W5 buffer twice. Protoplasts were then resuspended in the MMG buffer and used for transformation using the same method for rice.

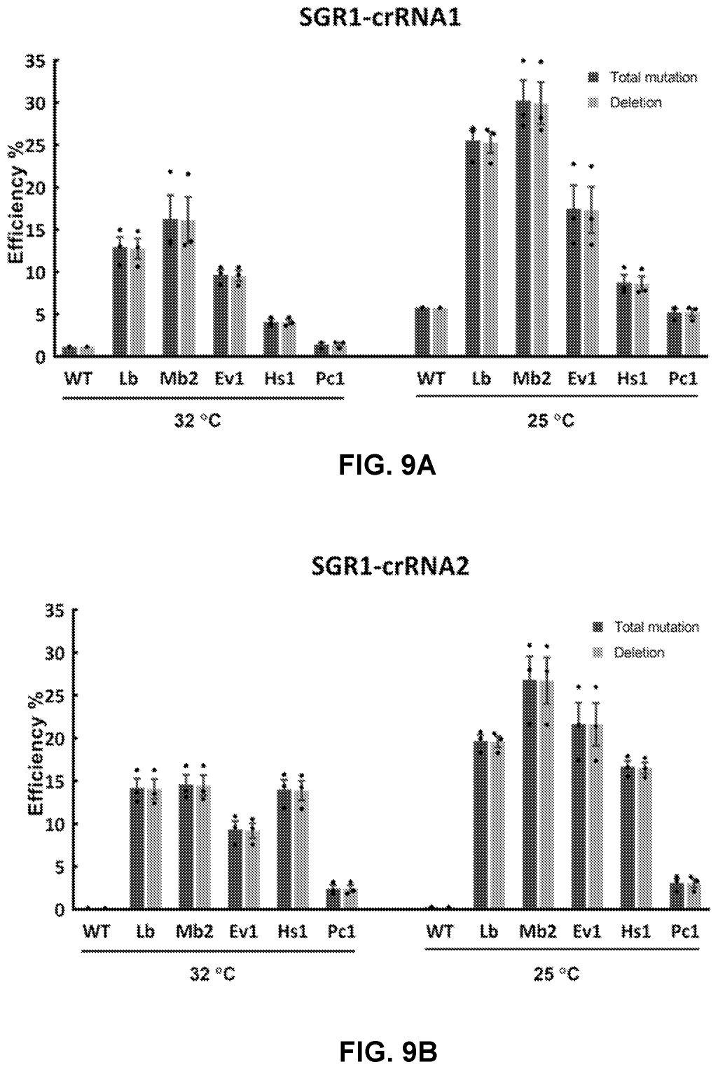

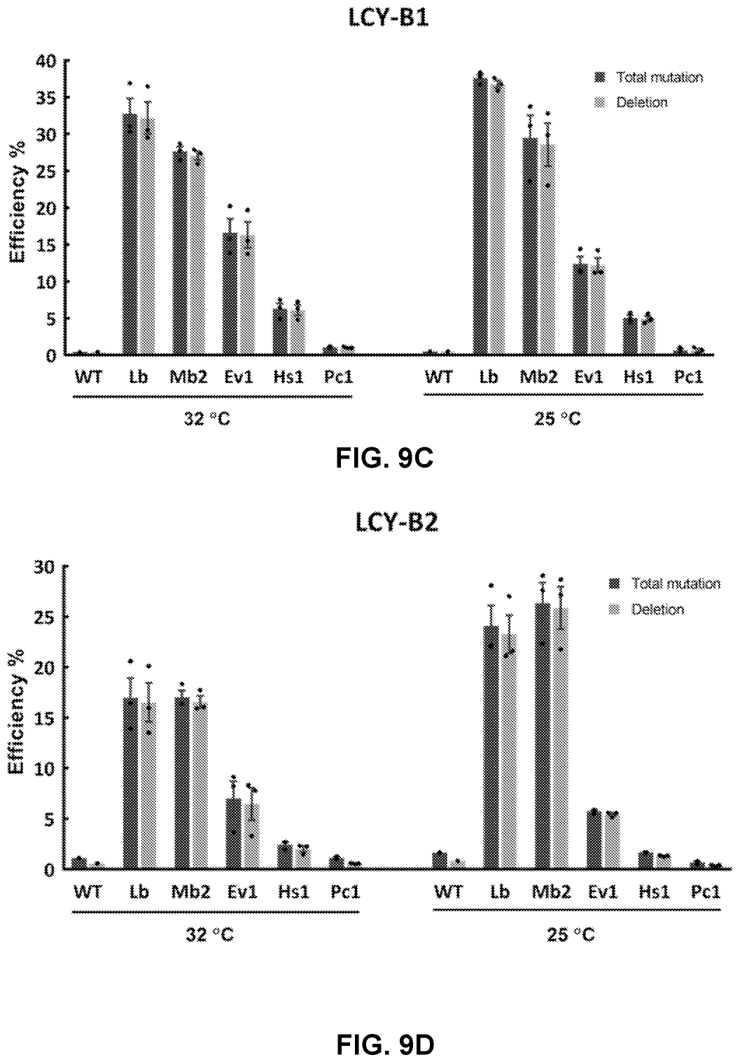

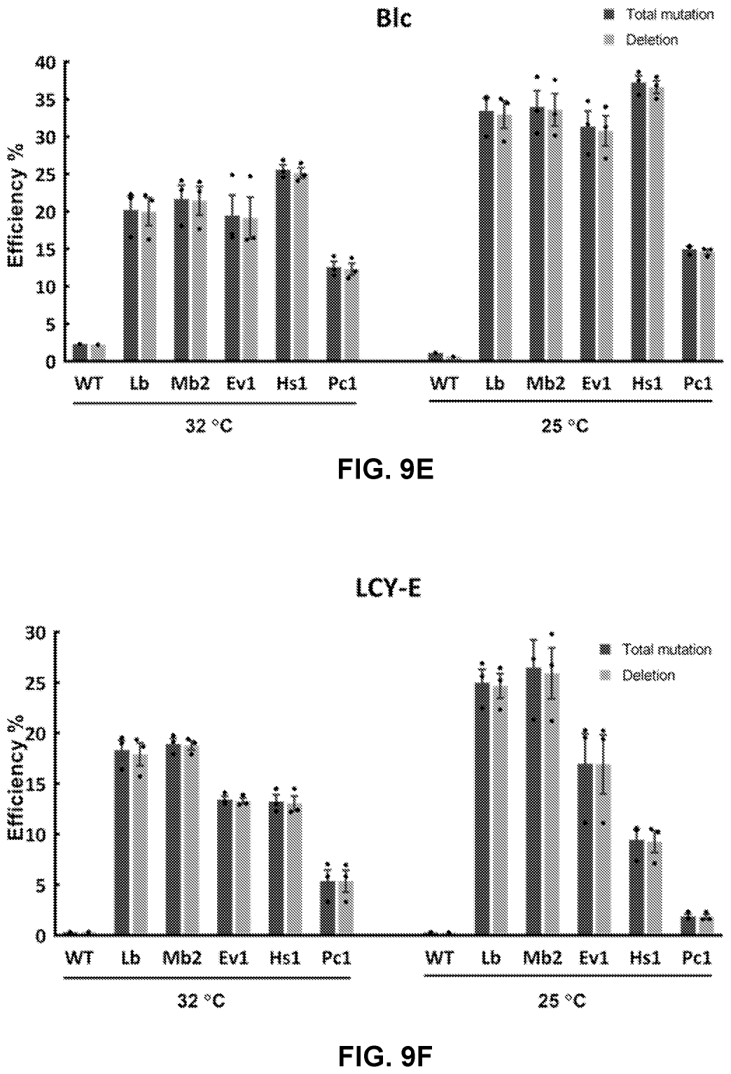

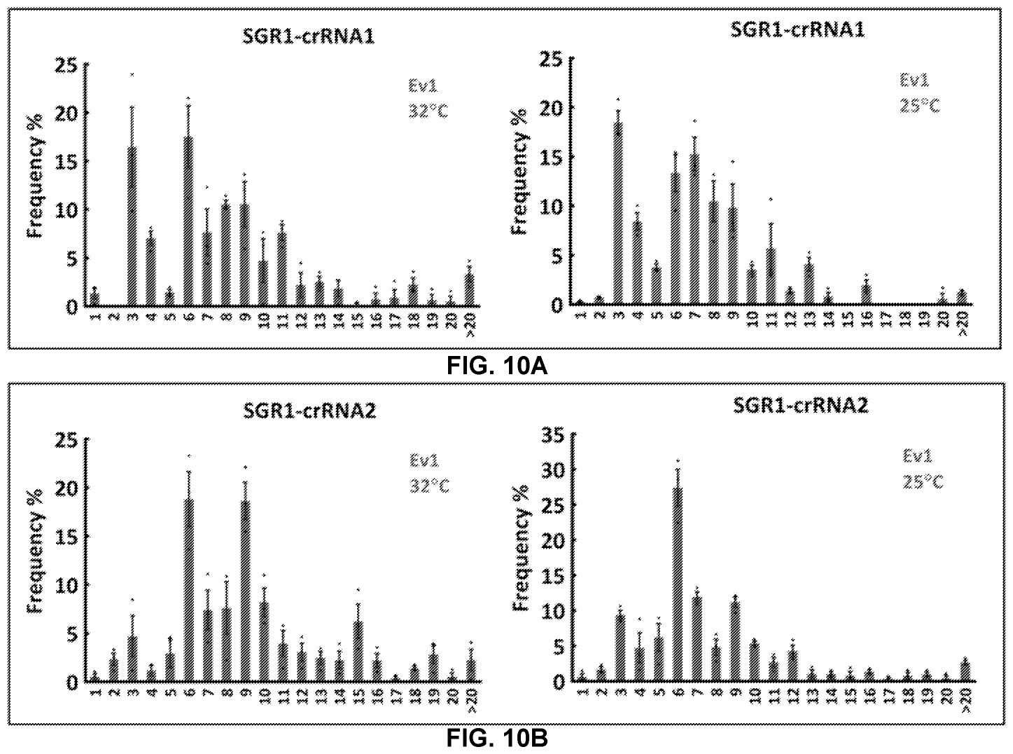

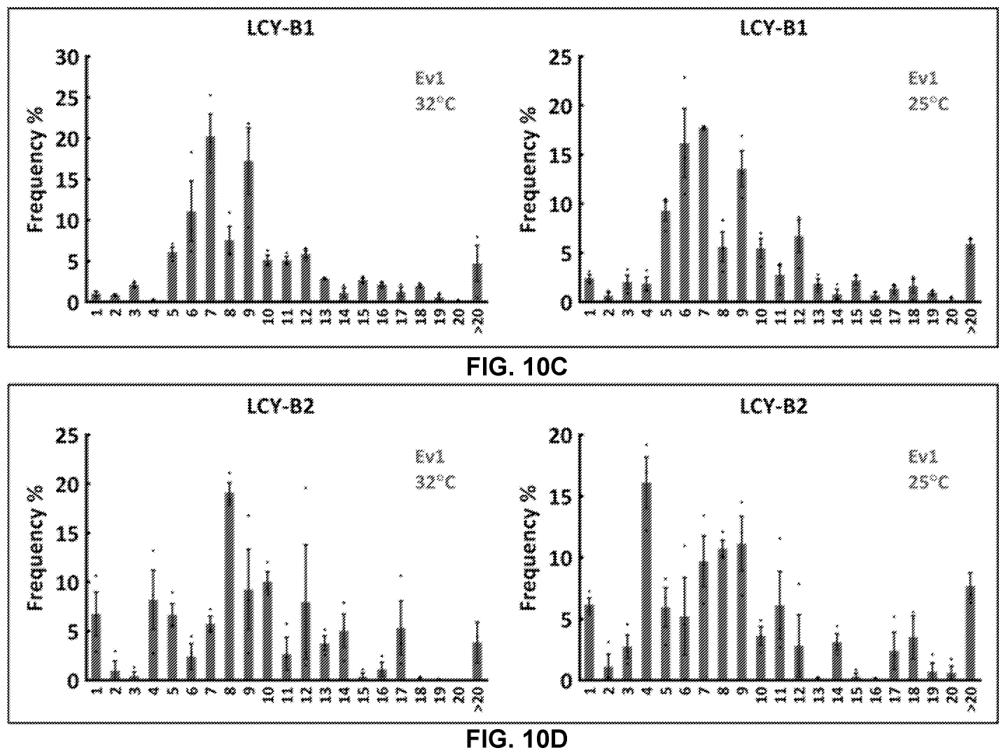

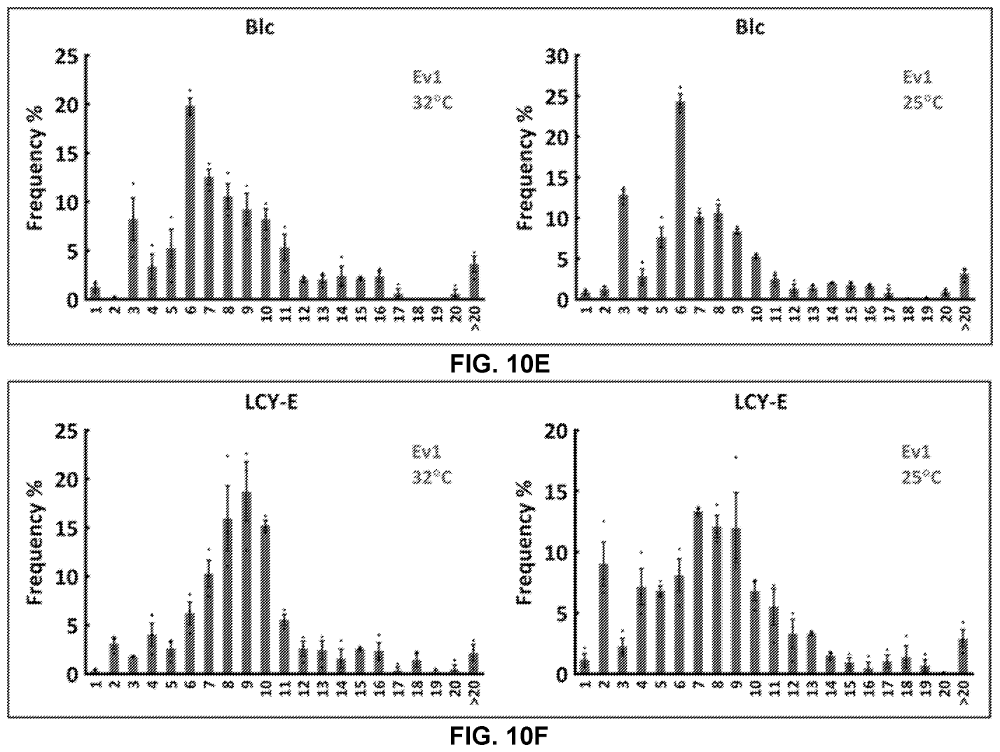

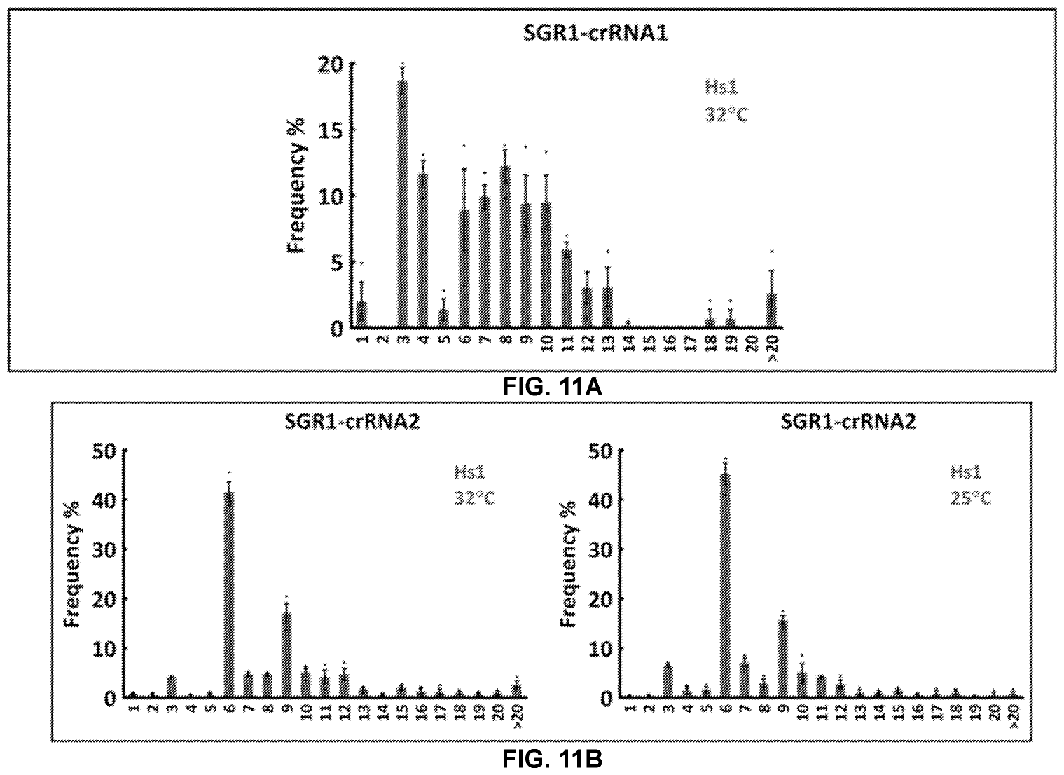

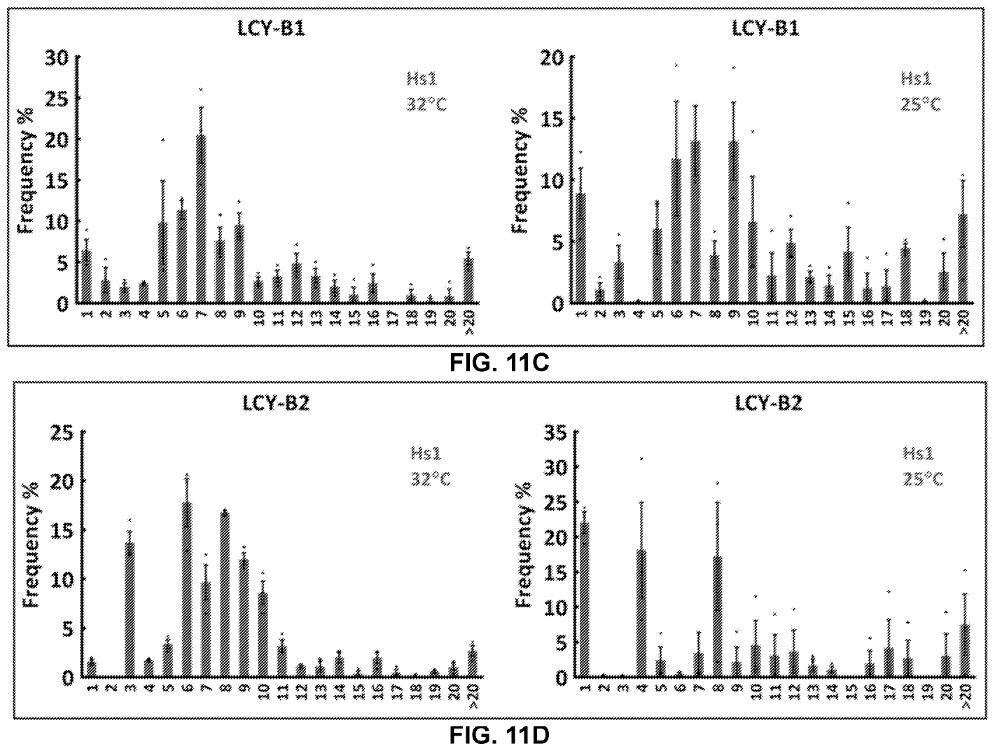

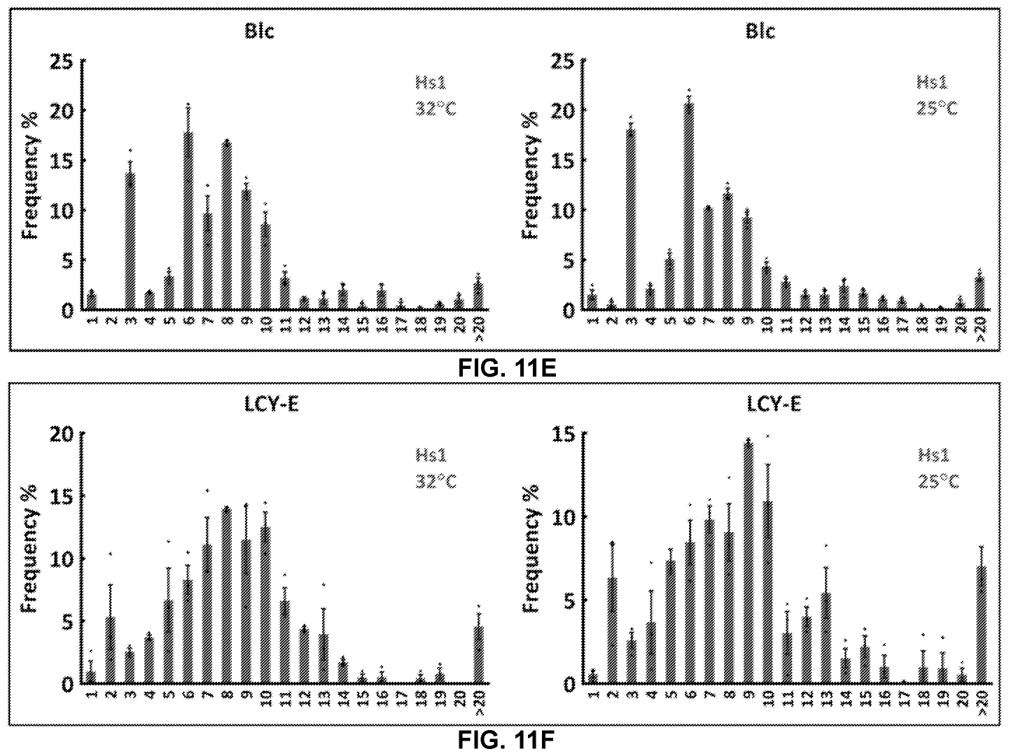

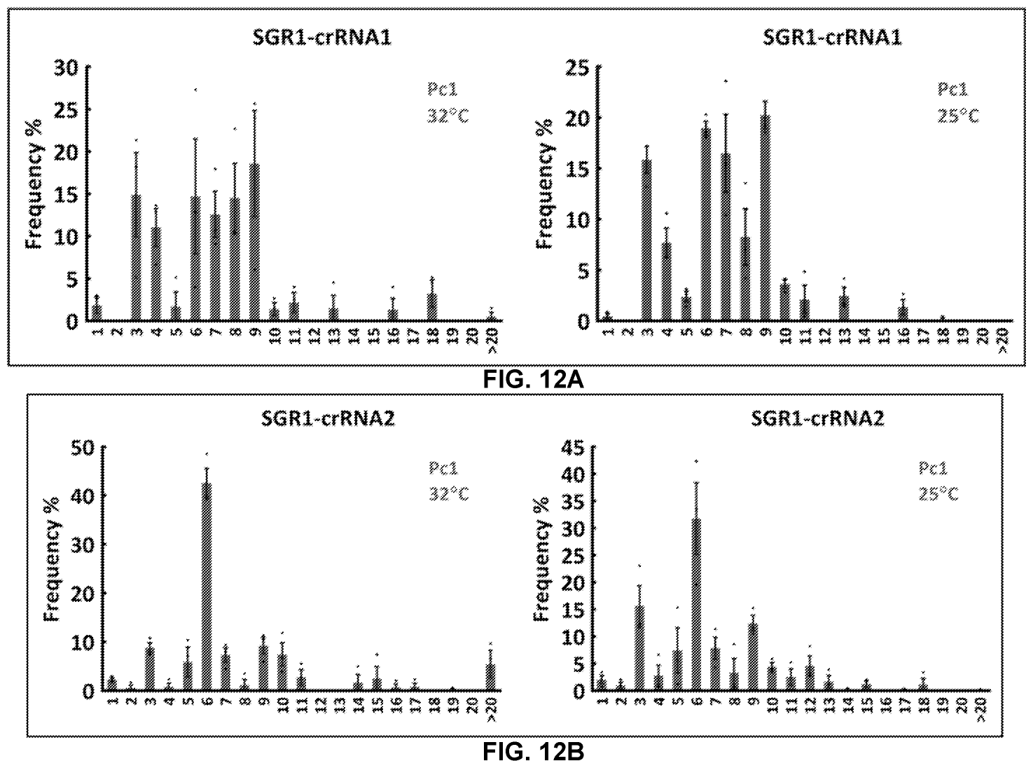

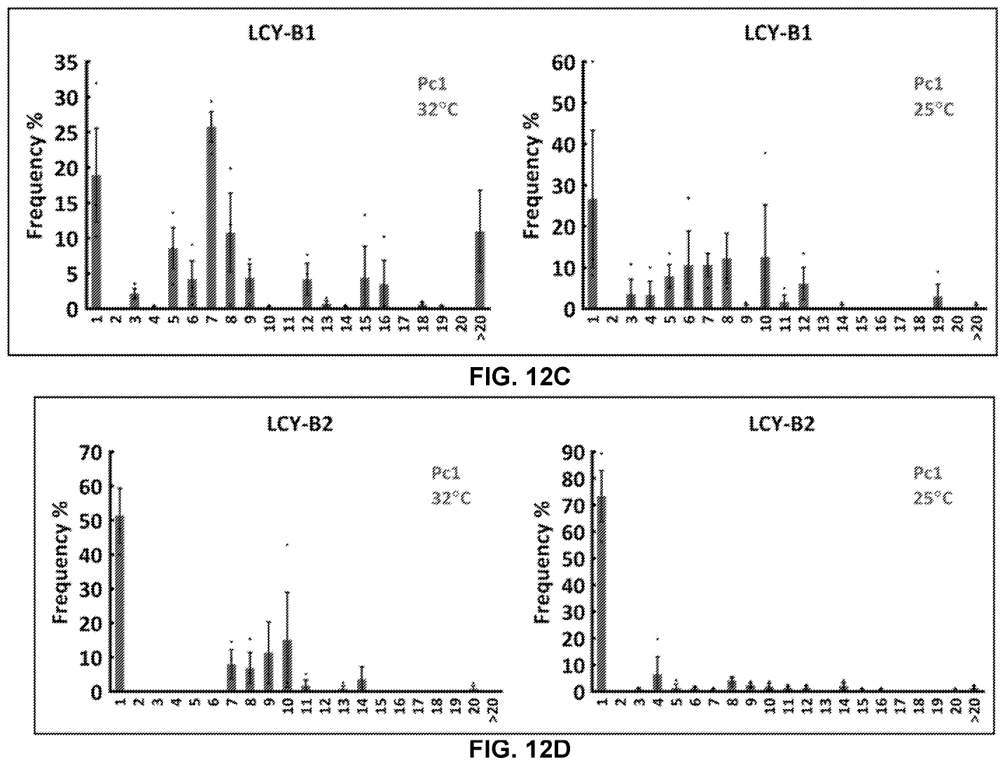

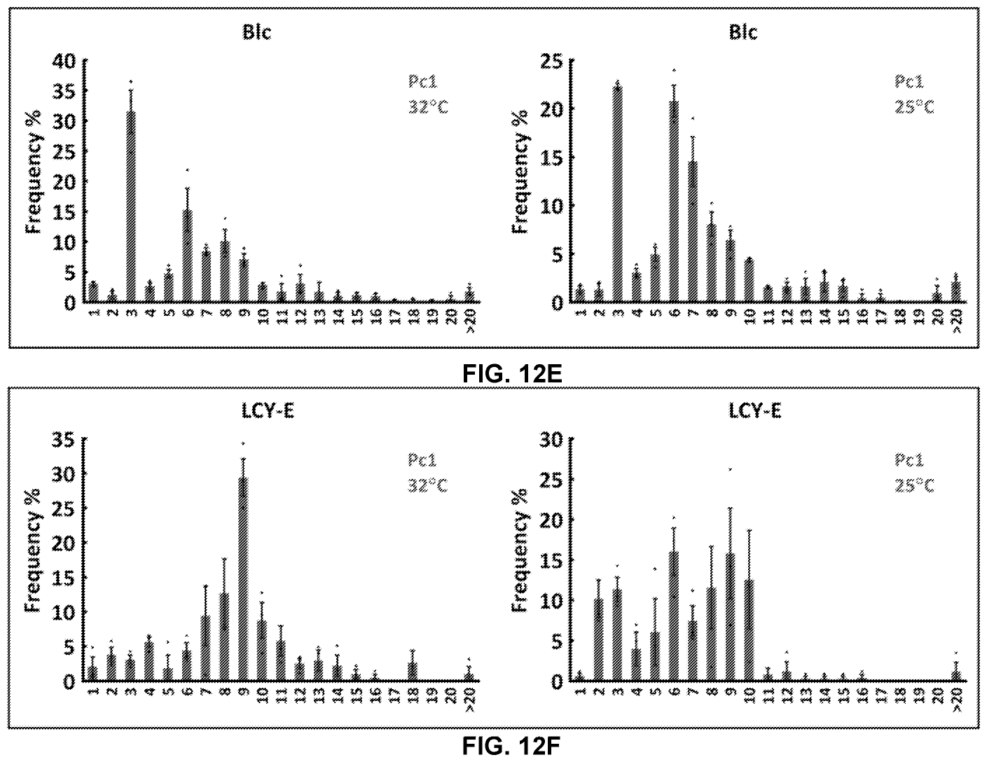

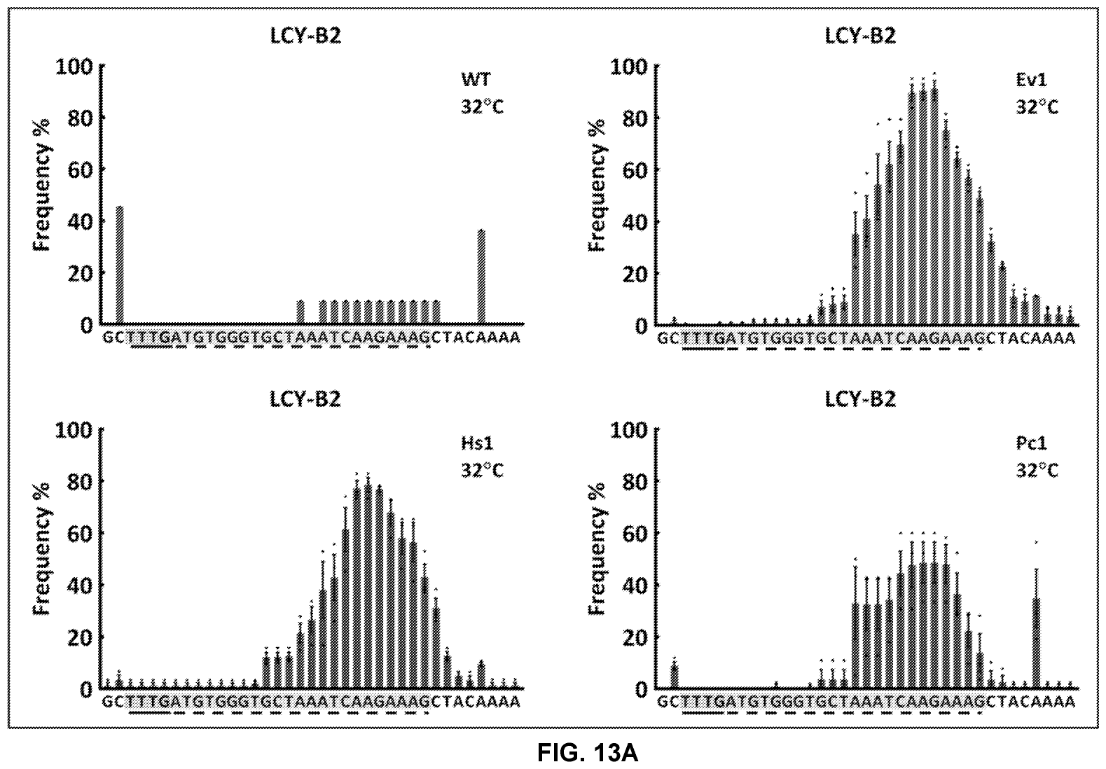

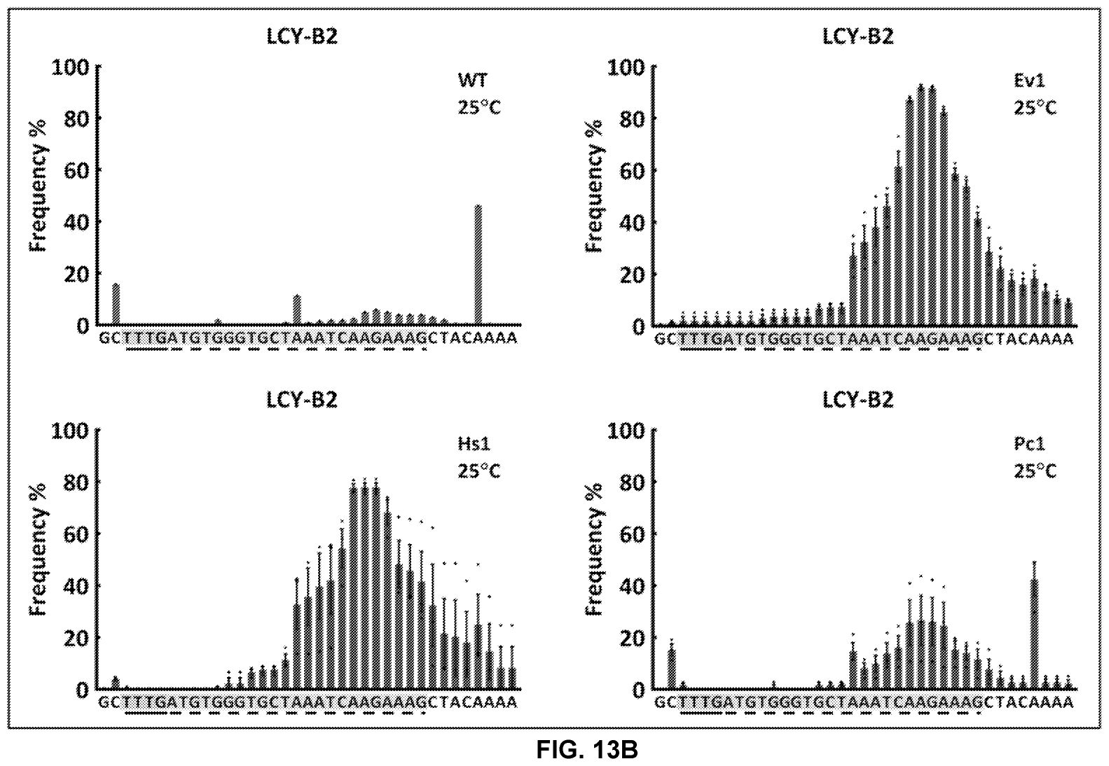

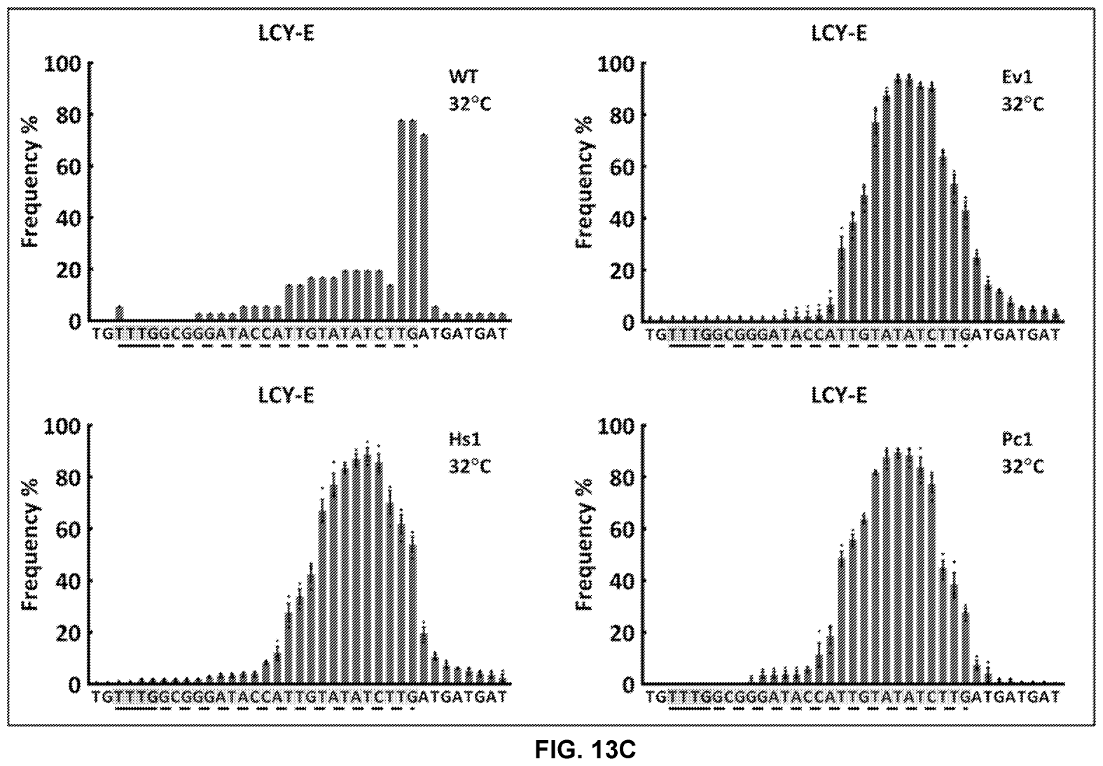

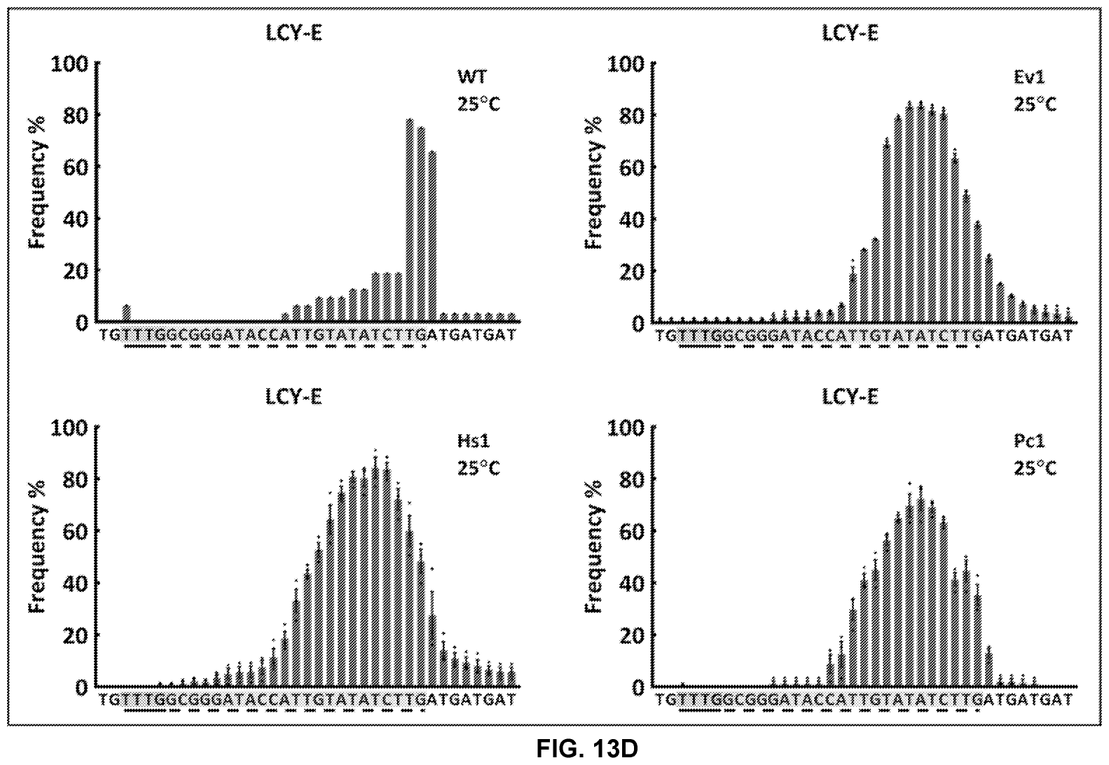

Amplicon sequencing using NGS revealed efficient genome editing by Ev1Cas12a and Hs1Cas12a at most target sites (FIG. 9A-F). At sites SGR1-crRNA2, Blc and LCY-E, Ex1Cas12a and HsCas12a showed comparable editing efficiencies to LbCas12a and Mb2Cas12a. However, Pc1Cas12a only showed efficient editing at the Ble site. Interestingly, slightly higher editing efficiencies were observed at 25° C. than 32° C. Similar to observations in rice, deletion is the predominant edit type in tomato protoplasts. Deletion sizes ranged from 3-13 bp and were not significantly affected by temperature (FIG. 10A-F, FIG. 11A-F, and FIG. 12A-F). Deletion position was about 12-23 bp away from the PAM and was also not significantly affected by temperature (FIG. 13A-D).

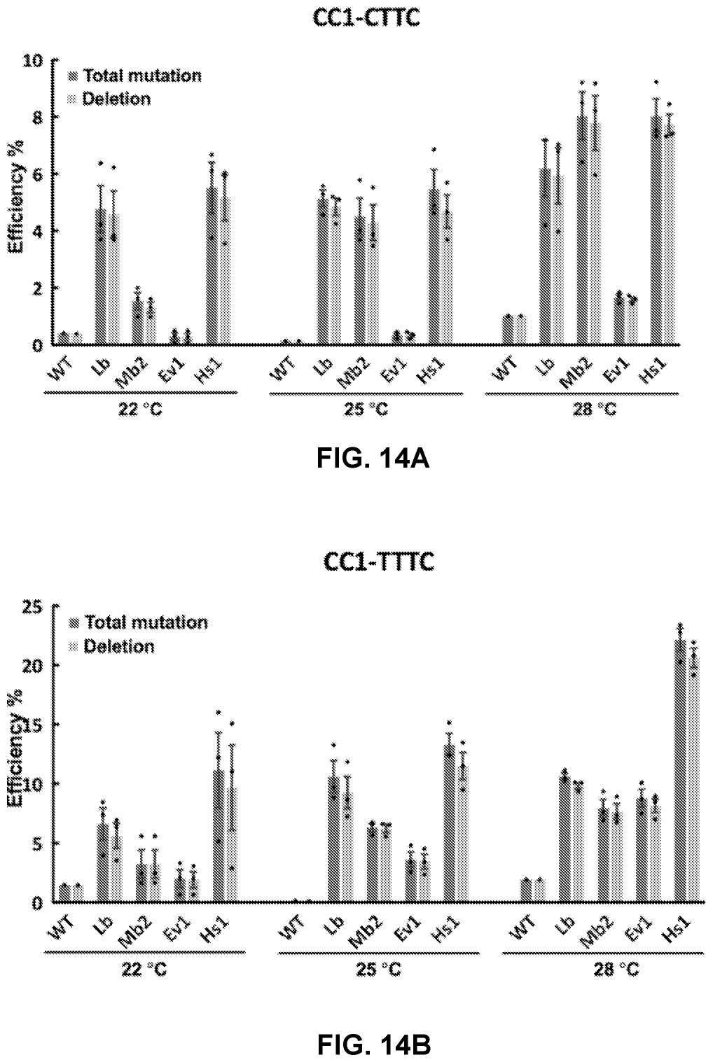

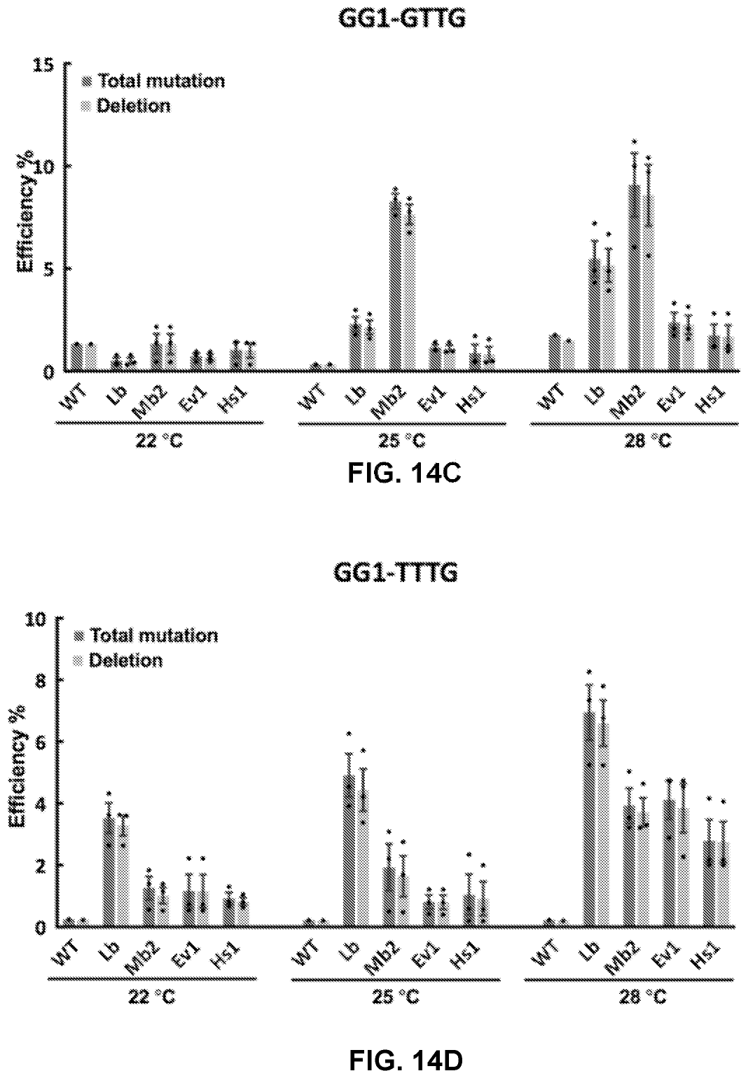

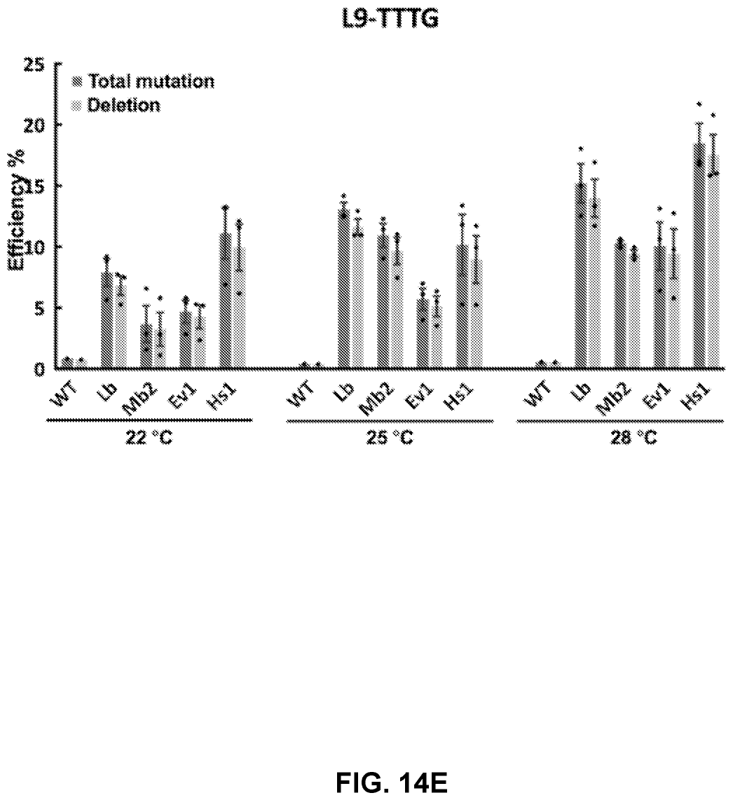

The data suggest that Ev1Cas12a and Hs1Cas12a can efficiently edit many target sites in the rice and tomato genomes. To further test the editing efficiency of both Cas12a orthologs at lower temperatures, multiplexed genome editing was performed with Ev1Cas12a and Hs1Cas12a in both rice and tomato protoplasts at 22° C. in addition to 25° C. and 32° C. In rice protoplasts, three crRNAs were used to target five sites L9-TTTG, CC1-CTTC, CC1-TTTC, GG1-GTTG and GG1-TTTG. At target sites L9-TTTG, CC1-CTTC, CC1-TTTC, Hs1Cas12a showed higher editing efficiency than LbCas12a and Mb2Cas12a regardless of temperature conditions (FIG. 14A-E). EvlCas12a also showed manifest editing efficiency especially at 28° C. For the low-activity sites GG1-GTTG and GG1-TTTG, both Ev1Cas12a and HsCas12a showed comparable editing efficiency to LbCas12a or Mb2Cas12a in some temperature conditions. In total, Hs1Cas12a showed comparable and even higher editing efficiency than LbCas12a and Mb2Cas12a in rice protoplast.

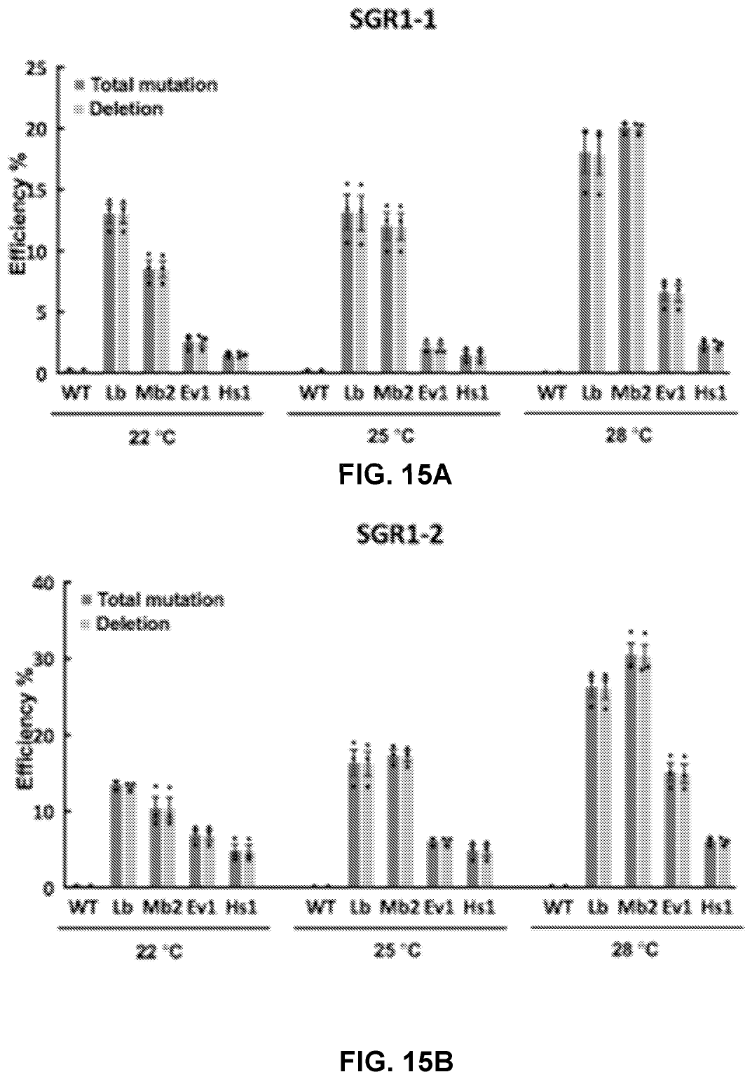

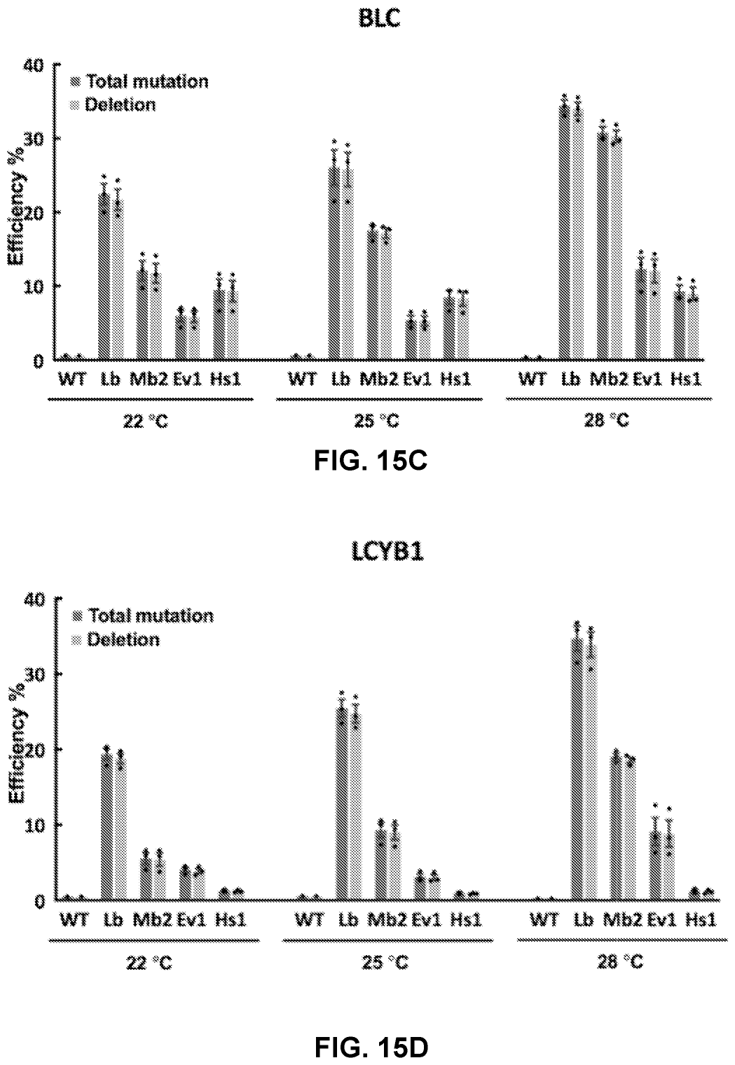

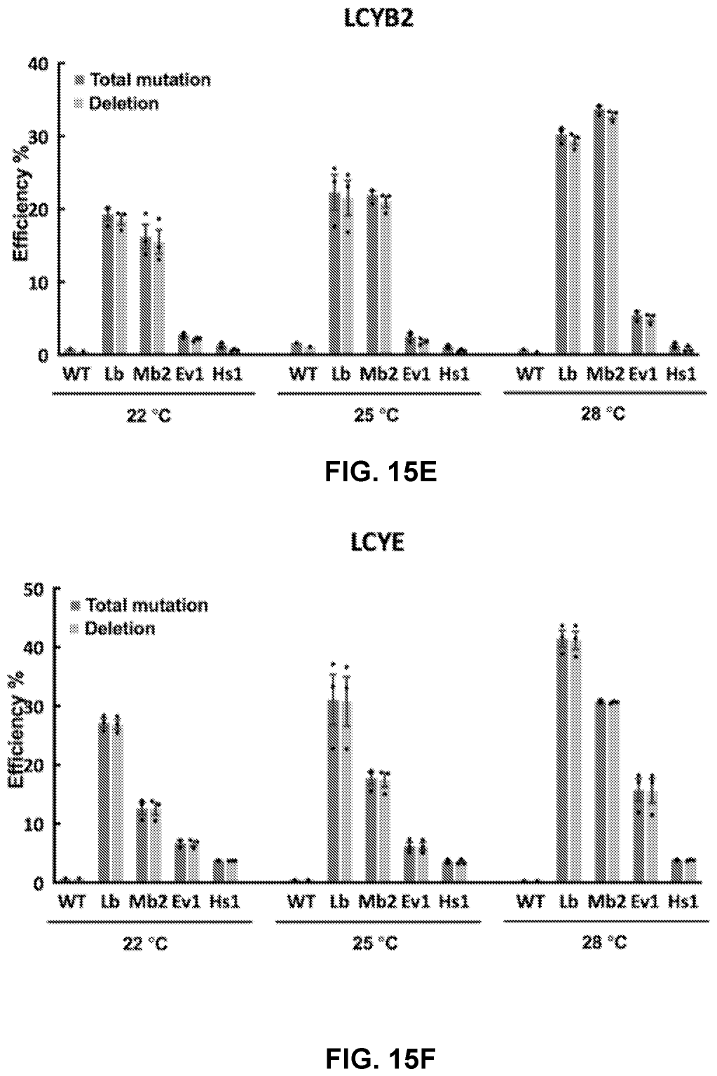

In tomato protoplasts, Hs1Cas12a showed manifest genome editing at four target sites (SGR1-1, SGR1-2, BLC, and LCYE) regardless of temperature conditions (FIG. 15A-F). Similarly, manifest genome editing of Ev1Cas12a was observed at all six target sites with highest editing efficiency at 28° C. Taken together, Hs1 is less sensitive to low temperature in both rice and tomato protoplast while Ev1Cas12a performed higher editing efficiency at 28° C.

In rice protoplast, four temperatures, 22° C., 25° C., 28° C., and 32° C., were compared. Like LbCas12a and Mb2Cas12a, Hs1Cas12a performed higher editing efficiency at 32° C. and 28° C. while Hs1Cas12a showed higher editing efficiency at CC1-TTTC and L9-TTTG sites under lower temperature. Previously, temperature sensitivity was reported to affect editing activity of Cas12a and editing activity was barely observed at 22° C. in both Arabidopsis and maize. However, Hs1Cas12a showed higher editing efficiency than LbCas12a at CC1-TTTC and L9-TTTC sites at 22° C., which indicates Hs1Cas12a may achieve high editing efficiency in plants such as Arabidopsis and maize. Interestingly, both Hs1Cas12a and Ev1Cas12a showed highest editing efficiency at 28° C. at all six target sites. In contrast to rice protoplast, higher editing efficiency was observed for Ev1Cas12a than for Hs1Cas12a in tomato protoplasts. These data suggest different Cas12a orthologs may have different genome editing activities in different plant species.

### Example 5: Genome Editing of Hs1Cas12a in Transgenic Rice Plants

Since Hs1Cas12a showed significant editing efficiency in rice protoplasts, its editing efficiency was further tested in rice transgenic To plants. Three crRNAs were multiplexed to target five sites L9-TTTG, CC1-TTTC, CC1-CTTC, GG1-TTTG and GG1-TTTG. Agrobacterium-mediated transformation was performed as previously described (Zhang et al. (2021) Nat Commun.12 (1): 1944). Regenerated shoots were cultured at 29° C. in 16h/8h (light/dark) condition. Young leaves of TO plants were used for DNA extraction using CTAB methods. Sixteen To plants were tested. The total editing efficiency and biallelic editing efficiency was 87.5%-100% and 50%-87.5%, respectively, at three target sites L9-TTTG, CC1-TTTC and CC1-CTTC. Table 2 shows editing efficiency of Hs1-Cas12a at five target sites in rice To plants. In the biallelic edited To plants, the deletion size was 5-11 bp, 8-9 bp and 8-9 bp at L9-TTTG, CC1-TTTC and CC1-CTTC, respectively, which is consistent with deletion size observed in rice protoplast. Table 3 shows the genotypes of biallelic mutant lines at the three target sites. Therefore, Hs1Cas12a can be used for highly efficient genome editing in rice to accelerate crop breeding and trait improvement.

TABLE 2

| | Tested T₀ | Edit | Biallelic edit |
|---|---|---|---|
| Target genes | lines | (number; ratio) | (number; ratio) |
| L9-TTTG | 16 | 16; 100% | 8; 50.0% |
| CC1-TTTC | 16 | 16; 100% | 14; 87.5% |
| GG1-TTTG | 16 | 0; 0.0% | 0; 0.0% |
| CC1-CTTC | 16 | 14; 87.5% | 10; 62.5% |
| GG1-GTTG | 16 | 0; 0.0% | 0; 0.0% |

TABLE 3

| Target | T₀ | |
|---|---|---|
| gene | Lines | T₀ mutation |
| L9- | WT | TGTTTGAAGAAGGGTTATGGCCAATGCT |
| TTTG | | TGGCCC (SEQ ID NO: 54) |
| | #9 | TGTTTGAAGAAGGGTTATG--------- |
| | | -GCCCC (SEQ ID NO: 55) |
| | | TGTTTGAAGAAGGGTTATG------GCT |
| | | TGGCCC (SEQ ID NO: 56) |
| | #12 | TGTTTGAAGAAGGGTTA----------- |
| | | -GCCCC (SEQ ID NO: 57) |
| | | TGTTTGAAGAAGGGTTATGGC------ |
| | | -TGCCCC (SEQ ID NO: 58) |
| CC1- | WT | GCTCTTTCTCCTGAGGAGCAAGAGCCA |
| TTTC | | TCACAGGTA (SEQ ID NO: 59) |
| | #3 | GCTCTTTCTCCTGAGGAGCA------- |
| | | --ACAGGTA (SEQ ID NO: 60) |
| | | GCTCTTTCTCCTGAGGAGCAAG----- |
| | | ---CAGGTA (SEQ ID NO: 61) |
| | #8 | GCTCTTTCTCCTGAGGAGCA------- |
| | | --ACAGGTA (SEQ ID NO: 60) |
| | | GCTCTTTCTCCTGAGGAGCAAG----- |
| | | ---CAGGTA (SEQ ID NO: 61) |
| CC1- | WT | TCCTTCTCCTGAGGAGCAAGAGCCATC |
| CTTC | | ACAGGTA (SEQ ID NO: 62) |
| | #5 | TCCTTCTCCTGAGGAGCAAG------- |
| | | -CAGGTA (SEQ ID NO: 63) |
| | | TCCTTCTCCTGAGGAGCA--------- |
| | | ACAGGTA (SEQ ID NO: 64) |
| | #6 | TCCTTCTCCTGAGGAGCAAG------- |
| | | -CAGGTA (SEQ ID NO: 63) |
| | | TCCTTCTCCTGAGGAGCA--------- |
| | | ACAGGTA (SEQ ID NO: 64) |

Sequences in italics indicate crRNA target sites and bold sequences indicate PAM sequence

### Example 6: Genome Editing of Hs1Cas12a and Ev1Cas12a in Poplar to Plants

The genome editing capability of HslCas12a and Ev1Cas12a were further tested in another dicot species, poplar, a woody plant model. The same Cas12a entry clones for rice genome editing were used. An entry clone of multiplexed six crRNAs driven by AtUBQ10 promoter was generated to target six sites in three genes (4CL1, PII and SVP) using a tandem ribozyme multiplexing system. Cas12a entry clones and the multiplexed crRNA clone were assembled into destination vector pYPQ202 by LR reaction to generate the final T-DNA vectors for poplar stable transformation.

Agrobacterium-mediated transformation was performed in poplar as described (Leple et al. (1992) Plant Cell Rep. 11 (3): 137-41). Regenerated shoots from the selection medium containing hygromycin were transferred to the rooting medium. Rooted plants were propagated and cultured at 25° C. in 16h/8h (light/dark) conditions. Young leaves were mixed with dilution buffer from Phire Plant Direct PCR Kit (ThermoFisher) to be further used for genotyping.

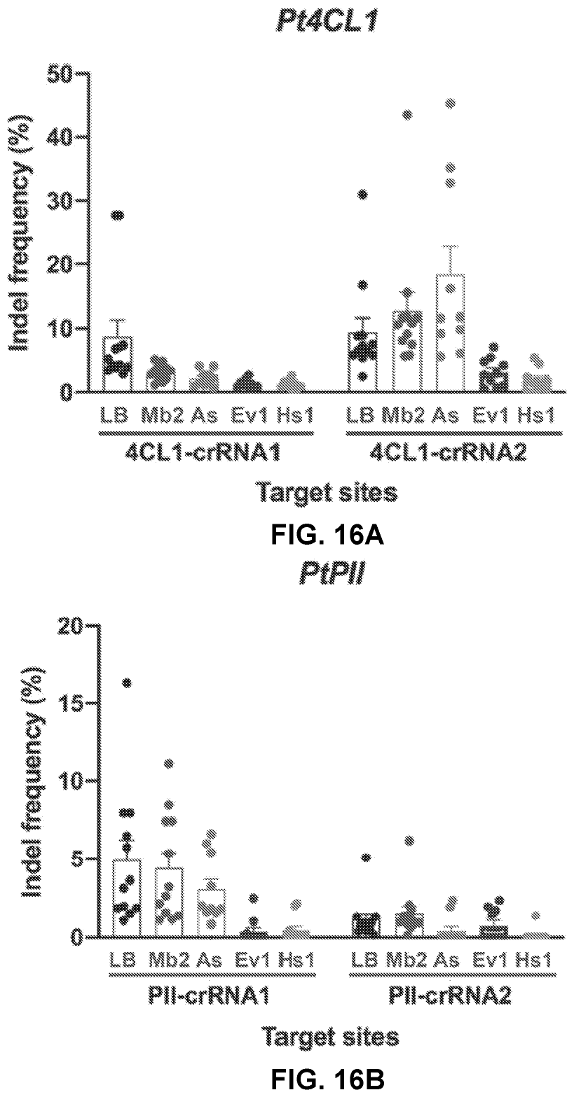

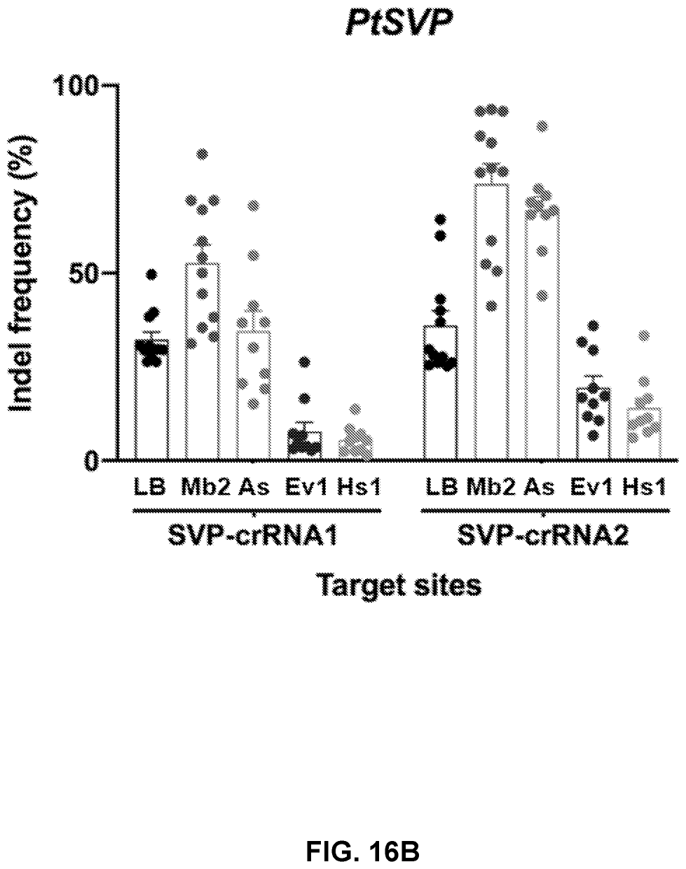

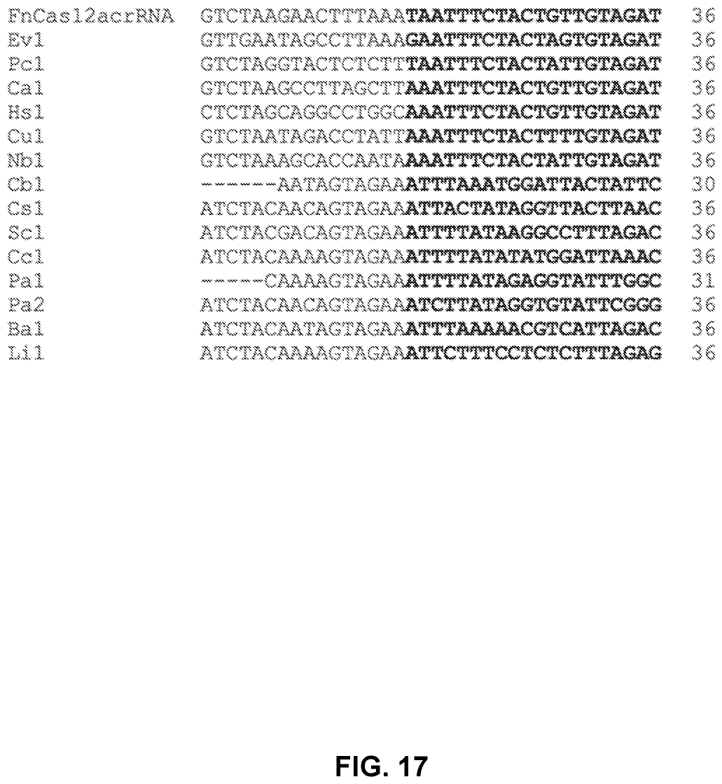

Six crRNAs were multiplexed to target three genes 4CL1, PII and SVP in poplar. Ten To plants were tested for each transformation. Ev1Cas12a and Hs1Cas12a showed up to 19.5% and 14.0% indel frequencies at two SVP target sites, respectively. Both Ev1Cas12a and Hs1Cas12a showed low indel frequencies at the other four target sites (4CL1-1, 4CL1-2, PII-1 and PII-2), for which low indel frequencies were also observed for LbCas12a, Mb2Cas12a and AsCas12a. The total editing efficiency of Ev1Cas12a and Hs1Cas12a at all six target sites is 20%-100% and 10%-100%, respectively (FIG. 16A-C). Table 4 shows the editing efficiency of Ps1Cas12a and Ev1Cas12a at the six target sites. No biallelic editing was detected. Thus, both Hs1Cas12a and Ev1Cas12a enable successful genome editing in poplar transgenic lines.

TABLE 4

| | |
|---|---|
| Ev1Cas12a | Hs1Cas12a |

| | Tested | Edit | Tested | Edit |
|---|---|---|---|---|
| Target | T₀ | (number; | T₀ | (number; |
| genes | lines | ratio)) | lines | ratio)) |
| 4CL1-1 | 10 | 6; 60% | 10 | 6; 60% |
| 4CL1-2 | 10 | 8; 80% | 10 | 6; 60% |
| PII-1 | 10 | 2; 20% | 10 | 2; 20% |
| PII-2 | 10 | 4; 40% | 10 | 1; 10% |
| SVP-1 | 10 | 10; 100% | 10 | 10; 100% |
| SVP-2 | 10 | 10; 100% | 10 | 10; 100% |

1. A method of modifying a nucleotide sequence at a target site in the genome of a plant cell, the method comprising: introducing into the plant cell
(i) a Cas12a polypeptide, or a polynucleotide encoding the Cas12a polypeptide, wherein the Cas12a polypeptide has at least 80%, at least 90%, at least 95%, at least 98%, or at least 99% sequence identity to the amino acid sequence set forth in SEQ ID NO: 23, 24, or 26; and
(ii) a DNA-targeting RNA, or a DNA polynucleotide encoding a DNA-targeting RNA, wherein the DNA-targeting RNA is capable of forming a complex with the Cas12a polypeptide and directing the complex to the target site.

2. The method of claim 1, wherein the polynucleotide encoding the Cas12a polypeptide has at least 80%, at least 90%, at least 95%, at least 98%, or at least 99% sequence identity to the nucleotide sequence set forth in SEQ ID NO: 6, 7, or 9.

3. The method of claim 1, wherein the Cas12a polypeptide comprises the amino acid sequence set forth in SEQ ID NO: 23, 24, or 26, or wherein the polynucleotide encoding the Cas12a polypeptide comprises the nucleotide sequence set forth in SEQ ID NO: 6, 7, or 9.

4. (canceled)

5. The method of claim 1, wherein the Cas12a polypeptide introduces a double-strand break (DSB) at the target site to produce the modified nucleotide sequence.

6. The method of claim 5, wherein the DSB at the target site comprises a staggered DSB.

7. The method of claim 1, wherein the modified nucleotide sequence comprises insertion of heterologous DNA into the genome of the plant cell, deletion of a nucleotide sequence from the genome of the plant cell, or mutation of at least one nucleotide in the genome of the plant cell.

8. The method of claim 1, wherein the plant cell is from a monocotyledonous or a dicotyledonous species.

9. The method of claim 1, wherein the plant cell is from Oryza sativa, Solanum lycopersicum, or Populus sp.

10. The method of claim 1, wherein expression of the Cas12a polypeptide is under the control of an inducible promoter, a constitutive promoter, a cell type-specific promoter, or a developmentally preferred promoter.

11. The method of claim 1, wherein the DNA-targeting RNA comprises: (a) a first segment comprising a nucleotide sequence that is complementary to a sequence in the target DNA; and (b) a second segment that interacts with the Cas12a polypeptide.

12. The method of claim 1, wherein the target site is located immediately 3′ of a PAM site in the genome of the plant cell, and wherein the PAM site comprises TTTV.

13. (canceled)

14. The method of claim 1, wherein the introducing is at a temperature from about 22° C. to about 32° C.

15. The method of claim 1, wherein the polynucleotide sequence encoding the Cas12a polypeptide is codon-optimized for expression in a plant cell.

16. The method of claim 1, wherein the Cas12a polypeptide comprises one or more mutations that reduce or eliminate the nuclease activity of the Cas12a polypeptide; or wherein the Cas12a polypeptide has nickase activity.

17. (canceled)

18. The method of claim 1, wherein the Cas12a polypeptide is fused to a deaminase domain, and wherein the modified nucleotide sequence comprises a base edit at the target site.

19. The method of claim 1, wherein the Cas12a polypeptide is fused to a reverse transcriptase domain, and wherein the DNA-targeting RNA is a prime editing guide RNA (pegRNA).

20. The method of claim 1, wherein polynucleotide encoding the Cas12a polypeptide is present in a vector.

21. The method of claim 1, further comprising regenerating a plant from the plant cell.

22-23. (canceled)

24. A method of modulating the expression of a target gene in a plant cell, the method comprising: introducing into the plant cell
(i) a Cas12a polypeptide, or a polynucleotide encoding the Cas12a polypeptide, wherein the Cas12a polypeptide has at least 80%, at least 90%, at least 95%, at least 98%, or at least 99% sequence identity to the amino acid sequence set forth in SEQ ID NO: 23, 24, or 26, and wherein the Cas12a polypeptide comprises one or more mutations that reduce or eliminate the nuclease activity of the Cas12a polypeptide; and
(ii) a DNA-targeting RNA, or a DNA polynucleotide encoding a DNA-targeting RNA, wherein the DNA-targeting RNA is capable of forming a complex with the Cas12a polypeptide and directing the complex to the target site.

25. The method of claim 24, wherein the target gene is upregulated or downregulated.

26. The method of claim 24, wherein the Cas12a polypeptide is fused to a transcriptional activation domain or a transcriptional repression domain.

27. A nucleic acid construct comprising a polynucleotide encoding a Cas12a polypeptide, wherein the Cas12a polypeptide has at least 80%, at least 90%, at least 95%, at least 98%, or at least 99% sequence identity to the amino acid sequence set forth in SEQ ID NO: 23, 24, or 26, wherein the polynucleotide is operably linked to a heterologous promoter that is operable in a plant cell.

28. The nucleic acid construct of claim 27, wherein the polynucleotide encoding the Cas12a polypeptide has at least 80%, at least 90%, at least 95%, at least 98%, or at least 99% sequence identity to the nucleotide sequence set forth in SEQ ID NO: 6, 7, or 9.

29. The nucleic acid construct of claim 27, wherein the Cas12a polypeptide comprises the amino acid sequence set forth in SEQ ID NO: 23, 24, or 26, or wherein the polynucleotide encoding the Cas12a polypeptide comprises the nucleotide sequence set forth in SEQ ID NO: 6, 7, or 9.

30. (canceled)

31. The nucleic acid construct of claim 27, wherein the heterologous promoter is an inducible promoter, a constitutive promoter, a cell type-specific promoter, or a developmentally preferred promoter.

32. The nucleic acid construct of claim 27, wherein the polynucleotide encoding the Cas12a polypeptide is codon-optimized for expression in a plant cell.

33. The nucleic acid construct of claim 27, wherein the Cas12a polypeptide has reduced or eliminated nuclease activity; or wherein the Cas12a polypeptide has nickase activity.

34. (canceled)

35. The nucleic acid construct of claim 27, wherein the polynucleotide encodes a Cas12a polypeptide fused to a transcriptional activation domain, a transcriptional repression domain, a deaminase domain, or a reverse transcriptase domain.

36. (canceled)

37. A plant or a plant cell comprising:
(i) a Cas12a polypeptide, or a polynucleotide encoding the Cas12a polypeptide, wherein the Cas12a polypeptide has at least 80%, at least 90%, at least 95%, at least 98%, or at least 99% sequence identity to the amino acid sequence set forth in SEQ ID NO: 23, 24, or 26; and
(ii) a DNA-targeting RNA, or a DNA polynucleotide encoding a DNA-targeting RNA, wherein the DNA-targeting RNA is capable of forming a complex with the Cas12a polypeptide and directing the complex to a target site.

38. The plant or plant cell of claim 37, wherein the polynucleotide encoding the Cas12a polypeptide has at least 80%, at least 90%, at least 95%, at least 98%, or at least 99% sequence identity to the nucleotide sequence set forth in SEQ ID NO: 6, 7, or 9.

39. The plant or plant cell of claim 37, wherein the Cas12a polypeptide comprises the amino acid sequence set forth in SEQ ID NO: 23, 24, or 26; or
wherein the polynucleotide encoding the Cas12a polypeptide comprises the nucleotide sequence set forth in SEQ ID NO: 6, 7, or 9.

40. (canceled)

41. The plant or plant cell of claim 37, wherein the plant is a monocotyledonous or a dicotyledonous plant.

42. The plant or plant cell of claim 37, wherein the plant is Oryza sativa, Solanum lycopersicum, or Populus sp.

43. The plant or plant cell of claim 37, wherein expression of the Cas12a polypeptide is under the control of an inducible promoter, a constitutive promoter, a cell type-specific promoter, or a developmentally preferred promoter.

44. The plant or plant cell of claim 37, wherein the polynucleotide sequence encoding the Cas12a polypeptide is codon-optimized for expression in a plant cell.
